SEQUENCE LISTING (26 sequences)

SEQ ID NO: 1 - DNA, 74 bp, unidentified
  source 1..74: mol_type = unassigned DNA
aagcggcgctgcagggcggccagctgtagctggagtagatcgtacgcggctttcggaccttcttgggcttccca

SEQ ID NO: 2 - DNA, 75 bp, unidentified
  source 1..75: mol_type = unassigned DNA
aagcggcgctgcagggcggccagctggtagctggagtagatcgtacgcggctttcggaccttcttgggcttccca

SEQ ID NO: 3 - DNA, 66 bp, unidentified
  source 1..66: mol_type = unassigned DNA
ggccttctggaagcggcgctgcagggcggccagctgtagctggagtagatcgtacgcggctttcgg

SEQ ID NO: 4 - DNA, 66 bp, unidentified
  source 1..66: mol_type = unassigned DNA
ggccttctggaagcggcgctgcagggcggccagctggtagctggagtagatcgtacgcggctttcg

SEQ ID NO: 5 - DNA, 74 bp, unidentified; identical to SEQ ID NO: 1
  source 1..74: mol_type = unassigned DNA

SEQ ID NO: 6 - DNA, 75 bp, unidentified; identical to SEQ ID NO: 2
  source 1..75: mol_type = unassigned DNA

SEQ ID NO: 7 - protein, 71 aa, synthetic construct
MGDRRKEMDKVYRTAYKRITSTPDKEKRKEVVKEATEQLRRIAKDEEEKKKAAYMISFLKTLGLEHHHHHH

SEQ ID NO: 8 - protein, 21 aa, synthetic construct
MSHHHHHHHHSENLYFQSGGG

SEQ ID NO: 9 - DNA, 19 bp, synthetic construct
  source 1..19: mol_type = other DNA
tccctggagaagagctacg

SEQ ID NO: 10 - DNA, 20 bp, synthetic construct
  source 1..20: mol_type = other DNA
gtagtttcgtggatgccaca

SEQ ID NO: 11 - DNA, 20 bp, synthetic construct
  source 1..20: mol_type = other DNA
ctggacactaaacccctgcc

SEQ ID NO: 12 - DNA, 21 bp, synthetic construct
  source 1..21: mol_type = other DNA
catgtggactgggctgcagac

SEQ ID NO: 13 - DNA, 20 bp, synthetic construct
  source 1..20: mol_type = other DNA
tgtttccctttcctgtgtgg

SEQ ID NO: 14 - DNA, 20 bp, synthetic construct
  source 1..20: mol_type = other DNA
ttatatcgccttgggcattg

SEQ ID NO: 15 - DNA, 18 bp, synthetic construct
  source 1..18: mol_type = other DNA
ctcgtcaaagccgagagc

SEQ ID NO: 16 - DNA, 19 bp, synthetic construct
  source 1..19: mol_type = other DNA
cggttcgtcttgtgtttgc

SEQ ID NO: 17 - DNA, 23 bp, synthetic construct
  source 1..23: mol_type = other DNA
gagcaacacggatggatgatttc

SEQ ID NO: 18 - DNA, 23 bp, synthetic construct
  source 1..23: mol_type = other DNA
cactcttgtcacgcacagcctta

SEQ ID NO: 19 - DNA, 21 bp, synthetic construct
  source 1..21: mol_type = other DNA
ttgaactcgttgacgctgagg

SEQ ID NO: 20 - DNA, 21 bp, synthetic construct
  source 1..21: mol_type = other DNA
gcagaaccactgacagagcta

SEQ ID NO: 21 - DNA, 20 bp, synthetic construct
  source 1..20: mol_type = other DNA
gaaacagccatagagggcaa

SEQ ID NO: 22 - DNA, 23 bp, synthetic construct
  source 1..23: mol_type = other DNA
tggttttccagagtcttcagtga

SEQ ID NO: 23 - DNA, 20 bp, synthetic construct
  source 1..20: mol_type = other DNA
catggcgggtaacgatgaaa

SEQ ID NO: 24 - DNA, 22 bp, synthetic construct
  source 1..22: mol_type = other DNA
gtgaagacggttatggtcaagg

SEQ ID NO: 25 - DNA, 21 bp, synthetic construct
  source 1..21: mol_type = other DNA
gacagacaagaaggaggaaac

SEQ ID NO: 26 - DNA, 19 bp, synthetic construct
  source 1..19: mol_type = other DNA
gctctcactggtggtatct

(19) United States
(12) Patent Application Publication
(10) Pub. No.: US 2026/0117184 A1
(43) Pub. Date: Apr. 30, 2026

(54) SYSTEM AND METHOD TO DIRECT THE DIFFERENTIATION OF PLURIPOTENT STEM CELL-DERIVED ODONTOBLASTS

(71) Applicant: UNIVERSITY OF WASHINGTON, Seattle, WA (US)

(72) Inventors: Anjali PATNI, Seattle, WA (US); David BAKER, Seattle, WA (US); Hannele RUOHOLA-BAKER, Seattle, WA (US); Sesha HANSON-DRURY, Seattle, WA (US); Ammar ALGHADEER, Seattle, WA (US); Julie MATHIEU, Seattle, WA (US)

(73) Assignee: UNIVERSITY OF WASHINGTON, Seattle, WA (US)

(21) Appl. No.: 19/104,519

(22) PCT Filed: Aug. 15, 2023
(86) PCT No.: PCT/US2023/072209

§ 371 Date: Feb. 18, 2025

Related U.S. Application Data
(60) Provisional application No. 63/398,510, filed on Aug. 16, 2022.
(60) Provisional application No. 63/471,426, filed on Jun. 6, 2023.

(51) Int. Cl. C12N 5/077; A61K 35/32

(52) U.S. Cl. CPC C12N 5/0654; A61K 35/32; C12N 2501/115; C12N 2501/119; C12N 2501/15; C12N 2501/155; C12N 2501/41; C12N 2501/415; C12N 2506/45

(57) ABSTRACT

The technology as described herein relates to discovery of methods for generating odontoblasts or organoids thereof in vitro and compositions that utilize the generated odontoblasts for restoring tooth structure.

### CROSS REFERENCE TO RELATED APPLICATIONS

This application is a 371 National Phase Entry of International Patent Application No. PCT/US2023/072209 filed Aug. 15, 2023 which claims benefit under 35 U.S.C. § 119(e) of U.S. Provisional Application No. 63/398,510 filed Aug. 16, 2022 and U.S. Provisional Application No. 63/471,426 filed Jun. 6, 2023, the contents of all of which are incorporated herein by reference in their entireties.

### GOVERNMENT SUPPORT

This invention was made with government support under Grant Nos. 1P01GM081619 and R01GM083867 and R01GM097372 and R01GM97372-03S1 and T90DE021984, awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Aug. 8, 2023, is named “034186-000104WOPT_SL.xml” and is 24,292 bytes in size.

### TECHNICAL FIELD

The technology described herein relates to the in vitro generation of cells useful in dental repair.

### BACKGROUND

There is much interest in the dental field for methods of maintaining or repairing damage to tooth structure and enamel. Replacing lost tooth structure to restore dental form and function has been a clinical goal for over 13,000 years, with one of the earliest examples of dental treatment discovered in a hunter-gatherer of the Late Upper Paleolithic era with a dental filling composed of organic material. The desire to restore the form and function of missing tooth structure remains a prime concern of both clinicians and the general public today. In a recent Reddit thread polling what missing capability of the human body was most desired by community members, tooth regeneration was one of the most upvoted responses. This comes as no surprise to oral health clinicians and scientists alike, as the number of dental patients treated for missing tooth structure is substantial and restorative treatment limitations pose a significant clinical problem. Tooth structure is commonly lost due to dental caries, trauma, periodontal disease, and congenital defects. The Global Burden of Diseases Study 2017 (GBD 2017) shows that, regardless of gender, oral disorders had the greatest age-standardized prevalence and incidence in the world, persisting in this ranking since 1990. Untreated dental caries is the most prevalent disease globally, with the Center for Disease Control finding that 90% of adults in the United States suffer from dental caries. Further, dental pulp disease was the primary diagnosis for over 400,000 emergency department visits in the U.S. in 2006, highlighting the need for significant resources to restore both the dental pulp and the mineralized dentin tooth structure it produces. The current method to return form and function to the lost tooth structure (e.g. fillings, crowns) can initiate a continuous cycle of restoration replacement, each replacement leading to increased tooth structure loss due to preparation requirements, recurrent caries, or fracture. This process, known as the “tooth cycle of death”, can ultimately lead to tooth removal and replacement with a dental implant, currently one of the best tooth alternatives. Importantly, after 9 years 45% of dental implants develop peri-implantitis, an inflammatory process that can lead to loss of the implant and surrounding bone. At this stage the patient often suffers from insufficient bone levels to support a new dental implant, leaving both the patient and clinician in a treatment quandary. Regenerative dentistry seeks to produce stem cell tools to regenerate missing tooth structure. The need for a tooth organoid is paramount. The field of dentistry has a history of pioneering regenerative therapy, with the application of calcium hydroxide to induce reparative dentinogenesis in cases of pulp exposure since the 1800's. Since then, advances have been made in developing therapeutics that lead to more efficient reparative dentin formation. Mineral Trioxide Aggregate (MTA) leads to dentin bridge formation within the pulp. However, as MTA is not biodegradable, dentin formation is limited to within the pulp, preventing replacement of missing tooth structure from extending outward from the dental pulp. Biodegradable collagen sponges soaked in WNT agonists delivered to sites of pulpal injury showed increased mineralization compared to MTA in murine models. Importantly, mineralization extends outside the dental pulp into the access site created by the dental bur. While this finding holds much promise for regenerating lost dentin in vital teeth with a live dental pulp, these methods of dentin regeneration are not applicable to patients with necrotic pulpal tissue. These patients have lost both their primary odontoblasts capable of secreting tertiary dentin and stem cells capable of differentiating to secondary odontoblasts, and thus require a new cell source for pulpal regeneration.

### SUMMARY

The methods and compositions described herein are based, in part, on the discovery of methods for generating odontoblasts or organoids thereof in vitro from induced pluripotent stem cells (iPSCs). Also provided herein are compositions comprising in vitro-derived odontoblasts for administration or transplantation into a subject to induce dentin production, e.g., to treat structural tooth defects, dentin loss or demineralization. Also provided herein are mineralized products produced using the odontoblast or odontoblast organoids produced as described herein.

Accordingly in one aspect described herein is a method of preparing an odontoblast, the method comprising, in order a) contacting at day zero, in culture, a pluripotent stem cell with a TGF-β/SMAD inhibitor; b) adding a WNT activator to the culture in (a) at day 2; c) enriching the culture of step (b) for a population of induced neural crest stem cells; and d) contacting the population of induced neural crest stem cells enriched in (c) with an odontoblast differentiation medium comprising a BMP pathway agonist, an FGF pathway agonist, and a Hedgehog pathway agonist, and incubating to generate a population of odontoblast cells expressing dentin sialophosphoprotein (DSPP).

In one embodiment of this or other aspects described herein, the pluripotent stem cell is an induced pluripotent stem cell.

In another embodiment of this or other aspects described herein, steps (a) through (c) are performed in a basal neural maintenance medium (BNMM) to which the SMAD inhibitor and WNT activator are successively added or added and removed.

In another embodiment of this or other aspects described herein, the BNMM comprises: Dulbecco's Modified Eagle Medium F12+glutamine: neurobasal medium (1:1), wherein the neurobasal medium comprises a N2 supplement, B27, Glutamax, ITS-A, b-mercaptoethanol, and non-essential amino acids (NEAA).

In another embodiment of this or other aspects described herein, the SMAD inhibitor comprises SB431542 and LDN193189.

In another embodiment of this or other aspects described herein, the SB431542 is removed at day 4 of culture, and LDN193189 is removed at day 3 of culture.

In another embodiment of this or other aspects described herein, the WNT activator is added from day 2 to day 11 of the method.

In another embodiment of this or other aspects described herein, the WNT activator is a GSK-3 inhibitor.

In another embodiment of this or other aspects described herein, the GSK-3 inhibitor is CHIR99021.

In another embodiment of this or other aspects described herein, the CHIR99021 is added at 3 mM.

In another embodiment of this or other aspects described herein, the enriching step (c) comprises selection of cells expressing p75^{NTR}.

In another embodiment of this or other aspects described herein, the selection of cells expressing p75 comprises cell sorting with anti-p75 magnetic beads.

In another embodiment of this or other aspects described herein, the enriching step (c) is performed when a majority of the differentiating cells expresses p75^{NTR}.

In another embodiment of this or other aspects described herein, the enriching step (c) is performed at day 11.

In another embodiment of this or other aspects described herein, the BMP pathway agonist comprises BMP4.

In another embodiment of this or other aspects described herein, the FGF pathway agonist is selected from bFGF, FGF8b and an FGF receptor minibinder.

In another embodiment of this or other aspects described herein, the FGF receptor mini binder is selected from mb7 or mb6 receptor mini binder.

In another embodiment of this or other aspects described herein, the Hedgehog pathway agonist comprises Smoothened agonist (SAG).

In another embodiment of this or other aspects described herein, the odontoblast cells further express MSX1 and S100A13.

In another embodiment of this or other aspects described herein, the pluripotent stem cell is human.

In another embodiment of this or other aspects described herein, the pluripotent stem cell has a mutation inactivating expression or activity of DLX3.

In another embodiment of this or other aspects described herein, the pluripotent stem cells are seeded on tissue culture plates coated with an extracellular matrix composition.

In another embodiment of this or other aspects described herein, the extracellular matrix composition comprises a natural or a synthetic extracellular matrix composition.

In another embodiment of this or other aspects described herein, the pluripotent stem cells are grown to confluence prior to step (a).

In another embodiment of this or other aspects described herein, the iPSCs are cultured to confluence in mTeSR1 stem cell medium.

In another embodiment of this or other aspects described herein, confluent iPS cells are switched to a basal neural crest maintenance medium at day zero of differentiation.

In another embodiment of this or other aspects described herein, the TGF-β/SMAD inhibitors are added for at least 3 days.

In another embodiment of this or other aspects described herein, induced neural crest cell expresses p75, AP-2α, NESTIN, and/or PAX3.

In another embodiment of this or other aspects described herein, the selecting comprises selecting for a neural crest marker from the group consisting of p75, AP-2α, NESTIN, and PAX3.

In another embodiment of this or other aspects described herein, the odontoblast medium comprises Dulbecco's Modified Eagle Medium+Glutamax, dexamethasone, fetal bovine serum, b-glycerophosphate, and L-ascorbic acid.

In another embodiment of this or other aspects described herein, the BMP4 pathway agonist is added from Day 11 to Day 17 of differentiation.

In another embodiment of this or other aspects described herein, the BMP4 pathway agonist is at a concentration from 25 ng/mL to 100 ng/mL.

In another embodiment of this or other aspects described herein, the BMP4 pathway agonist is added from Day 17 to Day 26 of differentiation.

In another embodiment of this or other aspects described herein, the BMP4 pathway agonist is 50 ng/mL.

In another embodiment of this or other aspects described herein, SAG is added at 200 nM to 1 μM.

In another embodiment of this or other aspects described herein, SAG is added at 400 nM.

In another aspect, described herein is a human odontoblast produced by the method of any one of the embodiments described herein, wherein the FGF agonist is an FGF receptor mini binder, and the odontoblast exhibits at least 10% greater mineralization than an odontoblast differentiated without the FGF receptor mini binder.

In another aspect, described herein is a composition comprising an in vitro-differentiated human odontoblast and a biodegradable scaffold.

In one embodiment of this or other aspects described herein, the biodegradable scaffold comprises a PLGA polymer.

In another aspect, described herein is a tooth-repair composition comprising an in vitro-differentiated odontoblast.

In one embodiment of this or other aspects described herein, the composition further comprises a biodegradable scaffold.

In another embodiment of this or other aspects described herein, the biodegradable scaffold comprises PLGA polymer.

In another embodiment of this or other aspects described herein, the composition further comprising an in vitro differentiated ameloblast.

In another aspect, described herein is a co-culture comprising an in vitro-differentiated odontoblast and an in vitro-differentiated ameloblast.

In one embodiment of this or other aspects described herein, the co-culture comprises only in vitro-differentiated odontoblasts and in vitro-differentiated ameloblasts.

In another aspect, described herein is a co-culture comprising an odontoblast produced by the method of any one of the embodiments described herein and an in vitro-differentiated ameloblast.

In another aspect, described herein is a cultured organoid comprising an in vitro-differentiated odontoblast, wherein mineralization as measured by Alizarin red staining (ARS) is at least 10% greater than that occurring in culture that is not exposed to a receptor mini binder.

In one embodiment of this or other aspects described herein, the odontoblast is differentiated from an iPS cell.

In another embodiment of this or other aspects described herein, the odontoblast is human.

In another embodiment of this or other aspects described herein, the receptor mini binder is selected from mb7 or mb6 receptor mini binder.

In another aspect, described herein is a tooth comprising a dental repair composition comprising tertiary dentin produced by an in-vitro differentiated odontoblast.

In one embodiment of this or other aspects described herein, the dental repair composition further comprises calcium phosphate or hydroxyapatite.

In another embodiment of this or other aspects described herein, the dental repair composition further comprises one or more of amelogenin and enamelin.

In another aspect, described herein is a dental repair composition comprising tertiary dentin produced by an in vitro-differentiated cell.

In one embodiment of this or other aspects described herein, the composition further comprises enamel produced by an in vitro-differentiated cell.

In another embodiment of this or other aspects described herein, the composition further comprises hydroxyapatite or calcium phosphate.

In another embodiment of this or other aspects described herein, the composition further comprises one or more of amelogenin and enamelin.

In another embodiment of this or other aspects described herein, the in vitro-differentiated cell is an odontoblast differentiated from an iPS cell.

In another embodiment of this or other aspects described herein, the iPS cell is a human iPS cell.

In another aspect, described herein is a method of repairing a tooth, the method comprising contacting a tooth with a dental repair composition of any one of the embodiments described herein.

In another aspect, described herein is a method of treating a dental disease or disorder, the method comprising administering a composition comprising an in vitro-differentiated odontoblast to a subject in need thereof.

In one embodiment of this or other aspects described herein, a dental disease or disorder is selected from Tricho-Dento-Osseous (TDO) syndrome, amelogenesis imperfecta, periodontal disease, periodontitis, caries, pericoronitis, pulpitis, enamel hypoplasia, defects of dentition, and tartar.

In another embodiment of this or other aspects described herein, the odontoblast is differentiated from an iPS cell.

In another embodiment of this or other aspects described herein, the odontoblast is a human odontoblast.

In another embodiment of this or other aspects described herein, the iPS cell is derived from the subject.

In another aspect, described herein is a method of preparing a tooth structure repair composition, the method comprising culturing an in vitro-differentiated odontoblast.

In one embodiment of this or other aspects described herein, the odontoblast is differentiated from an iPS cell.

In another embodiment of this or other aspects described herein, the odontoblast is human.

In another embodiment of this or other aspects described herein, the odontoblast is in an organoid.

In another aspect, described herein is a method of screening for an agent that modulates dentin and/or enamel production, the method comprising contacting an in vitro-differentiated odontoblast or an organoid comprising an in vitro differentiated odontoblast with a candidate agent, and detecting a change in tertiary dentin expression.

In one embodiment of this or other aspects described herein, the odontoblast is differentiated from an iPS cell.

In another embodiment of this or other aspects described herein, the odontoblast is differentiated from an iPS cell by the method of any one of the embodiments described herein.

In another embodiment of this or other aspects described herein, the iPS cell is derived from an individual with a defect in enamel production.

In another embodiment of this or other aspects described herein, the defect in enamel production comprises Tricho-Dento-Osseous (TDO) syndrome or amelogenesis imperfecta.

### BRIEF DESCRIPTION OF THE DRAWINGS

FIG. 1A-1J shows a single cell atlas of the developing human fetal jaws, teeth, and salivary glands tissues via sci-RNA seq. Human tooth and salivary gland exhibit stepwise developmental processes (FIG. 1A). The oral epithelium will give rise to the epithelial components of teeth and salivary glands, while the condensed dental ectomesenchyme (DEM, colored in grey) will give rise to the mesenchymal component of these tissues. TG: toothgerm, DF: dental follicle, DP: dental papilla, P-de: pre-dentin, De: dentin, En: enamel matrix. (FIG. 1B) Human fetal tooth germs and salivary glands were dissected in a stage-specific manner from human fetal jaw tissue. The young fetal jaws, 9-11 gestational weeks were segmented into anterior segments (dotted box, which span from canine-to-canine region), and posterior jaw segment pairs (dotted box) and sequenced independently. For older fetal jaws, 1222 gestational weeks, individual toothgerms and submandibular salivary glands were dissected (FIG. 1C) Density plots of the clustered sci-RNA-seq data highlight the location of each tissue type in the same UMAP coordinate in FIG. 1D. The UMAP graph (FIG. 1D) yielded 20 annotated clusters from all sequenced data. (FIG. 1E) Immunofluorescence staining of developing toothgerms tissue sections with anti-Krt5 that specifically mark the dental epithelial morphology throughout the developmental stages. Counterstained with the nuclear staining DAPI. To establish expression of known odontoblast and ameloblast markers in the tissue, immunohistochemistry was performed on human fetal toothgerm at 20 gw using dentin sialophosphoprotein (DSPP) and ameloblastin (AMBN) respectively. As expected (FIG. 2B; FIG. 7A), all oral epithelium derived tissue was visualized by KRT5 (FIGS. 1F-1M), ameloblasts by AMBN (FIG. 1F-1I), and odontoblasts by DSPP (FIGS. 1J-1M). Mirrored expression patterns of AMBN and DSPP were observed between the ameloblast and odontoblast (FIGS. 1G-1H; FIGS. 3H, 3Q, 3Y) consistent with previous studies illustrating reciprocal expression between these two developing cell types. Scale bars: 50 μm.

FIGS. 2A-2K shows cell types identified by sciRNAseq are present at specific spatio-temporal stages of tooth development in vivo. (FIG. 2A) UMAP graph of subclustered molar and incisor tooth germ type dental mesenchyme derived cells from the total dataset identified 6 transcriptionally unique clusters including dental papilla (DP), preodontoblast (POB), odontoblast (OB), subodontoblast (SOB), odontoblast (OB), dental ectomesenchyme (DEM), and dental follicle (DF). (FIG. 2B) A custom heatmap was generated to identify the marker genes specific to each cluster, the top associated GO-terms to characterize cluster function, and calculated Age Score per cluster. (FIG. 2C) Pseudotime trajectory analysis for dental mesenchyme derived cells suggest two progenitors DP and DEM, that give rise to differentiated OB (yellow). (FIG. 2D) Real-time density plots indicate migration of cells from early progenitor populations DEM and DP at 9-16 gw to differentiated DF, POB, SOB and OB at later development 17-22 gw. (FIG. 2E) Simplified differentiation trajectory tree illustrating a common PRRX1+progenitor gives rise to both DP and DEM. In the OB lineage, DP gives rise to POB, followed by OB, with a suggested SOB transitioning through POB-like state before giving rise to OB; and DF lineage, of DEM giving rise to DF. (FIG. 2F) RNAscope Multiplex in situ for DEM (PRRX1+), DP (SOX5+FGF10+SA LL1+) and DF (IGFBP5+). (FIG. 2G) RNAscope map for marker combinations corresponding to individual dental mesenchyme clusters at 80d shown in aggregate in FIG. 2F (arrows indicate DF within the dental pulp). (FIG. 2H) RNAscope Multiplex in situ for OB (DSPP+), SOB (IGFBP5+SA LL1+), POB (FBN2+SA LL1+), and DF (IGFBP5+). (FIG. 2I) RNAscope map for marker combinations corresponding to individual dental esenchyme clusters at 117d shown in aggregate in FIG. 2H (SOB beneath OB at incisal edge (arrow) and intermingled with POB (arrow). (FIG. 2J) A diagram of the developing dental mesenchyme derived cell types of the human tooth germ. At cap stage (12-13 gw) the dental pulp consists of DP cells with DEM with sparse DF within; DF surrounds the developing toothgerm. (FIG. 2K) By bell stage (17-22 gw), the dental pulp consists of OB at the incisal edge, SOB and POB with small contributions of the DEM and DP. Dental epithelium derived enamel organ is indicated by KRT5 (FIGS. 2F and 2H; FIGS. 2G and 2I).

FIGS. 3A-3Y depicts spatial expression of odontoblast and ameloblast markers differs markedly from early to late toothgerm development. Ameloblast markers amelogenin (AME LX) and ameloblastin expression begins in the ameloblast after the early bell stage (FIGS. 3A-3J, FIGS. 3K-3R). Similarly, odontoblast marker dentin sialo phosphoprotein (DSPP) begins in the odontoblast after the early bell stage (FIGS. 3S-3Y). Heatmaps of expression over time of AME LX (FIG. 3E), AMBN (FIG. 30), and DSPP (FIG. 3W). AME LX, AMBN and DSPP show mirrored expression patterns in aneloblasts and odontoblasts at late bell stage (FIGS. 3H, 3Q, 3Y). Abbreviations: preodontoblast (POB), odontoblast (OB), preameloblast (PA) ameloblast (AM), incisal edge (IE), cervical loop (CL). Scale bars: 50 μm.

FIG. 4A-4D examines top signaling pathways of odontoblast differentiation trajectory. (FIG. 4A) Downstream signaling pathways ranked by activity in odontoblast differentiation indicate FGF and BMP are critical to the DP as it transitions to POB; HH, BMP and NOTCH are the most active as POB transitions to OB. (FIG. 4B) Diagrams illustrate the dental epithelium and ectomesenchyme derived cells present during early tooth development (9-16 gw) and late tooth development (17-22 gw), and the suggested ligand sources for each pathway during the transition from DP to POB and POB to OB. Note that the far right image is an inset of the whole toothgerm focused on the incisal edge. The majority of FGF and BMP signaling ligands are produced by the dental epithelium derived EK and IEE, with BMP ligands also produced by the dental ectomesenchyme derived DEM and DP during the transition from DP to POB. During the transition from POB to OB, the dental epithelium derived PA and AM produce much of the FGF, BMP and HH signaling ligands; a smaller contribution of FGF ligands is made by the SOB and autocrine signaling from the POB. (FIG. 4C) The sources of critical signaling ligands for the top pathways involved for each developmental stage originate from both the dental epithelium and mesenchyme derived tissues, with the thickness of the line indicating the number of ligand: receptor interactions, arrowheads indicating the cell possessing the receptor, and interactions of interest (red) and between support cells (black). (FIG. 4D) Heatmaps for the top pathways were generated by aggregating pathway ligand gene expression, which is then averaged per cluster.

FIG. 5A-5I examines HiPSC-derived odontoblast differentiation guided by sci-RNA-seq (iOB) produces mature odontoblast cells. (FIG. 5A) Schematic of the 11-day neural crest differentiation protocol (iNC) as described previously (Studer). (FIG. 5B) 98% of cell differentiated towards neural crest fate express neural crest marker p75 (CD271) as assessed by magnetic cell sorting. (FIG. 5C) Immunofluorescence staining of iNC show expression of neural crest markers p75 in the cytoplasm and AP-2a localized to the nucleus. (FIG. 5D) iNC show upregulated expression of neural crest markers PAX3 and SOX10 as assessed by QRT-PCR compared to undifferentiated HiPSC control. Each study was performed in triplicate, with error bars representing SEM. (FIG. 5E) Schematic of the 15-day differentiation protocol produced, which targets the identified signalling pathways utilizing growth factors and small molecules to transition through the odontoblast developmental trajectory. iNC cultured in odontogenic media and treated with FGFR superagonist minibinder (iOB) show decreased expression of neural crest markers PAX3 and SOX10 (FIG. 5F) and upregulated expression of odontoblast markers MSX1, DSPP and S100A13 (FIG. 5G) as assessed by QRT-PCR compared to undifferentiated HiPSC control. Each study was performed in triplicate, with error bars representing SEM. (FIG. 5H) Immunofluorescence staining of iOB shows DSPP present in the cytoplasm or secreted from the cell. (FIG. 5I) HiPSC and iOB were stained for extracellular calcifications with Alizarin Red Stain. Spectrometric quantification of Alizarin stain normalized to HiPSC control shows iOB have enhanced mineralization capacity. *p<0.05; **p<0.01, ***p<0.001.

FIG. 6A-6I shows loss of disease associated transcription factor DL X3 inhibits odontoblast maturity in vitro. (FIG. 6A) Sanger sequencing results of DLX3 knock-out mutant line compared to HiPSC shows initial 54% of cells possess a single base pair deletion at site 374 with removal of a single glycine nucleotide (SEQ ID NO: 1, SEQ ID NO: 2, SEQ ID NO: 3, and SEQ ID NO: 4)). This population transitions to 84% after odontoblast differentiation (FIG. 6B). Solid black line indicates guide RNA (gRNA) sequence; black dotted line indicates cut site; red dotted line indicates PAM sequence (SEQ ID NO:5, SEQ ID NO: 6). (FIG. 6C) Presence of D LX3 protein was evaluated by Western Blot, showing loss of protein in the mutant line. (FIG. 6D) Mutant cells were differentiated towards neural crest fate as described previously and showed the same 98% efficiency of producing p75+ cells during magnetic cell sorting. DLX3 mutants show unchanged expression of neural crest markers PAX3 and SOX10 compared to iNC (FIG. 6E) with significantly decreased expression of odontoblast marker DSPP (FIG. 6F) compared to iOB as assessed by QRT-PCR. Each study was performed in triplicate, with error bars representing SEM. The loss of DLX3 on mineralization capacity was evaluated by Alizarin Red Staining (FIG. 6G). Spectrometric quantification of Alizarin stain normalized to HiPSC control shows DLX3 mutants have mineralization capacity similar to control (FIG. 6H). (FIG. 6I) DLX3 expression in human dental ectomesenchyme derived cells overtime. *p<0.01; ***p<0.001.

FIG. 7A-7G shows expression of known marker genes for dental ectomesenchyme derived cell types. (FIG. 7A). Heatmap of known marker gens for dental ectomesenchyme derived cell types. (FIG. 7B). Heatmap of expression overtime of dental follicle marker IGFBP5 and subodontoblast markers SALL1 (FIG. 7C). Gene density plot of shared DP and DEM progenitor marker PRRX1 (FIG. 7D). Cell cycle scoring of dental mesenchyme derived cell types. Mappings for dental mesenchyme-derived cell types at 80d replicate (FIG. 7E) and 117d replicate (FIG. 7F) identified by analysis of RNAscope images which show mapping for SOB, DF, DEM, OB, and POB cell types. (FIG. 7G) Top marker genes for dental ectomesenchyme derived cell types.

FIGS. 8A-8N examines individual channels of merged RNAScope images in FIG. 2F and H. FIGS. 8A and 8H) SOX5; FIGS. 8B and 8I) FGF10; FIGS. 8C and 8J) PRRX1; FIGS. 8D and 8K) SALL1; FIGS. 8E and 8L) FBN2; FIGS. 8F and 8M) IGFBP5; FIGS. 8G and 8N) DSPP in 80d and 117d incisor toothgerms respectively. Scale bar=100 μm.

FIG. 9A-9D examines individual channels of mapped RNAScope replicate images in FIGS. 2F and H. Mappings for dental mesenchyme-derived cell types at 80d replicate (FIGS. 9A and 9B) and 117d replicate (FIGS. 9C and 9D) identified by analysis of RNAscope images which show mapping for SOB, DF, DEM, OB, and POB cell types.

FIG. 10A-10E shows expression of DSPP protein and mineralization capacity of various odontoblast treatments. (FIG. 10A) Schematic of the 15-day differentiation protocol produced, which targets the identified signaling pathways utilizing growth factors and small molecules to transition through the odontoblast developmental trajectory. Treatments activated FGF signaling by supplementation of media with either FGFR superagonist minibinder, basic FGF (bFGF or FGF2), FGF8b; inhibited FGF signaling by supplementation of media with FGFR antagonist minibinder; or were not treated with FGF. (FIG. 10B) Immunofluorescence staining of treated cells shows DSPP present in cells treated with FGFR superagonist. (FIG. 10C) Quantification of DSPP integrated density normalized to DAPI shows a two-fold increase in DSPP expression in cells treated with FGFR superagonist compared to those treated with bFGF. (FIG. 10D) Differentiated cells were stained for extracellular calcifications with Alizarin Red Stain. (FIG. 10E) Spectrometric quantification of Alizarin stain normalized to HiPSC control shows iOB treated with FGFR superagonist have significantly increased mineralization capacity. p<0.01; ***p<0.001.

FIG. 11A-11J shows single cell RNA sequencing of fetal tooth germ predicts FGF, BMP, and HH signaling are critical to human odontoblast development. (FIG. 11A) Downstream signaling pathways ranked by activity in odontoblast development indicate FGF and BMP are critical to the dental papilla (DP) as it transitions to preodontoblast (POB); HH, BMP and NOTCH are the most active as POB transitions to odontoblast (OB). The sources of critical signaling ligands for the top pathways involved for DP to POB (FIG. 11B) and POB to OB (FIG. 11C) originate from both the dental epithelium and mesenchyme derived tissues. The number of ligand-receptor interactions denoted by the thickness of the line, arrowheads indicating the cell possessing the receptor, and interactions of interest and between support cells, with the progenitor of interest in the red box. Heatmaps were generated by aggregating pathway ligand and receptor gene expression for the DP to POB (FIGS. 11D, 11F) and POB to OB (FIGS. 11E,11G) transitions, averaged per cluster. (FIG. 11H) Summary schematic depicting early human tooth development, where it is predicted that the majority of FGF and BMP signaling ligands are produced by the dental epithelium derived enamel knot (EK) and inner enamel epithelium (IEE), respectively, which bind to receptors on the DP. (FIG. 11I) At late human tooth development, the dental epithelium derived preameloblast (PA) and ameloblast (AM) produce much of the FGF, BMP and HH signaling ligands and bind to receptors on the POB (FIG. 11J). The color of arrow denotes expression of signaling ligands and receptors. Note that this image is an inset of the whole tooth germ focused on the incisal edge. Abbreviations: non-canonical WNT (ncWNT). Graphic generated using InkScape.

FIG. 12A-12F shows human molar tooth development is delayed compared to incisors. The proportion of each cell type present within the dental ectomesenchyme (FIG. 12A) and dental epithelium (FIG. 12B) derived cells. Developmental scores calculated to compare differentiation states in the odontoblast (FIG. 12C) and ameloblast (FIG. 12D) trajectory. Gestational week at which odontoblasts (FIG. 12E) and ameloblasts (FIG. 12F) first appear. Graphic generated using Biorender.

FIGS. 13A-13P examines dental ectomesenchyme derived cells are the primary source of signaling ligands in enamel knot and ameloblast development regardless of tooth type. (FIG. 13A) Downstream signaling pathways ranked by activity in the differentiation of the dental epithelium (DE) to enamel knot (EK) segmented by incisor and molar tooth germ type indicates FGF and WNT signaling is critical in the incisor while molars required BMP and ROBO signaling. The sources of critical signaling ligands for the pathways involved in DE to EK transition originate from the dental ectomesenchyme derived tissues in both the incisor (FIG. 13B) and molar (FIG. 13C). The number of ligand-receptor interactions denoted by the thickness of the line, arrowheads indicating the cell possessing the receptor, and interactions of interest and between support cells, with the progenitor of interest in the red box. Heatmaps were generated by aggregating pathway ligand (FIG. 13D) and receptor (FIG. 13E) gene expression, averaged per cluster. Summary schematics illustrate the dental epithelium and ectomesenchyme derived cells present during the transition from DE to EK. Elevated BMP signaling ligand production by the dental ectomesenchyme (DEM) is observed in the molar, with increased BMPR's on the DE. Increased SLIT ligand and ROBO receptor expression is seen in the molar DE (FIG. 13F). Comparatively, in the incisor, FGF and WNT signaling appears to play a critical role in differentiation of DE to EK (FIG. 13G). Downstream signaling pathways ranked by activity in the differentiation of the dental epithelium (DE) to outer enamel epithelium (OEE) followed by preameloblast (PA) segmented by incisor or molar tooth germ type indicates incisor tooth germs have increased FGF and EGF signaling during the transition from DE to OEE, while molars require robust FGF and BMP signaling during the maturation of OEE to PA in humans (FIG. 13H). The sources of critical signaling ligands for the pathways involved in ameloblast development originate from the dental ectomesenchyme derived tissues in both the incisor and molar in the transition from DE to OEE (FIG. 13I) and OEE to PA (FIG. 13J). The number of ligand-receptor interactions denoted by the thickness of the line, arrowheads indicating the cell possessing the receptor, and interactions of interest and between support cells, with the progenitor of interest in the red box. Heatmaps were generated by aggregating pathway ligand and receptor gene expression, averaged per cluster. During the transition from DE to OEE, the incisor DE is vastly activated by EGF ligands produced by the dental follicle (DF) and FGF ligands produced by the dental papilla (DP) (FIG. 13K), which bind to receptors on the DE (FIG. 13L). During the transition from OEE to PA, the molar OEE is vastly activated by BMP and FGF ligands produced by the DP (M), which bind to receptors on the OEE (FIG. 13N). Summary schematics illustrate the dental epithelium and ectomesenchyme derived cells present during the transition from DE to OEE to PA. These bioinformatics based predictions suggest that FGF and EGF signaling is critical for early ameloblast development in the incisor (FIG. 13P), while FGF and BMP are required for preameloblast maturation in the molar (FIG. 13O), and that the dental ectomesenchyme cells are largely responsible for secretion of the signaling ligands which activate these pathways. The arrow denotes expression of signaling ligands and receptors. Graphic generated using InkScape.

FIGS. 14A-14F show hiPSC successfully differentiate to neural crest fate (iNC). (FIG. 14A) Schematic of the 11-day neural crest differentiation protocol (iNC). (FIG. 14B) 90% of hiPSC differentiated towards neural crest fate express neural crest marker p75 (CD271) as assessed by magnetic cell sorting. Immunofluorescence staining of iNC show expression of neural crest markers p75 and AP-2α (FIG. 14C). Scale bar 10 μm. AP-2α is localized to the nucleus (FIG. 14D). Scale bar 14 μm. (FIGS. 14E and 14F) qPCR of neural crest markers PAX3 and NESTIN. Each study was performed in triplicate (N=3), with error bars representing standard error of the mean (SEM). ***p<0.001; **** p<0.00001.

FIGS. 15A-15L depict odontoblast differentiation guided by sci-RNA-Seq using the de novo designed FGFR1c mini binder C6 produces more mature odontoblasts with increased mineralization capacity. (FIG. 15A) Model of the de novo FGFR1/2c mini binder (hereby referred to as mb7) and cyclic, homo-oligomeric, hexameric scaffold fusing six mb7 (hereby referred to as C6) engaging six FGFR1/2c. (FIG. 15B) 25-day iOB differentiation protocol, which first transitions through iNC before targeting the sci-RNA-seq identified signaling pathways FGF, BMP and HH to produce mature odontoblasts. (FIG. 15C) Schematic of the iOB differentiation protocol where iNC are cultured in odontogenic medium (OB); supplemented with BMP4 and SAG (iOB); C6 (iOB C6); C6 followed by mb7 (iOB C6 to mb7); or recombinant basic FGF (iOB bFGF). (FIG. 15D) Western blot analysis of NESTIN, RUNX2 and DSPP. (FIG. 15E) Quantification of DSPP protein levels. (FIG. 15F) Immunofluorescence staining of odontoblast markers DSPP and RUNX2 with white arrows indicating DSPP and RUNX2. Scale bar 50 μm. qPCR analysis of odontoblast markers DSPP (FIG. 15G), DMP1 (FIG. 15H) and FGFR1c (FIG. 15I) expression. Cells stained for extracellular calcifications with Alizarin Red Stain (ARS) (FIG. 15J). Spectrometric quantification of ARS normalized to hiPSC control (FIG. 15K). Higher magnification image of ARS and calcified nodule formation (FIG. 15L). Scale bar 20 μm. Each study was performed in triplicate (N=3), with error bars representing standard error of the mean (SEM). *p<0.05; ** p<0.01; **** p<0.001.

FIG. 16A-16O shows a Single Cell Atlas of the Developing Human Incisor and Molar Dental Cell Types. (FIG. 16A) Downstream signaling pathways ranked by activity with detailed signaling ligands per pathway in odontoblast development indicate FGF and BMP are critical to the dental papilla (DP) as it transitions to preodontoblast (POB); HH, BMP and NOTCH are the most active as POB transitions to odontoblast (OB). UMAP graph of subclustered incisor (FIG. 16B) and molar (FIG. 16E) tooth germ type dental mesenchyme derived cells from the total dataset identified conserved 6 transcriptionally unique clusters identified by collating highly expressed cluster-specific genes including dental papilla (DP), preodontoblast (POB), odontoblast (OB), subodontoblast (SOB), odontoblast (OB), dental ectomesenchyme (DEM), and dental follicle (DF). Pseudotime trajectory analysis for dental mesenchyme derived cells suggest two progenitors DP and DEM, that give rise to differentiated OB for both incisor (FIG. 16C) and molar (FIG. 16F). Heatmaps for putative marker genes for each dental mesenchyme cell type were produced for incisor (FIG. 16D) and molar (FIG. 16G). Simplified differentiation trajectory tree illustrating a common DEM progenitor gives rise to both DP and DF. In the OB lineage, DP gives rise to POB, followed by OB; DF lineage indicates DEM giving rise to DF, which gives rise to SOB, with a suggested transition through POB-like state before giving rise to OB (H). UMAP graph of subclustered incisor (FIG. 16I) and molar (FIG. 16L) tooth germ type dental epithelium derived cells from the total dataset yielded 12 unique clusters that we identified by collating highly expressed cluster-specific genes including: oral epithelium (OE), dental epithelium (DE), enamel knot (EK), inner and outer enamel epithelium (IEE, OEE), cervical loop (CL), inner and outer stratum intermedium (SII, SIO), inner and outer stellate reticulum (SRI, SRO), pre-ameloblasts (PA) and ameloblast (AM). Pseudotime trajectory analysis for dental epithelium derived cells suggests the OE directly gives rise to DE, followed by EK. The DE also gives rise to the SR and OEE lineages, which give rise to SI, IEE/PA, and AM in both the incisor (FIG. 16J) and molar (FIG. 16M). Heatmaps for putative marker genes for each dental epithelium cell type were produced for both incisor (FIG. 16K) and molar (FIG. 16N). Simplified differentiation trajectory tree illustrating a common DE progenitor gives rise to EK, SR, and OEE lineages (FIG. 16O).

FIG. 17A-17H examine C-isoform specific activation of FGFR1 with de novo Designed Mini Binder C6 Promotes Improved Mineralization Capacity of iOB. Quantification of Western Blot protein level of RUNX2 (FIG. 17A) and NESTIN (FIG. 17B). qPCR analysis of odontoblast marker RUNX2 (FIG. 17C). Extracellular calcifications assessed via Alizarin Red Stain (ARS) in iNC cultured in odontogenic medium (OB) (FIG. 17D); supplemented with BMP4 and SAG (iOB) (FIG. 17E); C6 (iOB C6) (FIG. 17F); C6 followed by mb7 (iOB C6 to mb7) (FIG. 17G); or undifferentiated hiPSC (FIG. 17H). Scale bar 20 μm.

FIG. 18 shows bulk RNA-seq analysis demonstrating upregulation of NC markers TFAP2A, PAX3, NES, and PAX7 at Day 11 of iNC differentiation compared to undifferentiated hiPSC control.

FIG. 19 analyzes pathway enrichment of the Bulk-seq dataset. Functional enrichment showing the 30 most significant categories of molecular functions (GO terms) of iNC samples performed with ShinyGO software v0.741.

FIG. 20 shows bulk RNA-seq analysis demonstrating upregulation of OB markers: VIM, MSX1, NES, and BACH1 at Day 25 of iOB differentiation compared to undifferentiated hiPSC control.

FIG. 21 shows bulk RNA-seq analysis demonstrating upregulation of OB markers: IGF2, PHEX, RUNX2, MSX1, LAMA2 and LAMA3 at Day 25 of iOB_C6 differentiation compared to undifferentiated hiPSC control.

FIG. 22 shows bulk RNA-seq analysis demonstrating upregulation of OB markers VIM, MSX1, NES, and BACH1 at Day 25 of iOB differentiation compared to iNCs.

FIG. 23 shows bulk RNA-seq analysis demonstrating upregulation of OB markers IGF2, PHEX, RUNX2, MSX1, LAMA2 and LAMA3 at Day 25 of iOB_C6 differentiation compared to iNCs.

FIG. 24 analyzes pathway enrichment analysis of the Bulk-seq dataset. Functional enrichment showing the 30 most significant categories of molecular functions (GO terms) of iOB samples performed with ShinyGO software v0.741.

FIG. 25 shows bulk RNA-seq analysis demonstrating upregulation of OB markers: IGF2, PHEX, IGFBP5, SERPINF1, SORCS2, PTGES, and RUNX2 at Day 25 of iOB_C6 differentiation compared to differentiated iOB samples.

FIG. 26 shows pathway enrichment analysis of the Bulk-seq dataset. Functional enrichment showing the 30 most significant categories of molecular functions (GO terms) of iOB_C6 samples performed with ShinyGO software v0.741.

FIG. 27 analyzes pathway enrichment analysis of the Bulk-seq dataset. Functional enrichment showing the 20 most significant categories of biological processes (GO terms) of iOB_C6 samples performed with ShinyGO software v0.741.

FIG. 28 shows a heatmap of expression over time of pluripotent markers (day-3 to day 0), neural crest markers (day 0 to day 11) and odontoblast markers (day 11 to day 25) for hiPSCs, iNCs, iOB and iOB_C6 samples.

FIG. 29 shows a schematic for the generation of DLX3 knockout mutant for Tricho-Dento-Osseous Disease modeling.

FIG. 30 shows a schematic of how to analyze top pathways identified between the progenitor and the differentiated cell.

FIG. 31 examines the loss of DLX3 arrests odontoblast maturity at the preodontoblast stage in vitro.

FIG. 32 shows the deletion of Cys374 in DLX3 leads to a frame shift and early stop codon.

FIG. 33 shows comparisons focusing on the overlap between enriched marker genes of fetal OB population with hiPSCs derived iOB_C6 datasets. To assess the validity of the proposed clusters in the fetal OB lineage, datasets were compared and hiPSCs derived iOB_C6 datasets identified the enriched marker genes for each dataset and found significant overlaps between human fetal OB and hiPSCs derived iOB_C6 cell population.

### DETAILED DESCRIPTION

The methods and compositions described herein are based, in part, on the discovery of methods for generating odontoblasts or organoids that comprise them, in vitro from pluripotent stem cells. Also provided herein are compositions comprising in vitro-derived odontoblasts for administration or transplantation into a subject to induce dentin production to treat structural tooth defects, dentin loss or demineralization. Also provided herein, are products produced using the odontoblast or odontoblast organoids produced as described herein.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “contacting” when used in reference to a cell, encompasses both introducing an agent, growth factor, surface, etc. to the cell in a manner that permits physical contact of the cell with the agent, growth factor, surface, etc., and introducing an element, such as a genetic construct or vector, that permits the expression of an agent, such as a polypeptide, or other expression product in the cell.

The terms “stem cell” or “undifferentiated cell” as used herein, refer to a cell in an undifferentiated or partially differentiated state that has the property of self-renewal and has the developmental potential to differentiate into one or more additional cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is referred to as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also potentially retaining one or more cells with parental developmental potential. A differentiated cell may derive from a multipotent cell, which itself is derived from a multipotent cell, and so on. While each of these multipotent cells can be considered stem cells, the range of cell types each such stem cell can give rise to, i.e., their developmental potential, can vary considerably. Alternatively, some of the stem cells in a population can divide symmetrically into two stem cells, referred to as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term “stem cell” refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art, and as used herein.

Exemplary stem cells include embryonic stem cells, adult stem cells, pluripotent stem cells, bone marrow stem cells, mesenchymal stem cells, hematopoietic stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735.

In the context of cell ontogeny, the term “differentiate”, or “differentiating” is a relative term that indicates a “differentiated cell” is a cell that has progressed further down a developmental pathway than its precursor cell. Thus, in some embodiments, a stem cell as the term is defined herein, can differentiate to lineage-restricted precursor cells, which in turn can differentiate into other types of precursor cells further down the pathway, and then to an end-stage differentiated cell, which plays a characteristic role in a certain tissue type, and may or may not retain the capacity to proliferate further. The differentiation status of a cell is generally determined by one or more of characteristic gene or marker expression pattern, metabolic activit(ies), and morphology.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “iPSC” and “induced pluripotent stem cell” are used interchangeably and refer to a pluripotent stem cell artificially derived from a differentiated somatic cell. iPSC cells are capable of self-renewal and differentiation into cell fate-committed stem cells, including cells associated with tooth development, as well as various types of mature cells. As used herein, the term hiPSC refers to iPSCs derived from human somatic cells.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell.

A “marker” as used herein refers to one or more characteristics that contribute to or are associated with the phenotype of a given cell. Markers can be used for selection of cells comprising characteristics of interest. Markers will vary with specific cells, and can be characteristics, whether morphological, functional or biochemical, that are particular to a cell or tissue type, or to a disease state or phenotype and include both extracellular and intracellular molecules, e.g., proteins, RNAs, glycosylation patterns, etc. expressed or exhibited by the cell or tissue. In some embodiments, such markers are proteins, including, but not limited to proteins that possess an epitope for an antibody or other binding molecule available in the art. Other markers can include peptides, lipids, polysaccharides, nucleic acids and steroids, among others. Examples of morphological characteristics or traits include, but are not limited to, shape, size, and nuclear to cytoplasmic ratio. Examples of functional characteristics or traits include, but are not limited to, the ability to produce dentin or dental enamel. Markers can be detected by any appropriate method available to one of skill in the art.

The terms “decrease,” “reduced,” “reduction,” “decrease,” or “inhibit” as used herein generally refer to a decrease by a statistically significant amount. However, for avoidance of doubt, “reduced”, “reduction” or “decrease” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased,” “increase,” or “enhance,” or “activate” as used herein generally refer to an increase by a statically significant amount. However, for the avoidance of doubt, the terms “increased”, “increase” or “enhance” or “activate” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase or more, relative to a reference level.

As used herein, the term “modulates” refers to an effect including increasing or decreasing a given parameter as those terms are defined herein.

As used herein, the term “organoid” refers to a 3D multicellular tissue culture construct comprising cells differentiated in vitro from stem cells, including, but not limited to iPS cells. Organoids can, but need not necessarily include a plurality of different cell types. In one embodiment, an organoid comprising odontoblasts, e.g., differentiated as described herein, comprises a spherical arrangement of odontoblasts and extracellular matrix in which the cells are arranged and oriented so as to secrete dentin sialophosphoprotein into a lumen of the organoid.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The technology described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Pluripotent Stem Cells

In the methods described herein, odontoblasts are differentiated in vitro from pluripotent stem cells. The pluripotent stem cells can be, for example, embryonic stem cells, which, as the name indicates, are isolated from a developing embryo, or so-called induced pluripotent stem cells, which are isolated from somatic cells by reprogramming.

Embryonic Stem Cells: Embryonic stem (ES) cells and methods for their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435), among other references. Embryonic stem cells are the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). Markers characteristic of ES cells are known in the art and discussed in these references. Undifferentiated ES cells are easily recognized by those skilled in the art, and typically appear in the two dimensions of a microscopic view as colonies of cells with high nuclear/cytoplasmic ratios and prominent nucleoli. In some embodiments, the odontoblasts described herein are not derived from embryonic stem cells or any other cells of embryonic origin.

Induced Pluripotent Stem Cells: In some embodiments, odontoblasts as described herein are differentiated from induced pluripotent stem cells (iPSCs). Although differentiation is generally irreversible under physiological contexts, methods have been developed in recent years to reprogram somatic cells to induced pluripotent stem cells. The earliest approaches used retroviral transduction of a set of factors, cOMyc, Oct3/4, Sox2 and Klf4, now known as the “Yamanaka factors,” to reprogram somatic cells (fibroblasts, originally) to a pluripotent phenotype (See, e.g., Takahashi & Yamanaka, Cell 126: 663-676 (2006)). Since then, numerous improvements and variations on the approach have used different combinations of these and other reprogramming factors to achieve the same pluripotent state with a number of different somatic cell sources. Variations on the method that use non-retroviral or non-viral transduction of DNA encoding the factors, mRNA encoding the factors, and even small molecules that mimic the reprogramming factors are known in the art; see, e.g., Wernig et al., Nature 448: 318-324 (2007), Stadtfeld et al., Science 322: 945-949 (2008), Yoshioka et al., Cell Stern Cell 13: 10.1016/j.stem.2013.06.001; Warren & Lin, Mol. Ther. 27: 729-734 (2019); Zhou et al., Cell Stem Cell 4: 381-384 (2009), each of which is incorporated herein by reference. Without limitations, iPS cells can also be generated using other methods, including, but not limited to non-viral methods, use of polycistronic vectors, mRNA species, miRNAs, and proteins, including methods described in, for example, International Patent Applications WO2010/019569, WO2009/149233, WO2009/093022, WO2010/022194, WO2009/101084, WO2008/038148, WO2010/059806, WO2010/057614, WO2010/056831, WO2010/050626, WO2010/033906, WO2009/126250, WO2009/143421, WO2009/140655, WO2009/133971, WO2009/101407, WO2009/091659, WO2009/086425, WO2009/079007, WO2009/058413, WO2009/032456, WO2009/032194, WO2008/103462, JP4411362, EP2128245, and U.S. Patent Applications US2004/0072343, US2009/0253203, US2010/0112693, US2010/07542, US2009/0246875, US2009/0203141, US2010/00625343, US2009/0269763, and US2010/059806, each of which are incorporated herein in their entirety by reference. While any tissue can provide source cells for generating iPS cells to differentiate into odontoblasts, it is contemplated that cells derived from dental gum tissue may have benefits, for example, epigenetic memory, that permit enhanced generation of mature ameloblasts when used in the methods described herein to generate iPS cell-derived odontoblasts.

In some embodiments, the methods and compositions described herein use odontoblasts and other cells (e.g., ameloblasts) differentiated in vitro from iPS cells. An advantage of using iPSCs to generate odontoblasts or other cells for the compositions described herein is that the cells can be derived from the same subject to which the desired human odontoblasts are to be administered. That is, a somatic cell can be obtained from a subject, reprogrammed to an induced pluripotent stem cell, and then re-differentiated into a human odontoblast or other cell to be administered to that subject (e.g., autologous cells). Since the odontoblasts or other cells (or their differentiated progeny) are essentially derived from an autologous source, the risk of engraftment rejection or allergic responses is reduced compared to the use of cells from another subject or group of subjects. In some embodiments, the odontoblasts or other cells useful for the compositions described herein are derived from non-autologous sources. In addition, the use of iPSCs negates the need for cells obtained from an embryonic source. Thus, in one embodiment, the stem cells used to generate odontoblasts or other cells for use in the compositions and methods described herein are not embryonic stem cells.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3: 132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

The specific approach or method used to generate pluripotent stem cells from somatic cells (e.g., any cell of the body with the exclusion of a germ line cell) is not critical to the claimed invention. Thus, any method that re-programs a somatic cell to the pluripotent phenotype can be appropriate for use in the methods described herein.

### Preparation of Stem Cell-Derived Odontoblasts

As used herein, an odontoblast is a cell of neural crest origin that is a part of the outer surface of the dental pulp. It forms dentin, the substance beneath the tooth enamel on the crown and the cementum on the root. Odontoblasts can first appear at sites of tooth development at 17-18 weeks in utero and remain present until death unless killed by bacterial or chemical attack, or indirectly through other means such as heat or trauma (e.g. during dental procedures). Markers expressed by odontoblasts include, but are not limited to dentin matrix acidic phosphoprotein 1 (DMP-1) and dentin sialophosphoprotein (DSPP).

Provided herein are methods for generating odontoblasts from pluripotent stem cells. As noted above, the pluripotent stem cells can be embryonic stem cells or iPS cells. Due in part to the advantages noted above, iPS cells will likely be used most often. Also described herein are methods of producing stem cell-derived odontoblasts beginning with somatic cells derived from a subject, patient or donor, including a subject having a given disease or disorder that affects tooth structure. In these instances, the somatic cells are reprogrammed to induced pluripotent stem cells (iPS cells, iPSCs), which are then differentiated to odontoblasts or organoids comprising odontoblasts or odontoblasts and ameloblasts.

In some embodiments, the differentiated cell can be an epithelial cell (e.g., an epithelial keratinocyte, such as an oral epithelial keratinocyte, or an epithelial cell of the skin). Oral epithelial cells can be harvested from any area of the oral cavity in which they reside (e.g., the palate). In some embodiments, the cell can be immortalized and/or genetically modified.

In some embodiments, the odontoblasts or odontoblast organoids described herein can be generated from enamel organ epithelial (EOE) cells or cell lines thereof. As noted, the cells can be immortalized, and they can in that instance be, for example, the immortalized EOE cell line generated by DenBesten et al., PABSo-E (DenBesten et al., Eur. J. Oral Sci., 107(4):276-81, 1999). Another useful cell type is the Epithelial Cell Rests of Malassez (ERM cells; see Shinmura et al., J. Cellular Physiol. 217:728-738, 2008). ERM cells can be obtained from periodontal ligament tissue by explant culture, sub-cultured with non-serum medium, and expanded on 3T3-12 feeder cell layers. The odontoblasts or organoids thereof can also be generated from skin epithelial cells (see Liu et al., J. Tissue Eng. Regen. Med. 7:934-943, 2012).

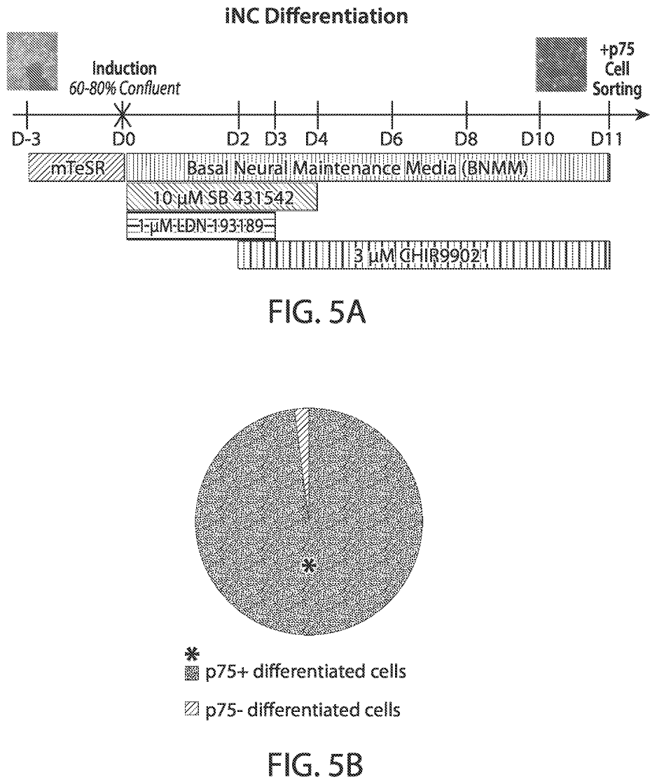

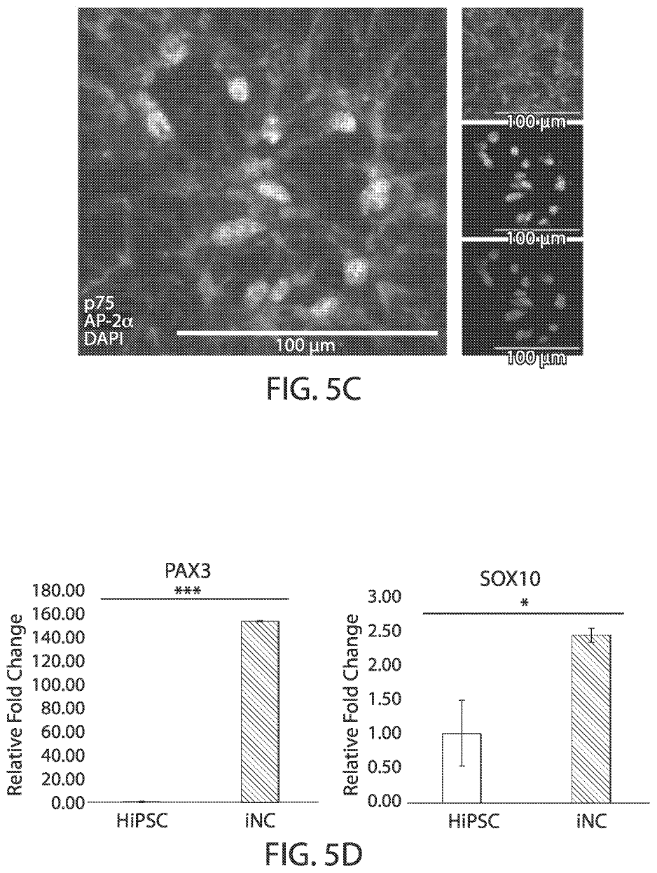

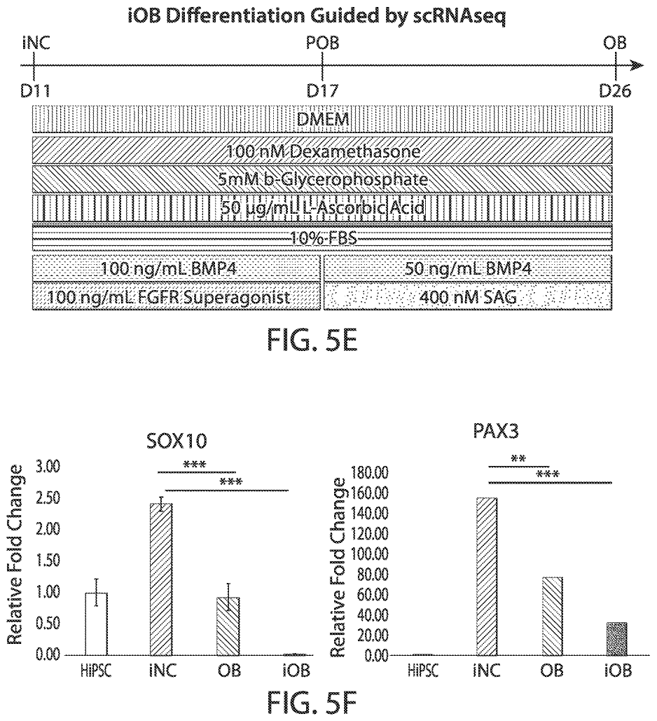

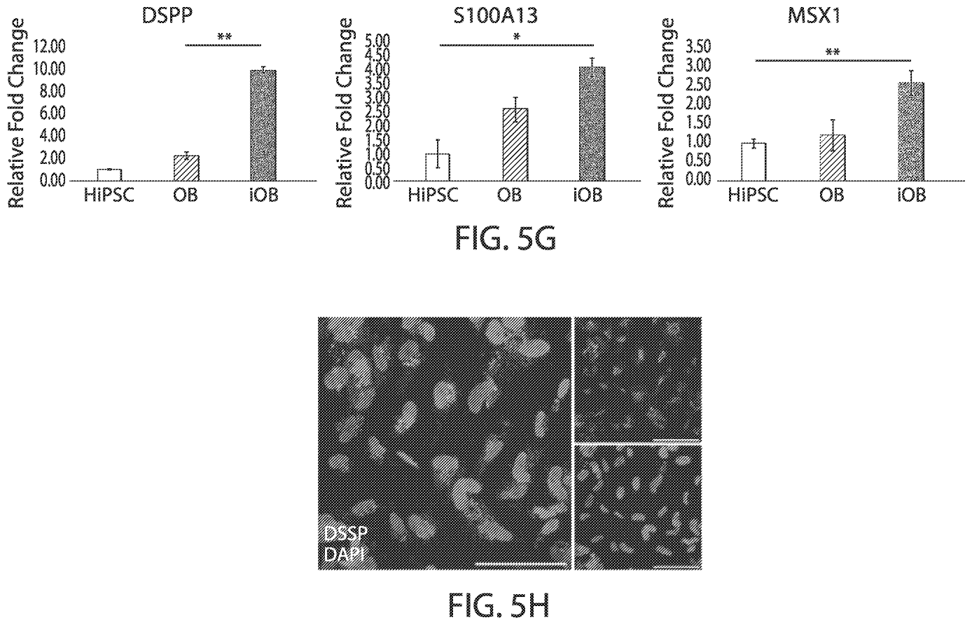

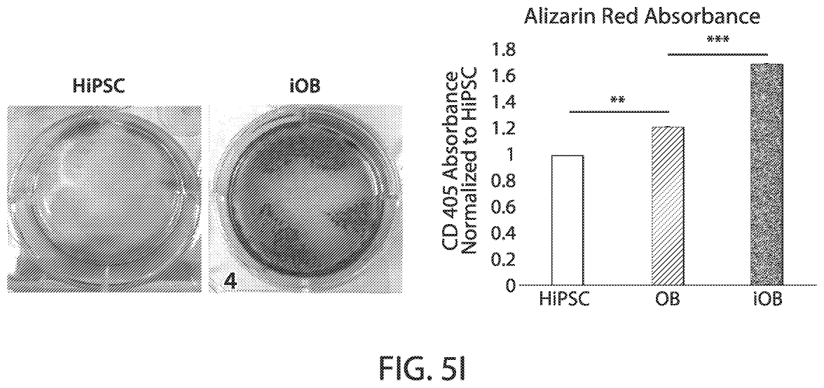

In one aspect, odontoblasts can be derived by in vitro differentiation from a pluripotent stem cell in a method having the following steps, in order:
a) contacting pluripotent stem cells at day zero with a TGF-β/SMAD inhibitor;
b) adding a WNT activator to the culture in (a) at day 2;
c) enriching the culture of step (b) for a population of induced neural crest stem cells;
d) contacting the population of induced neural crest stem cells enriched in (c) with an odontoblast differentiation medium comprising a BMP pathway agonist, an FGF pathway agonist, and a Hedgehog pathway agonist, and incubating to generate a population of odontoblast cells characterized by at least the expression of dentin sialophosphoprotein (DSPP). An exemplary embodiment of the method is demonstrated in the Examples provided herein. An exemplary timeline for the various agent treatments and procedures is shown in FIGS. 5a and 5e provided herein. Various considerations for performing this method are also discussed in the following.

TGF-β SMAD inhibitors: The production of neural crest stem cells from iPS cells involves inhibition of TGF-β signalling, e.g., via inhibitors of SMADs, intracellular factors that transduce TGF-β signals to the nucleus. TGF-β signaling pathway modulation: In some embodiments, one or more TGF-β antagonists are used to promote a particular differentiation step in a process of generating a desired cell type from a stem cell in vitro (e.g., from an iPS cell). In such embodiments, an inhibiting agent specific for TGF-β signaling can be a small molecule inhibitor, an antibody, or a ligand-binding antagonist. Examples of inhibiting agents specific for TGF-β signaling include, but are not limited to SB431542, LDN-193189, Galunisertib, LY2109761, SB5255334, SB505124, GW788388, LY364947, RepSox, LDN-193189 2HCl, K02288, LDN-214117, SD-208, Vactosertib, ML347, LDN-212854, DMH1, Pirfenidone, Alantolactone, SIS3HCl, and Hesperetin. In some embodiments, the TGF-β/SMAD antagonists are SB431542 and LDN-193189.

The Transforming Growth Factor Beta (TGF-β) signaling pathway is involved in many cellular processes in both the adult organism and the developing embryo including cell growth, cell differentiation, apoptosis, cellular homeostasis and other cellular functions. TGF-β superfamily ligands bind to a type II receptor, which recruits and phosphorylates a type I receptor. The type I receptor then phosphorylates receptor-regulated SMADs (R-SMADs) which then bind the coSMAD SMAD4. R-SMAD/coSMAD complexes accumulate in the nucleus where they act as transcription factors and participate in the regulation of target gene expression.

TGF-β1 is a prototypic member of a family of cytokines including the TGF-βs, activins, inhibins, bone morphogenetic proteins and Mullerian-inhibiting substance. Smad proteins are exemplary downstream signal transduction factors in the TGF-β pathway and therefore, in some embodiments, can be inhibited to promote differentiation of iPS cells to a neural crest phenotype useful for further differentiation to an odontoblast phenotype as described herein. As shown in the Examples below, SMAD inhibitors including, but not limited to SB431542 and LDN193189 can participate in promoting differentiation of iPS cells to the induced neural crest cell state.

In some embodiments, the dosage range useful for an antagonist of TGF-β/SMAD signaling is between 0.1 μM and 100 μM, for example, between 0.1 μM and 100 μM, between 0.1 μM and 90 μM, between 0.1 μM and 80 μM, between 0.1 μM and 70 μM, between 0.1 μM and 60 μM, between 0.1 μM and 50 μM, between 0.1 μM and 40 μM, between 0.1 μM and 30 μM, between 0.1 μM and 20 μM, between 0.1 μM and 10 μM, between 0.1 μM and 1 μM, between 1 μM and 100 μM, between 10 μM and 100 μM, between 20 μM and 100 μM, between 30 μM and 100 μM, between 40 μM and 100 μM, between 50 μM and 100 μM, between 60 μM and 100 μM, between 70 μM and 100 μM, between 80 μM and 100 μM, between 90 μM and 100 μM, between 95 μM and 100 μM or any range therebetween.

WNT activators agonists: Activators or agonists of Wnt signalling are used, in combination with other factors as described herein, to promote differentiation of iPS cells to the neural crest stem cell lineage prior to differentiating those cells to odontoblasts. Without wishing to be bound by theory, Wnt proteins and their cognate receptors signal through at least two distinct intracellular pathways. The “canonical” Wnt signaling pathway, (referred to herein as the Wnt/β-catenin pathway) involves Wnt signaling via β-catenin to activate transcription through TCF-related proteins (van de Wetering et al. (2002) Cell 109 Suppl: S13-9; Moon et al. (2002) Science 296(5573): 1644-6). A non-canonical alternative pathway exists, in which Wnt activates protein kinase C (PKC), calcium/calmodulin-dependent kinase II (CaMKII), JNK and Rho-GTPases (Veeman et al. (2003) Dev Cell 5(3): 367-77), and is often involved in the control of cell polarity.

As used herein, the term “Wnt agonist” or “Wnt pathway agonist” refers to any agent that activates the Wnt/β-catenin pathway, or inhibits the activity and/or expression of inhibitors of Wnt/β-catenin signaling, for example antagonists or inhibitors of GSK-3β activity. A Wnt activating agent as used herein can enhance signaling through the Wnt/β-catenin pathway at any point along the pathway, for example, but not limited to increasing the expression and/or activity of Wnt, or β-catenin or Wnt dependent genes and/or proteins, and decreasing the expression and/or activity of endogenous inhibitors of Wnt and/or β-catenin or decreasing the expression and/or activity of endogenous inhibitors of components of the Wnt/β-catenin pathway, for example decreasing the expression of GSK-3β. Some non-limiting examples of Wnt pathway agonists include GSK-3β inhibitors (e.g., CHIR99201, (6-[[2-[[4-(2,4-dichlorophenyl)-5-(5-methyl-1H-imidazol-2-yl)-2-pyrimidinyl]amino]ethyl]amino]-3-pyridinecarbonitrile)), 2-amino-4-[3,4-(methylenedioxy)benzyl-amino]-6-(3-methoxyphenyl)pyrimidine, TCS2002, TWS119, SB-216763, BIO ((2′Z,3′E)-6-Bromoindirubin-3′-oxime), lithium chloride, 5-(Furan-2-yl)-N-(3-(1H-imidazol-1-yl)propyl)-1,2-oxazole-3-carboxamide, lithium carbonate, CHIR98014 (6-N-[2-[[4-(2,4-Dichlorophenyl)-5-imidazol-1-ylpyrimidin-2-yl]amino]ethyl]-3-nitropyridine-2,6-diamine dihydrochloride), and SKL2001.

In some embodiments, the dosage range useful for a Wnt agonist (e.g., CHIR99021) is between 0.1 and 10 μM, for example, between 0.1 and 9 μM, between 0.1 and 8 μM, between 0.1 and 7 μM, between 0.1 and 6 μM, between 0.1 and 5 μM, between 0.1 and 4 μM, between 0.1 and 3 μM, between 0.1 and 2 μM, between 0.1 and 1 μM, between 1.5 and 10 μM, between 2 and 6 μM, between 4 and 6 μM, between 4.5 and 6.5 μM, between 1 and 9 μM, between 2 and 9 μM, between 3 and 9 μM, between 4 and 9 μM, between 5 and 9 μM, between 6 and 9 μM, between 7 and 9 μM, between 8 and 9 μM, or any integer therebetween.

In some embodiments, the dose of a Wnt agonist (e.g., CHIR99021) is e.g., at least 0.1 μM, at least 0.5 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, at least 2.5 μM, at least 3 μM, at least 4 μM, at least 5 μM, at least 6 μM, at least 7 μM, at least 8 μM, at least 9 μM, at least 10 μM, or more.

### Enriching the Culture for a Population of Induced Neural Crest Stem Cells

Neural crest cells are a group of cells that arise from the embryonic ectoderm germ layers and give rise to a diverse cell lineage. During development, extracellular signaling molecules, secreted from the adjacent epidermis and underlying mesoderm, such as Wnts, BMPs and FGFs, separate the non-neural ectoderm (epidermis) from the neural plate during neural induction. The neural crest lineage forms from intermediate levels of BMP signaling required for the development of the neural plate (low BMP) and epidermis (high BMP). A group of genes including, but not limited to Slug/Snail, FoxD3, Sox10, Sox9, AP-2 and c-Myc are activated in emergent neural crest cells. As referred to herein, neural crest stem cells are characterized by expression of nerve growth factor receptor p75^{NTR}. Neural crest stem cells are also marked by expression of AP-2α, Nestin, and PAX3.

Enriching/selecting for neural crest stem cells: The differentiation of odontoblasts from iPS cells as described herein involves, at the appropriate point, enrichment of the cell population for neural crest stem cells. An induced neural crest cell can be identified through the expression of markers important for neural crest cell development (e.g., p75^{NTR}, PAX3, SOX10). Thus, in some embodiments, cell cultures can be enriched for induced neural crest cells by selecting for cells that express markers for p75, PAX3, and/or SOX10. In the context of enriching a culture, the selection of cells that express markers for induced neural crest cells, the selection can physically sort or separate target from non-target cells. As used herein, the term “enriching” or “enrichment” refer to a process whereby the proportion of cells expressing a given phenotype or marker are selected to thereby increase their proportion in the population. The increase can be any increase as that term is defined herein, but will preferably be to a point where neural crest stem cells, defined at least by expression of p75^{NTR}, comprise at least 50%, 60%, 70%, 80%, 90%, 95% or more of the population. “Selecting” or “selection” in this context can refer to a positive or negative selection. A positive selection isolates, from a cell population, those cells that do express a given marker. A negative selection targets cells that do not express the given marker for elimination or removal. In one embodiment, the proportion of neural crest stem cells is enriched using magnetic beads (e.g., DYNABEADS™, ThermoFisher Scientific, Waltham, MA) bearing a ligand for a neural crest stem cell marker, e.g., p75NTR, to positively select those cells that express the marker. The enrichment thus physically selects and isolates cells that do express p75^{NTR} away from cells that do not. Other approaches, such as fluorescence activated cell sorting using a fluorescently labeled p75^{NTR} ligand, or selection on a surface, e.g., a microtiter dish surface coated with such a ligand are also specifically contemplated.

Where magnetic beads are to be used for enrichment or selection, beads can be attached to a ligand, e.g., an antibody, that specifically binds the desired marker. Where p75NTR is a cell-surface protein expressed on neural crest stem cells, it is well-suited for selection of cells. Anti p75NTR antibodies are commercially available, and include recombinant anti-p75 NGF receptor antibody [EP1039Y], catalog number ab52987, Abcam, Cambridge, UK; NGFR p75 antibody, catalog number sc-271708, Santa Cruz Biotechnologies, Santa Cruz, CA. Antibodies for PAX3 include recombinant anti-PAX3 antibody [HL160], catalog number ab308330, Abcam, Cambridge, UK; Pax3-7 antibody, catalog number sc-365843, Santa Cruz Biotechnologies, Santa Cruz, CA. Antibodies for SOX10 include recombinant anti-SOX10 antibody [SP267], catalog number ab227680, Abcam, Cambridge, UK; Sox10 antibody, catalog number sc-365692, Santa Cruz Biotechnologies, Santa Cruz, CA.

Where magnetic beads are to be used for enrichment or selection, beads can be attached to a ligand, e.g., an antibody, that specifically binds the desired marker. Where p75NTR is a cell-surface protein expressed on neural crest stem cells, it is well-suited for selection of cells. Anti p75NTR antibodies are commercially available, and include recombinant anti-p75 NGF receptor antibody [EP1039Y], catalog number ab52987, Abcam, Cambridge, UK; NGFR p75 antibody, catalog number sc-271708, Santa Cruz Biotechnologies, Santa Cruz, CA. Antibodies for PAX3 include recombinant anti-PAX3 antibody [HL160], catalog number ab308330, Abcam, Cambridge, UK; Pax3-7 antibody, catalog number sc-365843, Santa Cruz Biotechnologies, Santa Cruz, CA. Antibodies for SOX10 include recombinant anti-SOX10 antibody [SP267], catalog number ab227680, Abcam, Cambridge, UK; Sox10 antibody, catalog number sc-365692, Santa Cruz Biotechnologies, Santa Cruz, CA.

BMP agonists: Activators or agonists of Bone Morphogenic Protein (BMP) signaling are used, in combination with other factors as described herein, to promote differentiation of neural crest stem cells to odontoblasts. As used herein, the term “BMP pathway agonist” refers to an agent that activates the bone morphogenetic protein (BMP) pathway or inhibits the activity and/or expression of inhibitors of BMP signaling. A BMP pathway agonist as used herein can enhance signaling through the BMP pathway at any point along the pathway, for example, but not limited to increasing the expression and/or activity of BMP receptor, or BMP dependent genes and/or proteins, and decreasing the expression and/or activity of endogenous inhibitors of BMP receptor or decreasing the expression and/or activity of endogenous inhibitors of components of the BMP pathway, for example decreasing the expression of follistatin. Some non-limiting examples of BMP pathway agonists include BMP, e.g., recombinant BMP, e.g., recombinant BMP4 or BMP10, benzoxazole compounds, and ventromorphins. Additional description of the BMP pathway and its involvement with neural crest development can be found in Manzari-Tavakoli A, Babajani A, Farjoo M H, Hajinasrollah M, Bahrami S, Niknejad H. The Cross-Talks Among Bone Morphogenetic Protein (BMP) Signaling and Other Prominent Pathways Involved in Neural Differentiation. Front Mol Neurosci. 2022 Mar. 15; 15:827275. doi: 10.3389/fnmol.2022.827275. PMID: 35370542; PMCID: PMC8965007, which is incorporated by reference herein in its entirety.

FGF pathway agonists: Activators or agonists of Fibroblast Growth Factor (FGF) pathway signaling are used, in combination with other factors as described herein, to promote differentiation of neural crest stem cells to odontoblasts. As used herein, the term “FGF pathway agonist” or “FGF agonist” refers to an agent that activates the fibroblast growth factor (FGF) pathway or inhibits the activity and/or expression of inhibitors of FGF signaling, for example antagonists or inhibitors of FGF activity. A FGF activating agent as used herein can enhance signaling through the FGF pathway at any point along the pathway, for example, but limited to increasing the expression and/or activity of FGF receptor, or FGF dependent genes and/or proteins, and decreasing the expression and/or activity of endogenous inhibitors of FGF receptor or decreasing the expression and/or activity of endogenous inhibitors of components of the FGF pathway, for example decreasing the expression of protein kinase C (PKC). Some non-limiting examples of FGF pathway agonists include FGF (e.g., recombinant FGF), basic FGF (bFGF, e.g., recombinant bFGF), FGF8b (e.g., recombinant FGF8b), phosphatidylinositol-4,5-bisphosphate 3-kinase (PI3K)-AKT, phospholipase C gamma (PLCγ), and signal transducers and activators of transcription (STAT). The recombinant FGF is commercially available (Recombinant Human FGF-basic (bFGF), Peprotech, Catalog No. 100-188, Canbury, NJ; Human FGF-basic (FGF-2/bFGF)(aa-1-155) Recombinant Protein, ThermoFisher Scientific, Catalog No. PHG0266, Rochester, NY; Human FGF-basic (FGF-2/bFGF)(aa10-155) Recombinant Protein, ThermoFisher Scientific, Catalog No. PHG0021, Rochester, NY). Additional description of the FGF pathway and its involvement with neural crest development can be found in Manzari-Tavakoli A, Babajani A, Farjoo M H, Hajinasrollah M, Bahrami S, Niknejad H. The Cross-Talks Among Bone Morphogenetic Protein (BMP) Signaling and Other Prominent Pathways Involved in Neural Differentiation. Front Mol Neurosci. 2022 Mar. 15; 15:827275. doi: 10.3389/fnmol.2022.827275. PMID: 35370542; PMCID: PMC8965007, which is incorporated by reference herein in its entirety.

In some embodiments, the dosage range useful for fibroblast growth factor is between 10 and 800 pM, for example between 10 and 700 pM, between 10 and 600 pM, between 10 and 500 pM, between 10 and 400 pM, between 10 and 300 pM, between 10 and 200 pM, between 10 and 100 pM, between 100 and 800 pM, between 200 and 800 pM, between 300 and 800 pM, between 400 and 800 pM, between 500 and 800 pM, between 600 and 800 pM, between 700 and 800 pM, between 400 and 600 pM, between 450 and 650 pM, between 500 and 600 pM, between 400 and 500 pM, or any dose therebetween.

In one embodiment, the FGF pathway agonist can be an FGF receptor minibinder. As used herein, mini-protein binders, mini binders, or simply minibinders (mb) are computer designed proteins which bind to specific sequences of proteins in order to alter their function. Generally, a mini-protein binder is a geometrically tunable cyclic oligomer that can utilize idealized repeat domains in order to assist in the clustering of receptors and the magnification of activating or repressing signaling pathways. In various embodiments, repeat domains can comprise four identical repeats of a two helix module. Additional information regarding the structure and function of mini-protein binders can be found in Edman N I, Redler R L, Phal A, Schlichthaerle T, Srivatsan S R, Etemadi A, An S, Favor A, Ehnes D, Li Z, Praetorius F, Gordon M, Yang W, Coventry B, Hicks D R, Cao L, Bethel N, Heine P, Murray A N, Gerben S, Carter L, Miranda M, Negahdari B, Lee S, Trapnell C, Stewart L, Ekiert D C, Schlessinger J, Shendure J, Bhabha G, Ruohola-Baker H, Baker D. Modulation of FGF pathway signaling and vascular differentiation using designed oligomeric assemblies. bioRxiv [Preprint]. 2023 March 15:2023.03.14.532666. doi: 10.1101/2023.03.14.532666. PMID: 36993355; PMCID: PMC10055045.

In some embodiments, the mini-protein binder is directed toward FGFR (FGFR2 mini-binder, FGFR_mb). In some embodiments, the FGFR mini-protein binder contains the binding sequence MGDRRKEMDKVYRTAYKRITSTPDKEKRKEVVKEATEQLRRIAKDEEEKKKAAYMISFLKT LGLEHHHHIH (SEQ ID NO: 7). The mini-protein binder can contain a detection tag on either the N- or C-terminus of the binding sequence. In some embodiments, the detection tag is MSHHHHHHHHSENLYFQSGGG (SEQ ID NO: 8).

In some embodiments, the minibinder has a sequence at least 85%, at least 87%, at least 90%, at least 91%, at least 92%, at least 93%, at least 94%, at least 95%, at least 96%, at least 97%, at least 98%, at least 99%, or more, identical to a native or reference sequence of the mini-protein binder and retains at least 50% of the binding activity of the reference minibinder polypeptide. Binding affinity can be assayed/confirmed as described, for example, in Edman N I, Redler R L, Phal A, Schlichthaerle T, Srivatsan S R, Etemadi A, An S, Favor A, Ehnes D, Li Z, Praetorius F, Gordon M, Yang W, Coventry B, Hicks D R, Cao L, Bethel N, Heine P, Murray A N, Gerben S, Carter L, Miranda M, Negahdari B, Lee S, Trapnell C, Stewart L, Ekiert D C, Schlessinger J, Shendure J, Bhabha G, Ruohola-Baker H, Baker D. Modulation of FGF pathway signaling and vascular differentiation using designed oligomeric assemblies. bioRxiv [Preprint]. 2023 March 15:2023.03.14.532666. doi: 10.1101/2023.03.14.532666. PMID: 36993355; PMCID: PMC10055045. The degree of homology (percent identity) between a reference and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Techniques for designing and using mini-protein binders are established and include, for example, those disclosed by Cao et al. (2022, ‘Design of protein-binding proteins from the target structure alone’, Nature, 605: 551-60), and International Application No. PCT/US22/73590, which are herein incorporated by reference in their entireties.

Sonic Hedgehog signaling pathway activators: In the methods described herein, one or more Hedgehog (Hh) signaling pathway modulating agents can be used in the induction of differentiation of an iPS cell to an odontoblast. Hh proteins are key molecules for diverse tissue patterning processes in both invertebrates and vertebrates. For example, in Drosophila, Hh is crucial for the development of a segmented body plan and the patterning of imaginal tissues, whereas in vertebrates Sonic hedgehog (Shh) functions to pattern limb buds and promote cell fate specification, proliferation, and axon guidance in the central nervous system. Shh initiates signaling by binding the 12-pass transmembrane protein Patched1 (PTCH1). Upon Shh binding, the inhibition exerted by PTCH1 on the transmembrane protein Smoothened (Smo) is relieved, eliciting a signaling cascade which ultimately leads to Gli-mediated transcription.

As used herein, the term “hedgehog pathway agonist” refers to any agent that activates the hedgehog pathway or inhibits the activity and/or expression of inhibitors of hedgehog signaling, for example antagonists or inhibitors of hedgehog activity. A hedgehog activating agent as used herein can enhance signaling through the hedgehog pathway at any point along the pathway, for example, but limited to increasing the expression and/or activity of hedgehog receptor, or hedgehog dependent genes and/or proteins, and decreasing the expression and/or activity of endogenous inhibitors of hedgehog receptor or decreasing the expression and/or activity of endogenous inhibitors of components of the hedgehog pathway, for example decreasing the expression of Gli truncated. Agents that induce the SHH pathway include, but are not limited to, smoothened agonist (e.g., SAG, purmorphamine), inhibitors of Patched-1 (e.g., cyclopamine) or SHH ligands.

In some embodiments, the dosage range for SAG used, in part, to promote differentiation of iPSC to odontoblasts is between 200 nM and 10 μM, for example, between 200 nM and 1 μM, between 200 nM and 750 nM, between 200 nM and 500 nM, between 200 nM and 400 nM, between 200 nM and 300 nM, between 300 nM and 1 μM, between 400 nM and 1 μM, between 500 nM and 1 μM, between 500 nM and 1 μM, between 600 nM and 1 μM, between 700 nM and 1 μM, between 800 nM and 1 μM, between 900 nM and 1 μM, between 300 nM and 600 nM, between 200 nM and 500 nM, between 300 nM and 500 nM, between 350 nM and 450 nM, or any range therebetween.

### Cell Culture

As used herein, a basal neural maintenance medium is used to help induced pluripotent stem cells differentiate into induced neural crest stem cells.

Basal neural maintenance medium (BNMM) is a medium, supplemented with factors as described herein, used to promote differentiation of iPSCs to induced neural crest cells. BNMM is comprised of Dulbecco's Modified Eagle Medium F12+glutamine: neurobasal medium (1:1), wherein the neurobasal medium comprises N2 supplement, B27, Glutamax, ITS-A, β-mercaptoethanol, and non-essential amino acids (NEAA). BNMM can be in contact with iPSCs for at least one day, at least two days, at least three days, at least four days, at least five days, at least six days, at least seven days, at least eight days, at least nine days, at least ten days, at least eleven days. As noted, BNMM can also contain other agents, such as TGF-β/SMAD inhibitor(s) and Wnt activators used to promote differentiation of iPS cells to neural crest stem cells. Exemplary timelines for the treatment or contacting with the various factors are provided in FIG. 5 herein. In some embodiments, the SMAD inhibitors can be SB4315442 and/or LDN193189, and the Wnt activator can be CHIR99021. The SB4315442 can be in the BNMM medium contacted with the cells for at least zero days, at least one day, at least two days, at least three days, or at least four days. The LDN 193189 can be in the BNMM medium contacted with the cells for at least one day, at least two days, or at least three days. The CHIR99021 can be in the BNMM medium for at least one day, at least two days, at least three days, at least four days, at least five days, at least six days, at least seven days, at least eight days, at least nine days, at least ten days. The CHIR99021 can be in the BNMM medium two days after the iPSCs have come in contact with the BNMM medium. The BNMM medium can be removed from the iPSCs and new BNMM medium can be replaced. In some embodiments, the BNMM medium is replaced daily.

In some embodiments, the media can also include L-alanyl-L-gultamine dipeptide in 0.85% NaCl (GLUTAMAX™). The terms “L-alanyl-L-glutamine dipeptide in 0.85% NaCl”, “L-alanyl-L-glutamine” and “GLUTAMAX” can be used interchangeably throughout the application. GLUTAMAX can be found commercially at ThermoFisher Scientific, catalog number 35050061, Waltham, MA. In some embodiments, GLUTAMAX can be added to the media to at least 100 nM, at least 150 nM, at least 200 nM, at least 250 nM, at least 300 nM, at least 350 nM, at least 400 nM, at least 450 nM, at least 500 nM or more.

N-2 Supplement is a chemically defined, serum-free supplement for growing or maintaining neuronal cells based on Bottenstein's N-1 formulation (Bottenstein, J. E. (1985) Cell Culture in the Neurosciences, Plenum Press: New York and London). N-2 supplement can be found commercially at ThermoFisher Scientific, catalog number 17502048, Waltham, MA. In some embodiments, N-2 supplement can be added to the media at least 100 nM, at least 150 nM, at least 200 nM, at least 250 nM, at least 300 nM, at least 350 nM, at least 400 nM, at least 450 nM, at least 500 nM or more.

B-27 supplement is a defined yet complex mixture of antioxidant enzymes, proteins, vitamins, and fatty acids that are combined in optimized ratios to support neuronal survival in culture. The original serum-free neuronal culture supplement formula developed by Dr. Gregory Brewer and colleagues is described in Brewer et al., J Neuroscience Res 35: 567-576, 1993 and Brewer and Cotman, Brain Res 494: 65-74, 1989 [1,2].

Non-Essential Amino Acids are used as a supplement for cell culture medium, to increase cell growth and viability. The formulation of Non-Essential Amino Acids includes glycine (10 mM), L-Alanine (10 mM), L-Asparagine (10 mM), L-Aspartic acid (10 mM), L-glutamic acid (10 mM), L-proline (10 mM), and L-serine (10 mM). Non-Essential Amino Acids can be found commercially at ThermoFisher Scientific, catalog number 11140050, Waltham MA. In some embodiments, Non-Essential Amino Acids can be added to the media at least 100 nM, at least 150 nM, at least 200 nM, at least 250 nM, at least 300 nM, at least 350 nM, at least 400 nM, at least 450 nM, at least 500 nM or more.

As used herein, odontoblast medium is a medium used with agents or factors as described herein to differentiate induced neural crest cells as described herein to differentiate to odontoblasts. The odontoblast medium can contain Dulbecco's Modified Eagle Medium+Glutamax, dexamethasone, fetal bovine serum, β-glycerophosphate, and L-ascorbic acid. Odontoblast medium can be in contact with induced neural crest cells for at least one day, at least two days, at least three days, at least four days, at least five days, at least six days, at least seven days, at least eight days, at least nine days, at least ten days, at least eleven days, at least twelve days, at least thirteen days, at least fourteen days, at least fifteen days, or at least sixteen days. Odontoblast medium can be changed, replacing with fresh odontoblast medium with appropriate differentiation factors at appropriate intervals during the differentiation protocol. Odontoblast medium will contain or be supplemented at appropriate times with differentiation factors including a BMP pathway agonist, e.g., BMP4, a FGFR agonist, e.g., FGF or an FGF superagonist, and a Hedgehog agonist, e.g., SAG. The agonists can be at different concentrations. Exemplary timelines for the treatment or contacting with the various factors are provided in FIG. 5 herein. The agonists can be the same concentrations. The BMP4 can be in the medium for at least one day after enrichment, at least two days after enrichment, at least three days after enrichment, at least four days after enrichment, at least five days after enrichment, at least six days after enrichment, at least seven days after enrichment, at least eight days after enrichment, at least nine days after enrichment, at least ten days after enrichment, at least eleven days after enrichment, at least twelve days after enrichment, at least thirteen days after enrichment, at least fourteen days after enrichment, at least fifteen days after enrichment, or at least sixteen days after enrichment. The FGFR superagonist can be in the medium for at least at least one day after enrichment, at least two days after enrichment, at least three days after enrichment, at least four days after enrichment, at least five days after enrichment, at least six days after enrichment, at least seven days after enrichment, or at least eight days after enrichment. The SAG can be in the medium for at least at least one day after enrichment, at least two days after enrichment, at least three days after enrichment, at least four days after enrichment, at least five days after enrichment, at least six days after enrichment, at least seven days after enrichment, or at least eight days after enrichment.

mTeSR1 stem cell medium is a medium used to grow iPSCs to confluence. mTeSR1 stem cell medium is commercially available (StemCell Technologies, catalog number #85850, Vancouver, CA). The mTeSR1 stem cell medium can be in contact with iPSCs for at least one day, at least two days, at least three days, at least four days, at least five days, at least six days, at least seven days, at least eight days, at least nine days, at least ten days, at least eleven days, at least twelve days, at least thirteen days, at least fourteen days or more. mTeSR1 stem cell medium can also contain biologics that modulate or maintain phenotype. In some embodiments, the biologics can be insulin, bFGF, TGFβ, and ROCK inhibitor. The biologics can be at different concentrations. The biologics can be at the same concentrations. The mTeSR1 stem cell medium can be removed and replaced daily until the iPSCs reach at least 70% confluency.

The odontoblasts or the cells from which they are produced can be cultured in a conventional two-dimensional culture initially, where they grow primarily in monolayers, and then transferred to a 3D culture, or they may be placed directly into 3D culture. Odontoblasts can self-assemble into organoids when plated on ultra-low attachment plates (CORNING ELPLASIA plates, Catalog No. 4441, Corning Life Sciences, Big Flats, NY). When 3D growth is desired, a cell or cell type, including odontoblasts prepared as described herein, can be mixed with a substrate such as a basement membrane matrix (e.g., BD Matrigel™ from BD Bioscience, San Jose Calif; catalog #356234; see Hughes et al., Proteomics 10(9):1886-1890, 2010), and grown in vitro using growth media (e.g., DMEM-high sucrose). Exemplary materials for 3D culture growth include, but are not limited to: BD Matrigel™ Basement Membrane Matrix, and growth-factor reduced Matrigel™. While the precise composition of the matrix can vary, it can include a mixture of extracellular matrix proteins and growth factors, and it can be derived from tumor cells (e.g., Engelbreth-Holm-Swarm (EHS) mouse sarcoma cells). The matrix can be one that, in the absence of other factors, maintains a pluripotent cell in its pluripotent state (i.e., it promotes self-renewal).

In three-dimensional cultures, the cells can become multilayered or clustered with the layered and/or clustered cell growth being guided by scaffolds. For example, 3D cultures can be grown in AggreWell™ 400 plates (Stemcel Technologies, Grenoble, France). The 3D cultures of odontoblasts or organoids can be grown in bioreactors to support spheroid growth and the development of an extracellular matrix. More generally, one can use 3D cultures based on or generated by extracellular matrices or scaffolds (e.g., employing hydrogels), modified surfaces, rotating bioreactors, microcarriers, magnetic levitation, hanging drop plates, and/or magnetic 3D bioprinting. In scaffold-free techniques, one can employ low adhesion plates and micropatterned surfaces.

Exemplary extracellular matrices include, but are not limited to Matrigel™ (e.g., a basement-membrane matrix extracted from Engelbreth-Holm-Swarm mouse sarcomas), matrices from other cell types and synthetic matrices (see eg., Aisenbrey, E. A., Murphy, W. L. Synthetic alternatives to Matrigel. Nat Rev Mater 5, 539-551 (2020) which is incorporated herein by reference in its entirety).

### Scaffold and Matrix Material

Various embodiments of the compositions and methods described herein employ a scaffold seeded with progenitor cells, epithelial cells, mesenchymal cells, odontoblast precursors, odontoblasts, a combination thereof or an organoid comprising odontoblasts. In some embodiments, the odontoblasts or odontoblast organoids are grown in or on a scaffold in the presence of a mineralizing solution. Accordingly, mineralized materials can be produced in or on the scaffold. The shape and characteristics of the scaffold can be chosen so as to provide a desired framework for generating a 3D odontoblast organoid or in a shape that can be mineralized and implanted into a subject.

A scaffold can be fabricated with any matrix material recognized as useful by the skilled artisan. A matrix material can be a biocompatible material that generally forms a porous, microcellular scaffold, which provides a physical support for cells migrating thereto. Such matrix materials can: allow cell attachment and migration; deliver and retain cells and biochemical factors; enable diffusion of cell nutrients and expressed products; or exert certain mechanical and biological influences to modify the behavior of the cell phase. The matrix material generally forms a porous, microcellular scaffold of a biocompatible material that provides a physical support and an adhesive substrate for growth of cells during in vitro or in vivo culturing.

The matrix comprising the scaffold can have an adequate porosity and an adequate pore size so as to facilitate cell growth and diffusion throughout the whole structure of both cells and nutrients. In embodiments where the scaffold is implanted, the matrix can be biodegradable providing for absorption of the matrix by the surrounding tissues (if implanted), which can eliminate the necessity of a surgical removal. The rate at which degradation occurs can coincide as much as possible with the rate of tissue or organ formation. Thus, while cells are fabricating their own natural structure around themselves (e.g., dentin, cementum, enamel), the matrix is able to provide structural integrity and eventually break down, leaving the neotissue, newly formed tissue or organ which can assume the mechanical load. The matrix can be an injectable matrix in some configurations. The matrix can be delivered to a tissue using minimally invasive endoscopic procedures.

The scaffold can comprise a matrix material having different phases of viscosity. For example, a matrix can have a substantially liquid phase or a substantially gelled phase. The transition between phases can be stimulated by a variety of factors including, but not limited to, light, chemical, magnetic, electrical, and mechanical stimulus. For example, the matrix can be a thermosensitive matrix with a substantially liquid phase at about room temperature and a substantially gelled phase at about body temperature. The liquid phase of the matrix can have a lower viscosity that provides for optimal distribution of growth factors or other additives and injectability, while the solid phase of the matrix can have an elevated viscosity that provides for matrix retention at or within the target tissue.

The scaffold can comprise a matrix material formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above. Alternatively, the matrix can be formed of naturally occurring biopolymers. Such naturally occurring biopolymers include, but are not limited to, fibrin, fibrinogen, fibronectin, collagen, and other suitable biopolymers. Also, the matrix can be formed from a mixture of naturally occurring biopolymers and synthetic polymers.

The scaffold can include one or more matrix materials including, but not limited to, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, polyacrylate, or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, or nylon.

The scaffold can further comprise any other bioactive molecule, for example an antibiotic or an additional chemotactic growth factor or another osteogenic, dentinogenic, amelogenic, odontogenic, or cementogenic growth factor. In some embodiments, the scaffold is strengthened, through the addition of, e.g., human serum albumin (HSA), hydroxyethyl starch, dextran, or combinations thereof. Suitable concentrations of these compounds for use in the compositions of the application are known to those of skill in the art, or can be readily ascertained without undue experimentation. The concentration of compound in the scaffold will vary with the nature of the compound, its physiological role, and desired therapeutic or diagnostic effect. A therapeutically effective amount is generally a sufficient concentration of therapeutic agent to display the desired effect without undue toxicity. The compound can be incorporated into the scaffold or matrix material by any known method. In some embodiments, the compound is imbedded in a gel, e.g., a collagen gel incorporated into the pores of the scaffold or matrix material.

Alternatively, chemical modification methods can be used to covalently link the compound to a matrix material. The surface functional groups of the matrix can be coupled with reactive functional groups of the compound to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Pores and channels of the scaffold can be engineered to be of various diameters. For example, the pores of the scaffold can have a diameter range from micrometers to millimeters. In some embodiments, the pores of the matrix material include microchannels. Microchannels generally have an average diameter of about 0.1 μm to about 1,000 μm, e.g., about 50 μm to about 500 μm (for example about 100 μm, 150 μm, about 200 μm, about 250 μm, about 300 μm, about 350 μm, about 400 μm, about 450 μm, about 500 μm, or about 550 m). One skilled in the art will understand that the distribution of microchannel diameters can have any distribution including a normal distribution or a non-normal distribution. In some embodiments, microchannels are a naturally occurring feature of the matrix material(s). In other embodiments, microchannels are engineered to occur in the matrix materials.

Several methods can be used for fabrication of porous scaffolds, including particulate leaching, gas foaming, electrospinning, freeze drying, foaming of ceramic from slurry, and the formation of polymeric sponge. Other methods that can be used for fabrication of porous scaffolds include computer aided design (CAD) and synthesizing the scaffold with a bioplotter (e.g., solid freeform fabrication) (e.g., Bioplotter™, EnvisionTec, Germany).

Biologic drugs that can be added to the scaffold or matrix compositions include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner that enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions described herein can also be modified to incorporate a diagnostic agent, such as a radiopaque agent. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents that can be utilized in the compositions of the invention can be readily ascertained by those of skill in the art and can include, for example, the use of radiolabeled fatty acids or analogs thereof.

The concentration of an agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of a tissue graft, while minimizing potential toxicity. In any event, the desired concentration in a particular instance for a particular compound is readily ascertainable by one of skill in the art.

### Detection of Odontoblasts and Precursors Thereof

The production of odontoblasts can be confirmed by detecting one or more markers of odontoblasts or by detecting the presence of functions associated with odontoblasts, such as dentin production. In some embodiments, the production of odontoblasts can be confirmed by detecting the presence of one or more markers of odontoblast differentiation including, but not limited to DSPP, and RUNX2. In addition to DSPP, odontoblasts also express Nestin, secrete the extracellular matrix protein, reelin (RELN), and express the transcription factor RUNX2. Antibodies for the detection of these markers are commercially available and can be found at (anti-nestin, Catalog number: sc-23927, Santa Cruz Biotechnologies, Santa Cruz, CA; anti-DSPP, Catalog number: sc-73632, Santa Cruz Biotechnologies, Santa Cruz, CA; anti-RUNX2, Catalog number: sc-390351, Santa Cruz Biotechnologies, Santa Cruz, CA; Recombinant Anti-Reelin antibody [EPR26278-30], Catalog number: ab312310, Cambridge, UK).

Alternatively, one can detect the presence of odontoblasts by detecting dentin production or deposition of calcium when cells are cultured in the presence of a mineralizing solution (e.g., calcium 2.5 mM, phosphate 1.5 mM). Alizarin Red staining can be used to evaluate mineralization as described in the Examples herein.

In some embodiments, cells in the oral epithelium stage can be detected by a relative increase in gene expression of DSPP, NESTIN, and RUNX2 as markers, e.g., by at least 10% compared to cells in the prior stage (e.g., induced neural crest stem cells). In some embodiments, the levels of DSPP, NESTIN and RUNX2 are increased by at least 20%, at least 30%, at least 40% at least 50%, at least 60%, at least 70%, at least 80%, at least 90%, at least 1-fold, at least 2-fold, at least 5-fold, at least 10-fold, at least 100 fold or more at the sub-odontoblast stage compared to an earlier stage in the method (e.g., induced neural crest stem cells).

### Mineralization

Dental mineralization occurs within the odontoblast, and such mineralization can provide benefits, whether for the in vitro production of dentin or dentin-related materials useful, e.g., for dental repair, or for in vivo, cell-mediated dental repair. Dental or dental-related mineralization can be measured, for example, using Alizarin red staining. Alizarin red staining is a staining technique to locate calcium deposits in tissues. One of ordinary skill in the art will be able to use Alizarin red staining to measure the amount of calcium deposits in a given tissue (e.g., odontoblast-containing tissue).

Dentin is a calcified tissue of the body and is one of the four major components of teeth.

There are three different types of dentin, referred to as primary dentin, secondary dentin, and tertiary dentin. Tertiary dentin is more dense than other forms of dentin and is only formed by an odontoblast directly affected by a stimulus; therefore, the architecture and structure depend on the intensity and duration of the stimulus. Tertiary dentin is also known as osteodentin.

### Pharmaceutically Acceptable Carriers

In one aspect, the methods of introducing or replacing dentin or enamel-producing cells in a subject as described herein involve the use of therapeutic compositions comprising odontoblasts or organoids comprising odontoblasts. Therapeutic compositions can contain a physiologically tolerable carrier together with the cell composition and optionally at least one additional bioactive agent as described herein, dissolved or dispersed therein as an active ingredient. Alternatively, a therapeutic composition can contain dentin produced using odontoblasts as described herein, or dentin and enamel produced using odontoblasts and ameloblasts separately or in coculture. In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as implantable either as liquid solutions or suspensions, however, solid forms suitable for solution, or suspensions, in liquid prior to use can also be prepared. Formulation in a gel, scaffold, paste or amalgam for implantation is also specifically contemplated.

In general, the human odontoblasts or organoids thereof described herein can be administered as a suspension or admixture with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the human odontoblasts as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Administration and Efficacy

Provided herein are methods for treating a disease or disorder comprising dentin and/or enamel problems (e.g., demineralization or impaired dentin or enamel production during development) by administering human odontoblasts, organoids comprising odontoblasts, or a dental repair composition comprising odontoblasts, organoids comprising odontoblasts, or dentin or enamel prepared as described herein to a subject in need thereof.

In some embodiments, the methods of treating a disease or disorder comprise first diagnosing a subject with a dental dentin or dentin/enamel problem that requires treatment. In other embodiments, the degree of dentin loss or impaired dentin or enamel production during development is first assessed using one or more measured or measurable parameters including clinically detectable markers of disease, for example, dentin defects or deficiency, enamel discoloration, tooth sensitivity to heat/cold, chips or cracks in enamel, scanning electron microscopy, stereo microscopy, white light 3D profilometry, and atomic force microscopy. It will be understood, however, that the dosage of administered odontoblasts, organoids thereof, or dentin produced by odontoblasts and total usage of the compositions and formulations as disclosed herein will be decided by the attending clinician within the scope of sound medical judgment. The exact amount required will vary depending on factors such as the type of disease being treated.

The term “effective amount” as used herein refers to the amount of a population of odontoblasts or organoids thereof needed to alleviate at least one or more symptom of impaired dental structure, and relates to a sufficient amount of a composition to provide the desired effect, e.g., treat a subject to augment or replace dentin or other dental structure. The term “therapeutically effective amount” therefore refers to an amount of, e.g., human odontoblasts or an organoid comprising odontoblasts that is sufficient to promote a particular effect when administered to a typical subject, such as one who has or is at risk for dentin deficiency, loss or impaired dentin production. An effective amount as used herein would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting dental structure prior to administering the cells or other compositions as described herein according to the methods described herein. In some embodiments, the subject is first diagnosed as being at risk of developing a disease or disorder comprising dentin loss or impaired dentin production prior to administering the cells or other compositions as described herein.

For use in the various aspects described herein, an effective amount of human odontoblast cells can comprise at least 10² odontoblast cells, at least 5×10² odontoblast cells, at least 10³ odontoblast cells, at least 5×10³ odontoblast cells, at least 10⁴ odontoblast cells, at least 5×10⁴ odontoblast cells, at least 10⁵ odontoblast cells, at least 2×10⁵ odontoblast cells, at least 3×10⁵ odontoblast cells, at least 4×10⁵ odontoblast cells, at least 5×10⁵ odontoblast cells, at least 6×10⁵ odontoblast cells, at least 7×10⁵ odontoblast cells, at least 8×10⁵ odontoblast cells, at least 9×10⁵ odontoblast cells, at least 1×10⁶ odontoblast cells, at least 2×10⁶ odontoblast cells, at least 3×10⁶ odontoblast cells, at least 4×10⁶ odontoblast cells, at least 5×10⁶ odontoblast cells, at least 6×10⁶ odontoblast cells, at least 7×10⁶ odontoblast cells, at least 8×10⁶ odontoblast cells, at least 9×10⁶ odontoblast cells, at least 10⁷ odontoblast cells, at least 2×10⁷ odontoblast cells, at least 3×10⁷ odontoblast cells, at least 4×10⁷ odontoblast cells, at least 5×10⁷ odontoblast cells, at least 6×10⁷ odontoblast cells, at least 7×10⁷ odontoblast cells, at least 8×10⁷ odontoblast cells, at least 9×10⁷ odontoblast cells, at least 1×10⁸ odontoblast cells, at least 2×10⁸ odontoblast cells, at least 3×10⁸ odontoblast cells, at least 4×10⁸ odontoblast cells, at least 5×10⁸ odontoblast cells, at least 6×10⁸ odontoblast cells, at least 7×10⁸ odontoblast cells, at least 8×10⁸ odontoblast cells, at least 9×10⁸ odontoblast cells, at least 1×10⁹ odontoblast cells, at least 2×10⁹ odontoblast cells, at least 3×10⁹ odontoblast cells, at least 4×10⁹ odontoblast cells, at least 5×10⁹ odontoblast cells, at least 6×10⁹ odontoblast cells, at least 7×10⁹ odontoblast cells, at least 8×10⁹ odontoblast cells, at least 9×10⁹ odontoblast cells, or more.

The odontoblast cells can be derived from one or more donors, or can be obtained from an autologous source. In some embodiments of the aspects described herein, the odontoblast cells are expanded in culture prior to administration to a subject in need thereof.

Exemplary modes of administration for use in the methods described herein include, but are not limited to, local administration (e.g., using a paste), injection and implantation (with or without a scaffold material).

In some embodiments of the aspects described herein, one or more routes of administration are used in a subject to achieve distinct effects. For example, odontoblasts can be administered to a subject by both implantation and local administration routes for treating or repairing tooth structure. In such embodiments, different effective amounts of the odontoblast cells can be used for each administration route.

In some embodiments, additional agents to aid in treatment of the subject can be administered before or following treatment with the odontoblast cells described herein. Such additional agents can be used, e.g., to prepare tooth or gum tissue for administration of the odontoblasts cells. Alternatively, the additional agents can be administered after the odontoblast cells to support the engraftment and growth of the administered cell in the tissue.

The efficacy of treatment can be determined by the skilled clinician (e.g., a dental assistant or dentist). However, a treatment is considered “effective treatment,” as the term is used herein, if any one or all of the symptoms, or other clinically accepted symptoms or markers of dental structure/dentin/mineralization deficiency are reduced, e.g., by at least 10% following treatment with a composition comprising or produced by human odontoblast cells as described herein. Methods of measuring these indicators are known to those of skill in the art and/or described herein.

Indicators of a dentin- or enamel-related disease, disorder, or injury include one or more of, e.g., pain, sensitivity to temperature, cracks, chips, caries, discoloration, or by clinical means such as scanning electron microscopy, stereo microscopy, white light 3D profilometry, and atomic force microscopy.

### Treatment of Dentin Disorders

The methods and compositions provided herein relate to the generation and use of human odontoblasts. Accordingly, provided herein are methods for the treatment and prevention of a disease or disorder associated with a dentin disorder in a subject in need thereof. The methods described herein can be used to treat, ameliorate, prevent or slow the progression of a number of dentin-related diseases or their symptoms, such as those resulting in damage or demineralization to dental structure.

The methods described herein can also be used to treat or ameliorate acute or chronic dentin issues or their symptoms or complications, including dentinogenesis imperfecta, dentin hypersensitivity, dentin dysplasia (type I and type II), Trich-dento-osseous, and dentinal sclerosis; amelogenesis imperfecta,

One type of disease or disorder affecting dental structure is Trich-dento-osseous (TDO) syndrome. TDO is a is a rare, autosomal dominant disorder principally characterized by curly hair at infancy, severe enamel hypomineralization and hypoplasia and taurodontism of teeth, sclerotic bone, and other defects.

Another type of disease, disorder, or injury that can be affected by odontoblasts and/or dentin is amelogenesis imperfecta. Amelogenesis imperfecta is a congenital disorder that results in a rare, abnormal formation of the enamel where ameloblastin, enamelin, tuftelin, and amelogenin are mutated and result of abnormal enamel formation via amelogenesis. People with amelogenesis imperfecta may have teeth with abnormal color: yellow, brown or grey; this disorder can affect any number of teeth of both dentitions. The teeth have a higher risk for dental cavities and are hypersensitive to temperature changes as well as rapid attrition, excessive calculus deposition, and gingival hyperplasia.

As used herein, the terms “administering,” “introducing” and “transplanting” are used interchangeably in the context of the placement of cells, e.g. odontoblasts or organoids comprising odontoblasts, as described herein into a subject, by a method or route which results in at least partial localization of the introduced cells at a desired site, such as a site of injury or repair, such that a desired effect(s) is produced. The cells e.g. odontoblasts, or their differentiated progeny can be implanted directly to the tooth, gums or the mouth, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment.

When provided prophylactically, odontoblast cells or organoids as described herein can be administered to a subject in advance of any symptom of dental structure loss or impaired dental development. Accordingly, the prophylactic administration of odontoblasts or organoids thereof serves to prevent dental structure loss or impaired dental production.

When provided therapeutically, odontoblasts are provided at (or after) the onset of a symptom or indication of dental structure disorder, e.g., upon the detection of one or more sites of dentin and/or enamel loss.

In some embodiments of the aspects described herein, the odontoblasts or organoids thereof being administered according to the methods described herein comprise allogeneic odontoblasts obtained from one or more donors. As used herein, “allogeneic” refers to an odontoblast derived from somatic cell derived-iPSCs obtained from one or more different donors of the same species, where the genes at one or more loci are not identical. For example, an odontoblast or organoid thereof being administered to a subject can be derived from cells obtained from one more unrelated donor subjects, or from one or more non-identical siblings. In some embodiments, syngeneic odontoblasts can be used, such as those obtained from genetically identical animals, or from identical twins. In other embodiments of this aspect, the odontoblasts are autologous cells; that is, the odontoblasts are differentiated from stem cells, e.g., iPS cells, derived from a subject and administered to the same subject, i.e., the donor and recipient are the same.

### Dental Repair Compositions

Provided herein, in some embodiments, are dental repair compositions for use in the treatment of dentin or dental structure disorders. The composition can comprise odontoblast cells produced as described herein, odontoblast cells plus ameloblast cells (e.g., in vitro-differentiated ameloblast cells, which are further described in PCT/US22/53517, which is incorporated by reference herein in its entirety) or products produced by such odontoblasts or odontoblast/ameloblast combinations. In one embodiment, such dental repair compositions comprise dentin produced by in vitro differentiated odontoblast cells. In other embodiments, the dental repair composition comprises enamel produced by in vitro-differentiated ameloblasts.

In some embodiments, the dental repair composition further comprises calcium phosphate or hydroxyapatite. Other components of such dental repair compositions can include one or more of amelogenin (AMELX), or enamelin (ENAM).

Dental repair compositions can be in the form of an implant (e.g., a tooth implant) or can be a dental composite for application to an existing tooth with cracked, chipped, demineralized or otherwise dysfunctional dental structure.

### Screening Platforms Using Stem Cell-Derived Odontoblasts

The matured odontoblasts or organoids thereof prepared as described herein provide a platform for the study or evaluation of the likely effects of known or experimental drugs that can impact dental structure production. In addition, the matured odontoblasts or organoids thereof prepared as described herein can be used to assess functional changes in response to genomic modifications or mutations. In particular, odontoblasts or organoids produced as described herein derived from a subject having a given disease can be used to model a given disease in a dish and to screen for agents or genomic modification that regulate disease. By screening with, for example, a library or collection of potential drugs or agents, odontoblasts or organoids thereof prepared and matured as described herein can also be used to identify new drugs with beneficial effects on odontoblast viability or dentin production.

Odontoblasts or organoids thereof derived from normal donor cells can provide useful information in both situations, and odontoblasts derived from donors with dental demineralization or other structural issues, or derived from cells engineered to mimic a dental disease or disorder can be very useful in identifying new drugs or agents to treat such diseases.

Screening assays can also be used in combination with mutagenesis assays to test for correction of disease in patient cell lines, odontoblasts derived from a patient having dentin dysfunction, or in odontoblast organoids prepared as described herein. Such mutations can be introduced using any known genome modification system including, but not limited to, base or prime genome editors, CRISPR/Cas, zinc finger nucleases (ZFN), transcription activator-like effector nucleases (TALENs), and the like and their effect on function can be assessed.

In either instances, the evaluation of functional or structural parameters as described herein or as known in the art can be informative with regard to the effects of a given agent or given mutation. In general, such assays comprise contacting odontoblasts prepared and matured as described herein with an agent and measuring one or more parameters of the odontoblasts described herein as an indicator of the agent's effect(s) or introducing a mutation to the genome of the odontoblast and measuring one or more functional parameters. Where effects are observed, dose responses can also be evaluated by varying the concentration of the agent and/or the duration of contacting.

Accordingly, stem cell-derived odontoblasts prepared and matured as described herein can be used to identify an agent, evaluate an agent, or evaluate a genomic modification for its effect on parameters such as expression of markers, cell viability, mineral deposition, dentin or enamel production or other parameters described herein or known in the art.

In some embodiments, odontoblasts or organoids comprising odontoblasts prepared as described herein can be used to identify targets for genetic manipulation including, but not limited to gene editing to modify a disease phenotype. As a non-limiting example, odontoblast-comprising organoids prepared as described herein using iPS cells derived from a subject with TDO can be subjected to a random mutagenesis or base-editing regimen and assayed for changes in expression of DSPP or other markers of dentin production. Clones exhibiting favorable changes in phenotype can be analyzed to determine the genetic change(s) involved in bringing about the phenotypic change, thereby identifying gene targets for therapeutic manipulation, including but not limited to targeted genomic modification.

In some embodiments, stem cell-derived odontoblasts can be used in assays to screen agents, selected from small molecules, nucleic acids or analogues thereof, aptamers; proteins or polypeptides or analogues or fragments thereof, among other agents for effects, detrimental or beneficial, on the cells.

In some embodiments, the agent is an agent of interest including known and unknown compounds that encompass numerous chemical classes, primarily organic molecules, which may include organometallic molecules, inorganic molecules, genetic sequences, etc. Candidate agents also include organic molecules comprising functional groups necessary for structural interactions, particularly hydrogen bonding, and typically include amine, carbonyl, hydroxyl or carboxyl groups, frequently more than one of such functional chemical groups. The candidate agents often comprise cyclic carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Compounds, including candidate agents, can be obtained from any of a variety of sources including libraries of synthetic or natural compounds. Various means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g., ground water, sea water, etc.; biological samples, e.g. lysates prepared from crops, tissue samples, etc.; manufacturing samples, e.g. time course during preparation of pharmaceuticals; as well as libraries of compounds prepared for analysis; and the like.

In some embodiments, the effect of the agent or the effect of a given genomic modification is determined by quantifiable parameters of stem cell-derived odontoblasts, such as expression of DSPP, mineral deposition, dentin production etc. In some embodiments, quantifiable parameters include differentiation, survival and regeneration of the stem cell-derived odontoblasts.

A plurality of assays comprising stem cell-derived odontoblasts can be run in parallel with different agent concentrations to obtain a differential response to the various concentrations. As known in the art, determining the effective concentration of an agent typically uses a range of concentrations resulting from 1:10, or other log scale, dilutions. The concentrations can be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the stem cell-derived odontoblasts (or organoids comprising such odontoblasts) used in the screen can be manipulated to express desired gene products.

### Kits

In some embodiments, the compositions as described herein can be prepared as a kit. In another embodiment, the kit further comprises an iPS cell or ES cell preparation, which can be metabolically active or frozen, and can optionally include reagents as described herein for differentiating cells from the iPS cell or ES cell preparation to an odontoblast phenotype.

In another embodiment, the kit comprises stem cell-derived odontoblasts, which can be metabolically active or frozen. In another embodiment, the kit and/or any of its constituents can be shipped and/or stored at ambient or room temperature, or at, e.g., 4° C.

In some embodiments, the iPS cells, ES cells, or stem cell-derived odontoblasts are human cells, rodent cells, canine cells, and the like.

### EXAMPLES

### Example 1

### DETAILED DESCRIPTION

In order to bioengineer missing tooth structure with naturally produced dentin, the following elements are required: a stem cell source, a scaffold, a nutrient source, and small molecule growth factors to direct signaling pathways²².

The dental ectomesenchyme is rich in stem cell sources, including dental pulp stem cells (DPSC) and stem cells of the apical papilla (SCAP). DPSC have previously been shown to successfully differentiate towards osteogenic and odontogenic fates²³⁻²⁵ and have been characterized in detail²⁶. Unfortunately, DPSC expansion and regeneration capacity is limited²⁷, showing a dramatic decrease in regenerative capacity with increased age²⁸, and are lost in the case of pulpal necrosis. SCAP are a unique stem cell population present at the tip of the developing tooth root and are hypothesized to give rise to the primary odontoblasts that produce root dentin²⁹. SCAP are able to differentiate to odontoblasts in vitro³⁰. However, SCAP are only present during root development and therefore are not a viable stem cell source in adults³¹.

Thus, a large hurdle to developing a clinically translatable method of odontoblast regeneration and tooth organoid formation is the source of stem cells. Human induced pluripotent stem cells (HiPSC) were discovered by Shinya Yamanaka, who found that he could reprogram mature somatic cells to pluripotency by treating them with 4 transcription factors-Oct4, Sox2, Klf4, and Myc-now globally recognized as OSKM factors³². HiPSC are capable of giving rise to all cell types of the three germ layers. Thus, they represent a single source of cells to be used to replace those lost to damage or disease. HiPSC are self-renewing, and therefore represent an inexhaustible source of stem cells. HiPSC are generated from adult somatic cells and therefore avoid the ethical concerns of embryonic stem cells. HiPSC eliminate the risk of immune rejection as cells can be derived from an individual patient. HiPSC therefore provide an excellent source of stem cells for regenerative dentistry.

A scaffold is necessary for cells to adhere to and produce their extracellular matrix, as well as provide cell-respective morphology for cell maturation²². Dental scientists have explored various biomaterials (e.g. collagen, hyaluronic acid, alginate), with recent studies indicating polylactic acid-co-polyglycolic acid (PLGA) polymers to be the primary scaffold selection for dental regeneration due to high porosity and open structure, resulting in increased cell adherence; biodegradable nature; and successful proliferation and differentiation of seeded DPSC³³⁻³⁶.

Blood vessels serve the critical role of delivering nutrients and oxygen to tissues, as well as removing waste products. Revascularization of the dental pulp can be promoted in immature teeth by inducing bleeding at the root apex, allowing influx of angiogenic cells³⁷. However, this method is not successful in necrotic adult dentition, likely due to a lack of stem cell populations able to respond to cell homing. The lab has dissected the Angiopoietin signaling that guides angiogenesis, finding the application of computationally designed protein scaffolds induced revascularization following injury³⁸⁻⁴⁰. This finding holds much promise in application for dental pulp revascularization.

Odontogenesis involves sequential, reciprocal signaling between a diverse cell population originating from the dental epithelium and underlying ectomesenchyme⁴¹. It has been well studied in the mouse incisor that five highly conserved pathways are active throughout tooth development: fibroblast growth factor (FGF), bone morphogenic protein (BMP), sonic hedgehog (HH), wingless-related integration site (WNT), and ectodysplasin (EDAR)⁴²⁻⁴³. For example, the mesenchymal signaling molecule BMP4 regulates the bud-to-cap stage transition. Absence of mesenchymal BMP4 arrests tooth development at the bud stage, while addition of exogenous BMP4 rescues the bud-to-cap transition⁴⁴. FGFR2 knockout mouse models show tooth development arrests at the bud stage⁴⁵⁻⁴⁶, indicating that FGF signaling is required for normal tooth development. FGF robustly stimulates cell proliferation and cytodifferentiation during tooth morphogenesis, with FGF4 ligand shown to be highly specifically expressed in murine enamel knot⁴⁷. Interestingly, in the primary human dentition, FGF4 has broad expression in both the epithelial and dental ectomesenchyme derived tissues, highlighting the variation between murine and human development⁴⁸. SHH is highly expressed in the inner enamel epithelium and ameloblasts⁴⁹, with expression shifting to the odontoblast once dentin is deposited⁵⁰, suggesting a role for HH signaling in odontoblast maturation.

The majority of studies on the signaling pathways controlling tooth development have been performed on the mouse incisor, which constantly renews lost tissue due to stem cell populations, and therefore is not a direct comparative model for human tooth development. The intricate intercellular molecular signaling that regulates human odontoblast development and maturity, and clear markers for the stages of human odontoblast differentiation, remain unknown. Thus, the largest barrier posed to regenerating odontoblasts and dentin tooth structure is the lack of knowledge on the molecular signaling involved in human odontoblast differentiation and the biological markers of odontoblast cells as they mature.

### I.V Single Cell RNA Sequencing of the Developing Human Tooth can Provide the Missing Signaling Knowledge Needed for Odontoblast and Dentin Regeneration.

Single cell RNA sequencing allows for identification of specific cell types within a heterogeneous population and dissection of their lineage projection on a molecular level⁵¹. Importantly, this ability to discriminate between cell populations has allowed for identification of novel odontoblast markers in the mature human adult dental pulp⁵² and dental epithelium of the chronically growing mouse incisor⁵³, as well as shown conserved gene expression profiles between stem cells of the dental pulp and periodontal ligament, suggesting functional differences are due to environmental niche cues⁵⁴. Due to the rarity of developing human tissue, analysis of the developing oral cavity at single cell resolution is missing, leaving the transcriptomic identities of dental ectomesenchyme derived-cells and the transcriptional effectors that specify progenitor fate and odontoblast differentiation unknown.

I.VI Existing In Vitro iPSC Derived Odontoblast Differentiation Protocols are Lacking.

As the need for odontoblast regeneration is critical, previous studies have explored stem cell-derived odontoblast differentiation protocols. In animal models, odontoblast-like cells have been produced from murine iPSC through co-culture with dental epithelium, with the goal of mimicking early tooth development in which the odontoblasts are in close proximity with the ameloblast⁵⁵⁻⁵⁶; and through gene transfection of iPSC to increase BMP4 and PAX9 expression⁵⁷. Both these methods are impractical for therapeutic application as access to developing human oral epithelium is limited and human gene therapy requires further study of off target effects. Interestingly, a recent study found supplementation of murine iPSC derived neural crest like cells with BMP4, FGF8 and WNT3a increases expression of odontoblast marker genes and odontoblast-like morphology⁵⁸. In humans, Xie et al produced iPSC-derived odontoblast-like cells through BMP4 supplementation⁵⁹. Unfortunately, the authors failed to transition through a neural crest state prior to odontoblast differentiation. This gap in odontoblast development misses a crucial stage in odontoblast differentiation, preventing full analysis of odontoblast development and disease modeling. The need for an efficient HiPSC derived odontoblast differentiation protocol remains for tooth regeneration and tooth development studies.

I.VII Beyond Dental Regeneration, In Vitro iPSC Odontoblast Differentiation Serves as a Tool for Disease Modeling and Developing Therapeutics.

A method of producing functional odontoblasts from HiPSC has applications that extend beyond bioengineering lost tooth structure. This tool serves as a model essential to study genetic diseases affecting dentin formation, such as Tricho-Dento-Osseous (TDO) syndrome, as well as develop clinically translational therapies. TDO is a rare but highly penetrant autosomal dominant disorder associated with mutations in the homeodomain transcription factor gene DLX3⁶⁰. Individuals with TDO suffer from ectodermal dysplastic defects in hair, teeth, and bones⁶¹⁻⁶². TDO produces debilitating dental defects leading to increased dental caries and fracture, resulting in high risk of pulpal necrosis and tooth loss. During early tooth development, Dlx3 is expressed in cranial neural crest cells of the branchial arches⁶³. Loss-of-function studies in mice show that Dlx3 directly regulates transcription of Dspp by binding to the promoter region; DLX3 knock out mutants exhibit downregulation of Dspp and dentin defects⁶⁴. Cytodifferentiation of odontoblasts in mouse DLX3 knock-out models is disrupted, leading to impaired dentin production and odontoblast apoptosis⁶⁵. Interestingly, DLX3 inhibits proliferation of human dental pulp cells and has been proposed to play a role maintaining quiescence of this cell population⁶⁶. The role of DLX3 in human odontoblast maturation has yet to be fully explored due to a lack of a developing human odontoblast model. In order to develop TDO therapies, it is critical to deepen the understanding of DLX3's role in human odontoblast development.

### Materials & Methods

### II.I Single Cell RNA Sequencing of the Developing Human Oral Cavity.

Human fetal tissue collection and dissection. Fetal cranial facial tissues were provided by the Birth Defect Research Laboratory University of Washington after obtaining an informed consent from the patient or legal guardian. This study was approved by the Human Subjects Division of the University of Washington (HSD #51634-EJ) and all methods were performed in accordance with the relevant guidelines and regulations.

The tooth and salivary gland samples were collected from five fetal age groups representing the following developmental stages for tooth differentiation: the bud stage (gw9-11), the cap stage (gw12-13), the early bell stage (gw14-16), and the late bell stage (gw17-22)^{42,67}. Tissues were transferred from the BDRL submerged in Hank's Balanced Salt Solution media (Gibco 14025092) on ice. The tissues were further dissected under a dissection microscope to isolate tooth germs, or salivary glands, while still submerged in cold RNase free Phosphate-Buffered Saline (PBS) (Invitrogen AM9624) within six hours from the initial dissection at BDRL. Samples that exceeded that time were excluded from single cell analysis and instead used for histology and immunostaining. Extracted tissues were transferred into Eppendorf tubes and snap frozen using liquid nitrogen. The frozen samples were stored at −80° C. until nuclei extraction.

Single cell RNA sequencing. Single cell combinatorial indexing RNA sequencing (sci-RNA-seq) was performed in collaboration with the Brotman Baty Institute and has been described in detail previously⁶⁸. Briefly, cells undergo split-pool barcoding to uniquely label each cell within the entire population of single cells. The first level of indexing occurs as permeabilized nuclei are distributed across a 96-well plate, then each well receives a specific unique molecular identifier (UMI) incorporated through reverse transcription, barcoding each cell within the well. UMI labeled cells are then pooled and redistributed to multiple 96-well plates for introduction of a second well-specific identifier incorporated through PCR amplification. Amplicons are then pooled for parallel sequencing, producing a transcriptomic library composed of cells identified by their unique combination of barcodes.

sci-RNA-seq data processing. Low quality reads were removed by filtering UMI reads higher than 1500 and lower than 100, followed by removal of all mitochondrial reads. Utilizing the Monocle 3 workflow described previously⁶⁹⁻⁷⁰ data underwent normalization by size factor, preprocessing, dimension reduction into Uniform Manifold Approximation and Projection (UMAP) space⁷¹, and unsupervised clustering producing grouping of the cells into clusters based on similarity of gene expression, as done previously⁶⁸.

Dental ectomesenchyme derived cell population identification. Epithelial and mesenchymal derived cells of the anterior jaw and incisor tooth germ were identified by expression of oral epithelium specific markers KRT5⁷² and PITX2⁷³ and dental ectomesenchyme specific markers PRRX1⁷⁴ and RUNX2⁷¹. The developing jaw ectomesenchyme, dental ectomesenchyme, and odontoblast cells were subset to identify the dental ectomesenchyme derived cell types.

Heatmap and Gene Ontology (GO) terms enrichment. The package ComplexHeatmap⁷⁶ was used to generate custom heatmaps that integrate Gene Ontology (GO)-terms for each cluster. GO-terms were generated using the ViSEAGO package⁷⁷, which utilizes the top 50 marker genes per cluster as input to determine associated GO-terms. The GO-terms were sorted by p-value, the top 100 selected, and keywords extracted via simplifyEnrichment package⁷⁸. Keywords were filtered to eliminate redundant and irrelevant terms. Age Score is calculated by normalization of the cell count per time point.

Pseudotime and real-time analysis. Cell lineage trajectories and pseudotime were produced using Monocle 3. Pseudotime is calculated from dynamic changes in differentially expressed genes (DEG) and defines a cell's progress along the developmental trajectory⁶⁹.

Cell cycle scoring. Cells were categorized into cell cycle phase according to expression of G2/M and S phase markers as described previously⁷⁹.

Regressing out the cell cycle effect. The cell cycle effect was regressed out as described previously⁸⁰ by simple linear regression using Seurat⁸¹.

Identification of critical signaling pathways via Top Pathway analysis. The lab has developed a comprehensive analysis pipeline to evaluate signaling pathway activity based on ligand-receptor interactions and downstream activity. Prior to analysis, a differentiation trajectory with known progenitor, maturely differentiated target cell, and neighboring support cells present at the same developmental stage should be known. Dental epithelium derived cells were included as sources of ligand producing cells. This pipeline utilizes the talklr packages² to identify and rank incoming ligand signals to the progenitor cell of interest (e.g. dental papilla and preodontoblast), filtering for ligand-receptor interactions associated with major signaling pathways of interest (e.g. FGF, BMP, HH). Each interaction is assigned a normalized interaction score, which is calculated by dividing the sum of interaction scores across all pairwise cell-cell interactions. The DEsingle package⁸³ was utilized to produce the DEG between the progenitor and target cells (False Discovery Rate <0.1 and Fold-Change >2). scMLnet package was used to generate a multilayer network modeling the upstream ligand-receptor pairs (from talklr) and downstream transcription factors (TF) and their target genes (from DESingle). Connectivity of each layer of the model was scored to predict which pathway is the most active. Scores were calculated by determining target gene fold-change; mean TF-target genes associated with a given TF; sum of TFs associated with a given receptor; sum of receptors associated with a given ligand; and finally sum of ligands that are associated with a given signaling pathway. Score normalization is performed at each layer. Finally, the pipeline ranks signaling pathways by activity score, indicating the most active pathways including the key drivers of differentiation between progenitor and target maturely differentiated cells.

### II.II Validation of Sci-RNA-Seq Computational Findings In Situ in the Human Tooth Germ.

Crysectioning of developing human tooth germs. tooth germs embedded in optimal cutting temperature compound were cryosectioned to 10 μm sections. Following sectioning, slides were stored at −80° C. and warmed at room temperature prior to staining.

RNAScope assay and confocal imaging. RNAScope assay was performed as described in detail previously using the RNAScope HiPlex12 Reagent Kit v2 (ACD)⁸⁴. Briefly, a 12-probe RNAScope HiPlex assay (Advanced Cell Diagnostics, Inc.) including probes against 13 transcripts differentially expressed between cell type clusters in ectomesenchyme- and epithelial-derived lineages were selected to distinguish cell populations: VWDE, SALL1, FGF4, IGFBP5, FGF10, PRRX1, FBN2, ENAM, PCDH7, SOX5, KRT5, and either DSPP or LGR6. (Table 1). Cryosectioned tooth germ sections were fixed for 1 hour in 4% PFA at room temperature then rinsed with PBS. Sections were dehydrated by sequential treatment with 50%, 70% and 100% ethanol, followed by permeabilization via Protease IV to allow probe access. Probes were then hybridized by incubation in the HybEZ Oven for 2 hours at 40° C., and rinsed twice in 1× Wash Buffer for 2 minutes. Following hybridization of the probes, the signal was amplified by sequential incubation of RNAscope HiPlex Amp 1-3, each amplification for 30 minutes at 40° C. Autofluorescence was reduced by treatment with Formalin-Fixed Paraffin-Embedded Reagent for 30 minutes at room temperature. HiPlex Fluoro T1-T4 were then hybridized for 15 minutes at 40° C. Nuclei were stained using DAPI and slides were mounted using ProLong Gold Antifade Mountant. All incubations not performed at room temperature were done via the HybEZ Oven.

TABLE 1
RNAScope Probe Sequences.

| Gene | Cluster | Relative expression |
|---|---|---|
| SALL1 | SOB | 1.603378639 |
| FGF4 | EK | 2.072977626 |
| IGFBP5 | SOB | 2.0953 |
| FGF10 | DP | 2.342748687 |
| PRRX1 | DEM | 2.394496229 |
| FBN2 | POB | 2.405502878 |
| PCDH7 | SI-2 | 2.478646553 |
| SOX5 | DP | 2.767965189 |
| KRT5 | PAN-DE | 2.805817869 |
| VWDE | AM | 2.826717442 |
| DSPP | OB | 3.019020124 |
| ENAM | SAM | 6.316622834 |

RNAScope imaging and analysis. The first four probes were imaged after completing the manufacturer's specified hybridization steps, counterstaining, and coverslipping. Images of tissue sections were obtained using an Nikon Ti2 with an Aura light engine (Lumencor, Beaverton, OR), and BrightLine Sedat filter set optimized for DAPI, FITC, TRITC, Cy5 & Cy7 (Semrock, Rochester, NY: LED-DA/FI/TR/Cy5/Cy7-5X5M-A-000). Coverslips were removed, the first four fluorophores were cleaved, and the process was repeated for probes 5-8 and then probes 9-12. Images were analyzed using Fiji (ImageJ2 v2.3.0) and QuPath (v0.3.0) quantitative pathology and bioimage analysis freeware⁸⁵. Briefly, The DAPI channel images for imaging rounds two and three were aligned to the DAPI image for imaging round one using the BigDataViewer>BigWarp plugin in Fiji. Matching reference points were identified across the DAPI images and the resultant landmark tables were used in a custom groovy script to align the FITC, Cy3, Cy5, and Cy7 images from the three rounds of imaging. Images were uniformly background corrected and scaled. Cellular segmentation was performed in QuPath and positive signal foci and clusters were identified as subcellular detections. Parameters were set to allow for detection of foci while avoiding false positive detection events using positive and negative control images. From QuPath, the coordinates and the number of spots estimated (sum of individual puncta and estimated number of transcripts for clustered signal) for each segmented cell were processed using custom R scripts to map cell locations and expression levels. Out of the transcripts assayed by RNAScope, probe set criteria used to identify a given cell population in RNAScope data was selected based on differential expression across the cell types identified in the sci-RNA-seq data at corresponding time points. Cells matching expression criteria for a cluster's probe set were designated by cluster color and mapped spatially.

Immunofluorescence protein staining and confocal imaging. Tissue sections and cultured cells were fixed in 4% paraformaldehyde (PFA) then immersed in 1× phosphate-buffered saline (PBS) for 3×5-minute washes. Antigen retrieval was performed using 10× Citrate Buffer (Sigma-Aldrich) in a capped coplin jar microwaved for −45 seconds followed by 15-minutes incubation in the microwave. Slides were then washed in PBS at room temperature for 7 minutes. Slides were blocked for 90 minutes at room temperature in a humidified chamber with a blocking buffer consisting of 0.1% Triton X-100 and 5% Bovine Serum Albumin (BSA) (VWR). The primary dentin sialophosphoprotein (DSPP, Santa Cruz Biotechnology), ameloblastin (AMBN, Santa Cruz Biotechnology), and amelogenin (AMELX, Santa Cruz Biotechnology) antibodies were used at a 1:50 concentration, in conjunction with the primary keratin 5 antibody (KRT5) at 1:100. The primary AP-2α transcription factor (AP-2α, Abcam) was used in conjunction with the primary Nerve growth factor receptor (p75 or CD271, ThermoFisher) at a 1:500 concentration. The primary antibodies were incubated overnight at 4° C. in a humidified chamber. After 3×5-minute washes in PBS in a coplin jar, the slides were transferred to a humidified chamber with secondary antibodies. Secondary antibodies goat anti-rabbit 568 and goat anti-mouse 488 (1:250, Molecular Probes) were applied for 75 minutes at room temperature in the same blocking agent. Slides were then rinsed with PBS 4×10-minute washes in a coplin jar. DAPI (1:50, Molecular Probes) was applied for 10 minutes at room temperature in PBS. Slides were then rinsed with PBS for 10 minutes in a coplin jar. Slides were then mounted with Vectashield (Vector Labs) and stored at 4° C. until used for imaging. Confocal Imaging was done on a Leica TCS-SPE Confocal microscope using a 40× objective and Leica Software. Images were processed with Fiji software distribution of ImageJ v1.52i⁸⁴⁻⁸⁵. Negative controls were performed substituting PBS.

### II.III Human Induced Pluripotent Stem Cell (HiPSC) Culture and Differentiation.

HiPSC culture. HiPSCs line WTC-11 (Coriell, #GM25256)⁸⁸⁻⁸⁹ are seeded on 6-well plates and cultured in mTeSR1 stem cell medium (StemCell Technologies, #85850) with daily media changes until cells reach ˜70% confluency as described previously⁹⁰. mTeSR is a feeder-free maintenance media designed to support a pluripotent state by including key molecules insulin to promote cell survival and proliferation, bFGF for self-renewal and expansion, and TGF to inhibit reprogramming⁹¹. ROCK inhibitor (ROCKi) (Stemcell Technologies) is added to mTesR for initial 24-hours. To maintain HiPSCs and prevent fusing or premature differentiation of colonies, regular observation under low-power microscopy will be performed with colony passaging as necessary.

HiPSC derived neural crest differentiation (iNC). This project applies the protocol previously described to produce iPSC derived neural crest (iNC) through dual SMAD inhibition and early WNT activation⁹² (FIG. 5A). HiPSC are seeded at 32,000 cells per well on 6-well matrigel coated plates and maintained in mTeSR until 70% confluent. Differentiation is induced (Day 0) with addition of Basal Neural Maintenance Media (BNMM), which consists of 250 mL DMEM/F12+glutamine (Gibco 11320-033) and 250 mLneurobasal media (Gibco 21103-049) supplemented with 2.5 mL N2 (Gibco 17502-048), 5 mL B27 (Gibco 17504-044), 2.5 mL GlutaMax (Gibco 35050-061), 2.5 mL ITS-A (Gibco 51300-044), 400 μL 2-Mercaptoethanol (Thermo Fisher 21985023), and 2.5 mL NEAA (Thermo Fisher 11140050). On Day 0, BNMM is supplemented with 10 μM SB 431542 (Biogems BG6675SKU301) and 1 μM LDN 193189 (Biogems BG5537SKU106) for dual SMAD inhibition; inhibition is maintained until Day 4 and Day 3 respectively. On Day 2, WNT is activated via supplementation with 3 μM CHIR 99021 (Tocris Bioscience 4423), which is maintained until Day 11. Media change occurred daily.

Magnetic cell sorting for p75+ iNC. On Day 11, cells were lifted via Accutase (Sigma-Aldrich A6964) and resuspended in an IMAG buffer consisting of 0.5% bovine serum albumin and 2 mM EDTA. iNC were isolated with addition of PE conjugated p75 (also known as Nerve Growth Factor Receptor, NGFR, and CD271) antibody (Thermo Fisher 12-9400-42) and Anti-R-PE magnetic beads (BioSciences 557899). p75+ cells were eluted, resuspended in media, and plated on 24-well matrigel coated plates at a density of 250,000 cells per well.

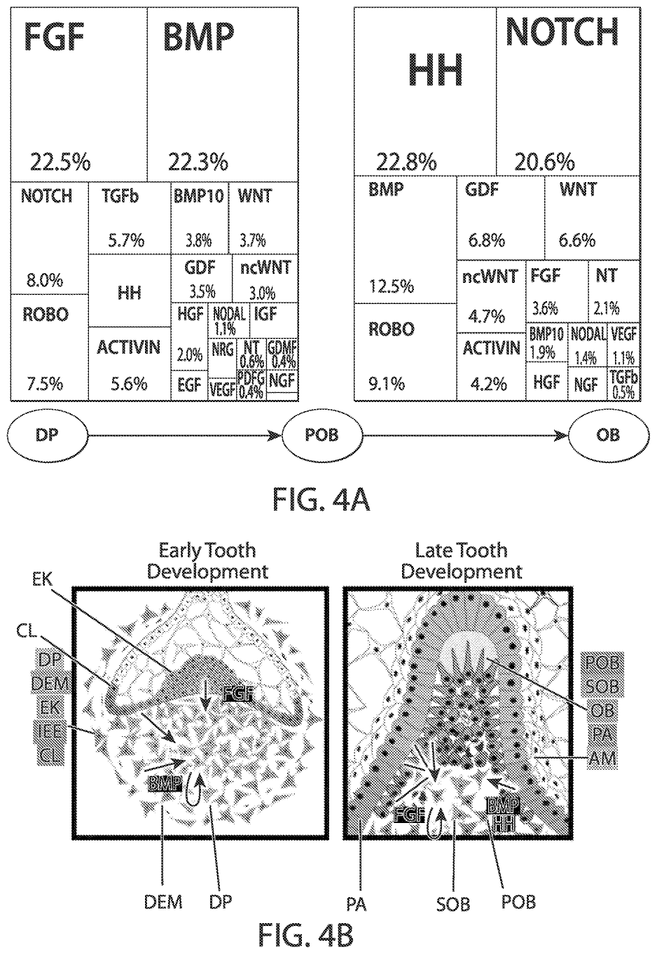

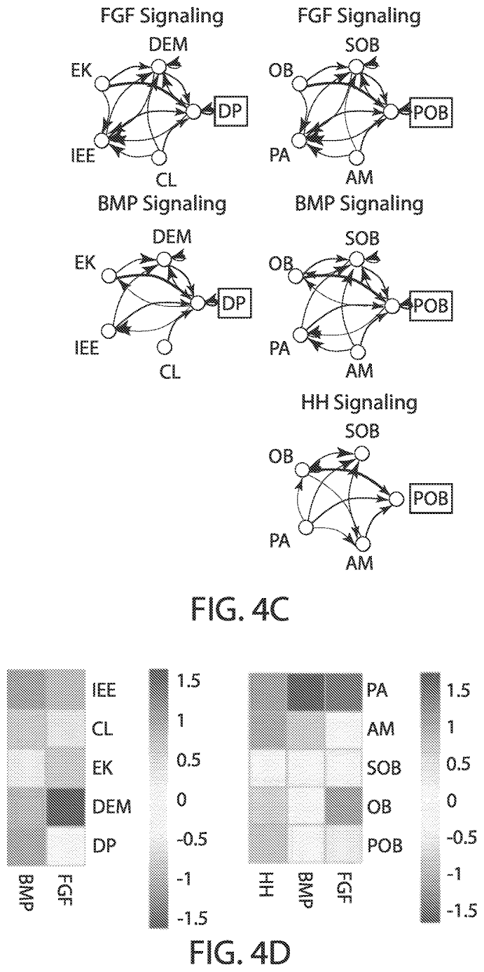

iNC derived odontoblast differentiation (00B). This project applies the DPSC-derived iPSC to odontoblast protocol previously described by the lab²⁶, modified to reflect the full signaling pathway activities as detected by the sci-RNA-seq analysis (FIG. 4A; FIG. 5E). p75+ iNC were cultured in Odontogenic Medium (Day 12), which consists of DMEM+Glutamax (Gibco 10566016), 100 nM dexamethasone (Sigma-Aldrich D4902), 10% fetal bovine serum, 5 mM b-glycerophosphate (Sigma-Aldrich G9422), and 50 μg/mL L-ascorbic acid (Sigma-Aldrich A4544). Media was supplemented with 100 ng/mL BMP4 (Stemcell Technologies 78211) and either basic FGF (bFGF or FGF2), FGF8b, or FGFR minibinders C1 or C6 for 8 days (D0-D7), followed by 50 ng/mL BMP4 and 400 nM SAG (Stemcell Technologies 73412) for 7 days (D8-D14).

All cultures were performed on Matrigel coated plates at a 1:30 dilution and incubated at 37° C. and 5% CO₂ concentration. Each differentiation was performed in triplicate with undifferentiated HiPSC as negative control.

Real-time quantitative reverse transcription-polymerase chain reaction (OPCR). To analyze gene expression, cells were dissociated and lysed with Trizol (Life Technology) with cell pellets stored at −80° C. RNA purification is performed via TURBO DNA-Free™ Kit (Invitrogen) or Aurum™ Total RNA Mini Kit (Bio-Rad), purity and concentration quantification via Nanodrop ND-1000 (Thermo Fisher Scientific). cDNA synthesis via iScript™ cDNA Synthesis Kit (Bio-Rad) or Applied Biosystems™ High-Capacity cDNA Reverse Transcription Kit (Thermo Fisher Scientific), and QPCR performed using oligonucleotide primers for neural crest and odontoblast markers (Table 2), SYBR Green reporter (Applied Biosystems) and 7300 Real-Time PCR System (Applied Biosystems). All QPCR reactions were performed in triplicate, normalized to β-actin and HiPSC, and assessed using the comparative change in threshold cycle (DDC) method. β-actin primers are SEQ ID NOs: 9 and 10; SOX10 primers are SEQ ID NOs: 11 and 12; PAX3 primers are SEQ ID NOs: 13 and 14; MSX1 primers are SEQ ID NOs: 15 and 16; DSPP primers are SEQ ID NOs: 17 and 18; S100A13 primers are SEQ ID NOs: 19 and 20.

TABLE 2
QPCR Primer Sequences.

| | | Forward | Reverse |
|---|---|---|---|
| | | Sequence | Sequence |
| Cell Type | Primer | (5¹→3¹) | (5¹→3¹) |
| Endogenous | β-ACTIN | TCCCTGGAGAAG | GTAGTTTCGTGG |
| control | | AGCTACG | ATGCCACA |
| Neural | SOX10 | CTGGACACTAAA | CATGTGGACTGG |
| crest | | CCCCTGCC | GCTGCAGAC |
| | PAX3 | TGTTTCCCTTTC | TTATATCGCCTT |
| | | CTGTGTGG | GGGCATTG |
| Odontoblast | MSX1 | CTCGTCAAAGCC | CGGTTCGTCTTG |
| | | GAGAGC | TGTTTGC |
| | DSPP | GAGCAACACGGA | CACTCTTGTCAC |
| | | TGGATGATTTC | GCACAGCCTTA |
| | S100A13 | TTGAACTCGTTG | GCAGAACCACTG |
| | | ACGCTGAGG | ACAGAGCTA |

Statistical analysis. DDCₜ values of gene expression of differentiated samples are compared to undifferentiated HiPSC cells (N=3) and analyzed for significance using Student's ttest via GraphPad QuickCalcs (graphpad.com).

Mineralization capacity assav. Alizarin red staining (ARS) (Sigma-Aldrich TMS-008) is performed to assess extracellular calcium deposition. Culture medium was aspirated from each well and cells washed with PBS 3×. Cells were fixed in 4% PFA for 15 minutes at room temperature. PFA was removed and cells washed 3× with diH₂O. diH₂O is aspirated off and 1 mL 2% ARS was added per well. Plates were incubated, covered in aluminum foil, at room temperature for 45 minutes with gentle shaking. ARS was removed and cells washed 5× with diH₂O. Staining was then visualized under phase contrast microscopy (Olympus IX70 microscope, Japan). Stain was released with 10% acetic acid and neutralized with 0.1 μM ammonium hydroxide. Stain quantification for OD405 was then performed via Wallac EnVision system.

### II.IV DLX3 Mutant Line Generation Using CRISPR-Cas9.

DLX3 knockout mutant line generation. Guide RNA (sgRNA) was designed to target the early coding region of the DLX3 gene. Ribonucleoprotein (RNP) complex was prepared by combining DLX3 sgRNA and the Cas9 enzyme. RNP was then delivered to HiPSC via Amaxa nucleofector⁹³.

Validation of Cas9 nuclease activity. Genomic DNA was harvested from mutant cells using DNAzol (Thermo Fisher 10503027) and amplification of the DLX3 target DNA was performed via Phusion Flash High-Fidelity PCR Master Mix (Thermo Fisher F548L) supplemented with 3% DMSO. Amplification of the DNA product was confirmed by gel electrophoresis. DNA product was isolated via Monarch Gel Extraction kit (NEB T1020S) and purified DLX3 DNA product was then assessed via Sanger sequencing (Genewiz).

Clonal isolation. Individual clones were isolated and screened for indel mutations resulting in a frame shift leading to a premature stop codon, leading to genetic knock-out of DLX3.

Total protein isolation. Media was aspirated and cells were gently rinsed with 1×PBS. Cells were lysed with 130 μl of lysis buffer containing 20 mM Tris-HCl (Sigma-Aldrich, 1185-53-1) (pH 7.5), 150 mM NaCl, 15% glycerol (Sigma-Aldrich, G5516), 1% triton (Sigma-Aldrich, 9002-93-1), 3% SDS (Sigma-Aldrich, 151-21-3), 25 mM β-glycerophosphate (Sigma-Aldrich, 50020-100G), 50 mM NaF (Sigma-Aldrich, 7681-49-4), 10 mM sodium pyrophosphate (Sigma-Aldrich, 13472-36-1), 0.5% orthovanadate (Sigma-Aldrich, 13721-39-6), 1% PMSF (Roche Life Sciences, 329-98-6), 25 U benzonase nuclease (EMD, 70664-10KUN), protease inhibitor cocktail (Pierce™ Protease Inhibitor Mini Tablets, Thermo Scientific, A32963), and phosphatase inhibitor cocktail 2 (Sigma-Aldrich, P5726), respectively, in a tube. Cell lysate was collected in a fresh Eppendorf tube. 43.33 μl of 4× Laemmle Sample buffer (Bio-Rad, 1610747) containing 10% beta-mercaptoethanol (Sigma-Aldrich, M7522-100) was added to the cell lysate and then heated at 95° C. for 10 min. The boiled samples were either used for Western blot analysis or stored at −80° C.

Western blot analysis. The protein samples were thawed and boiled at 95° C. for 10 min. 30 l of protein sample per well was loaded and separated on a 4-10% SDS-PAGE gel for 30 min at 250 Volt. The proteins were then transferred on a nitrocellulose membrane for 12 min using the semi-dry turbo transfer Western blot apparatus (Bio-Rad, USA). Post-transfer, the membrane was blocked in 5% bovine serum albumin for 1 h. After 1 h, the membrane was probed with the respective antibodies: DLX3 (Abcam, AB64953) at 1:500 dilution and β-Actin (Cell Signaling, 3700S) at 1:10,000 dilution. Membranes with primary antibodies were incubated at 4° C., overnight on a rocker. Next day, the membranes were washed with 1×TBST (3 times, 10 min interval). The respective HRP-conjugated secondary antibody (Bio-Rad, USA) at 1:10,000 dilution was added and incubated at room temperature for 1 h. All the membranes were washed with 1×TBST (3 times, 10 min of interval) after secondary antibody incubation and developed using Chemiluminescence developer and imaged using Thermo Scientific CL-XPosure Film or Bio-Rad ChemiDoc Imager.

### Results

### III.I Sci-RNA-Seq of Developing Human Teeth Identifies a Transcriptionally Heterogeneous Population of Dental Ectomesenchyme Derived Cells.

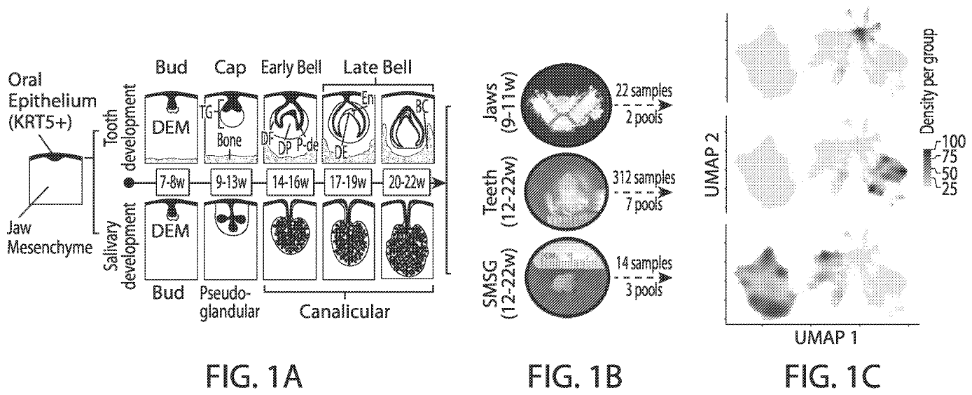

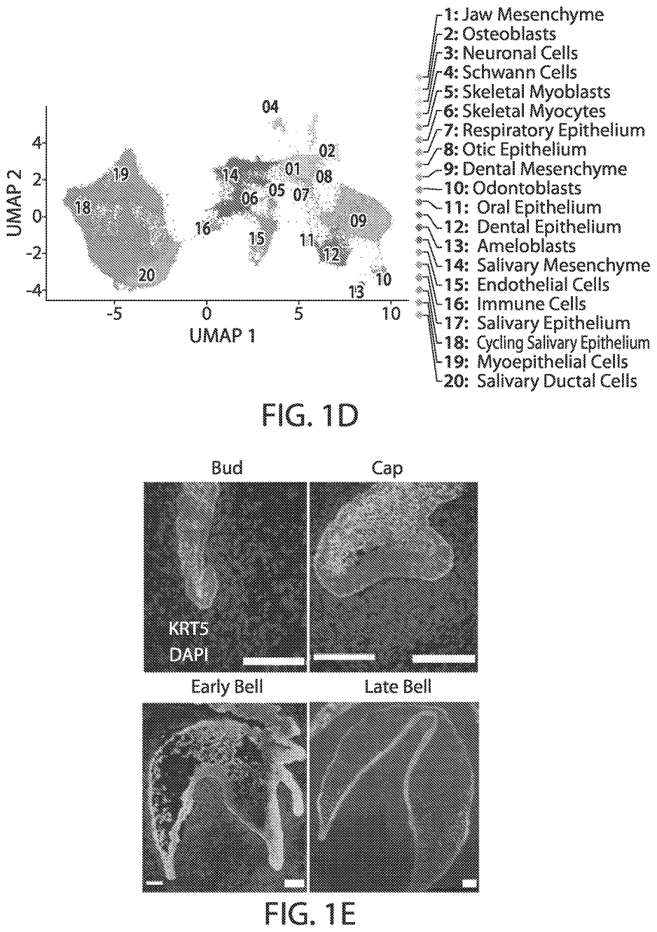

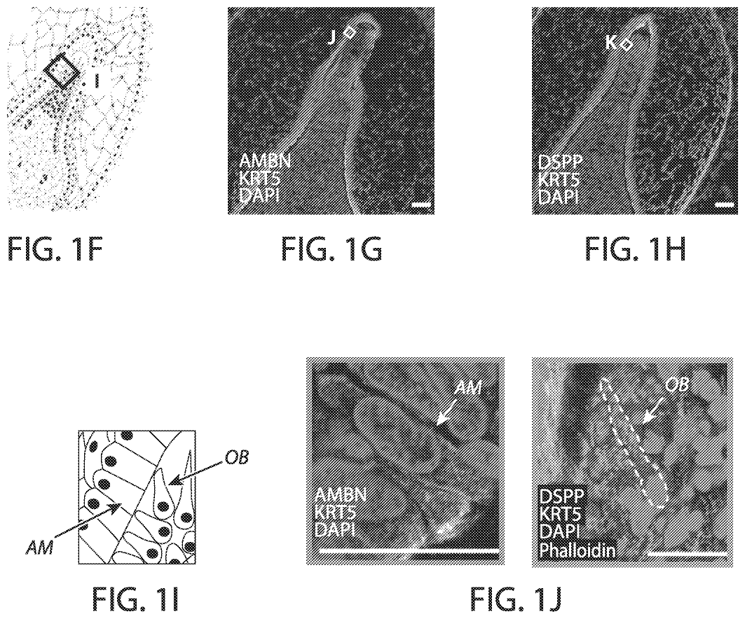

In humans, oral tissue development begins around 6 gw and starts as a thickening in the oral epithelium^{41-42,94}, giving rise to all primary teeth and salivary gland tissue. Individual teeth develop independently as an extension of the main dental lamina and progress through a series of morphological stages (bud, cap, & bell) within bony crypts of the jaws⁹⁵. Additionally, each developing tooth is surrounded by thick fibrous tissue called the dental follicle⁹⁶. The dental follicle and the tissue it contains comprise the tooth germ⁹⁷ (FIG. 1A). The oral epithelium will also give rise to the salivary glands (FIG. 1A). Like teeth, salivary glands derive from the invagination of a thickened sheet of epithelium into the underlying ectomesenchyme, known as the initial bud stage⁹⁸ (FIG. 1A).

To better understand early oral differentiation and to dissect how the mesenchymal cell lineages acquire odontogenic competence, the developmental gene expression profiles of human fetal stages were analyzed by single cell sequencing. tooth germ and salivary gland samples were collected from five fetal age groups (FIG. 1A-1B). These age groups represented the following developmental stages for tooth differentiation: the cap stage (9-13 gw), the early bell stage (14-16 gw), and the late bell stage (17-22 gw) (FIG. 1A-E)^{42,67}. Submandibular salivary glands were also collected from three matched timepoints (12-13 gw, 14-16 gw, 17-19 gw) that cover the pseudo-glandular and canalicular stages for salivary gland development⁹⁹ (FIG. 1A).

Single-cell sequencing data of the tissue samples were analyzed using Monocle3⁶⁸⁻⁶⁹ and visualized in uniform manifold approximation and projection (UMAP) space (FIG. 1D). The distribution of the cells from each tissue origin was identified by using density plots by tissue type (FIG. 1C). Utilizing a graph-based clustering algorithm, 20 major clusters were annotated based on key marker genes (FIG. 1E) from PanglaoDB. The major cell types in salivary gland samples include salivary mesenchyme, salivary epithelium, cycling salivary epithelium, myoepithelium, and ductal cells (FIG. 1C-ID). In the jaw samples (9-11 gw, FIG. 1C-ID) mesenchymal progenitors, osteoblasts, neuronal, Schwann cells, muscle, respiratory epithelium, otic epithelium and oral epithelium were identified (FIG. 1C-1D). The major cell types in tooth samples include dental ectomesenchyme and epithelium, and odontoblasts and ameloblasts respectively. The cell types observed in all samples include endothelial¹⁰¹⁻¹⁰³ and immune¹⁰⁴⁻¹⁰⁵ cells. The lab has characterized the developing human salivary gland in detail¹⁰⁶.

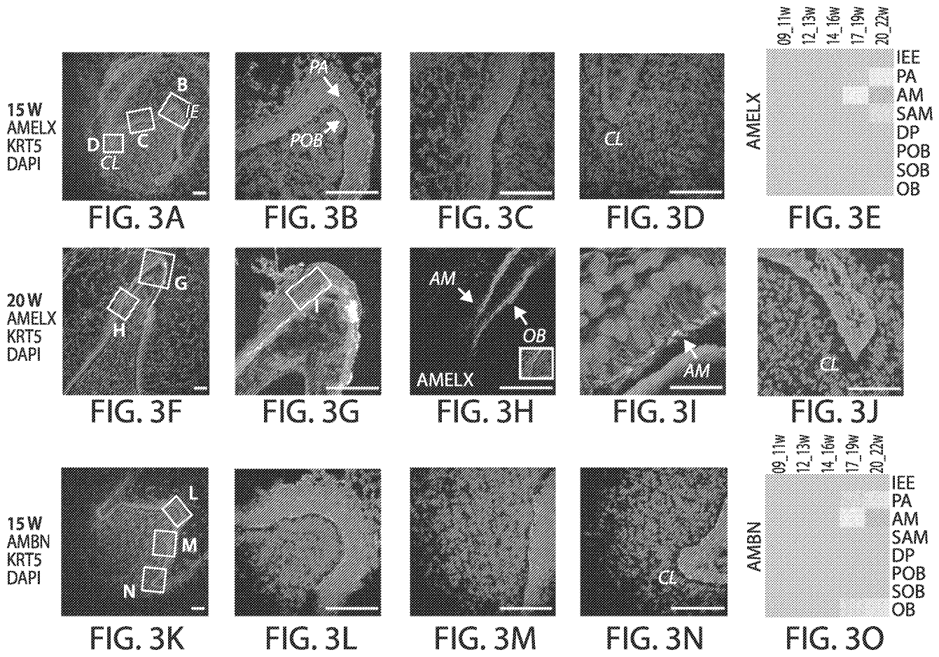

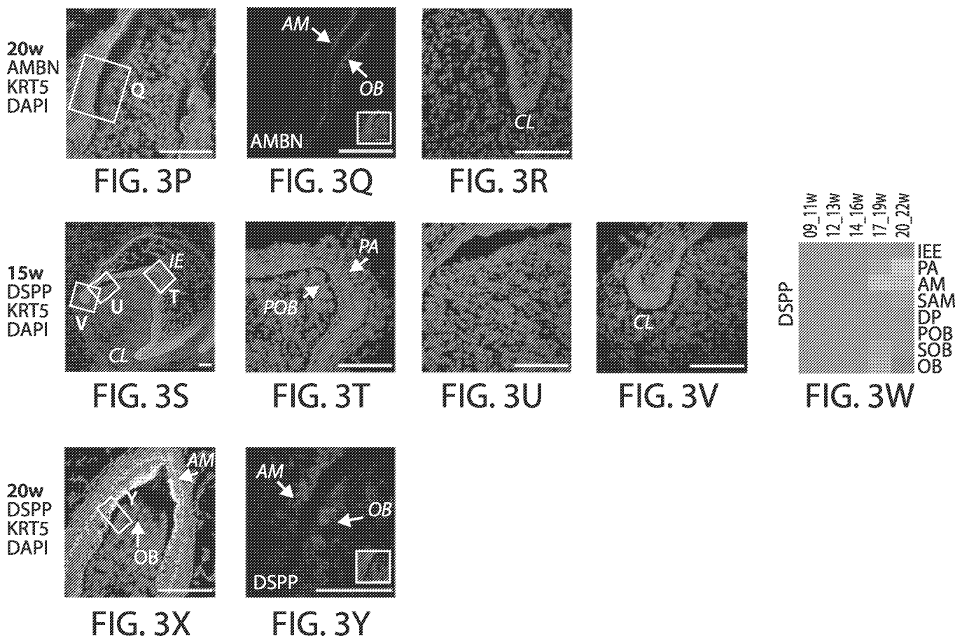

To confirm the timing of the tooth morphological stages, immunohistochemistry was performed on tissue sections, and as expected, all the dental epithelium derived tissues were visualized by KRT5 (FIG. 1E). There are two critical lineages in tooth development: odontoblasts and ameloblasts. These are the two cell types that secrete the mineralized protective layers that cover the soft dental pulp, which contains the nerves and the nutrient transporting blood vessels. Odontoblasts are ectomesenchyme-derived cells secreting the inner coverage for the pulp, called dentin, while ameloblasts are ectoderm-derived and secrete the outermost layer, enamel. To establish expression of known odontoblast and ameloblast markers in the tissue, immunohistochemistry was performed on human fetal tooth germ at 20 gw using dentin sialophosphoprotein (DSPP) and ameloblastin (AMBN) respectively (FIGS. 1F-1K, FIG. 3K-3Y). As expected, ameloblasts express AMBN in secretory vesicles (FIGS. 1I-1J), and likewise, odontoblasts secrete DSPP (FIG. 1I, 1K). Expression of ameloblast marker AMELX, observing a mirrored expression pattern was validated between the developing ameloblast and odontoblast (FIG. 3A-3J).

### III.II Sci-RNA-Seq of Developing Human Tooth Identifies a Transcriptionally Heterogeneous Population of Dental Ectomesenchyme Derived Cells.

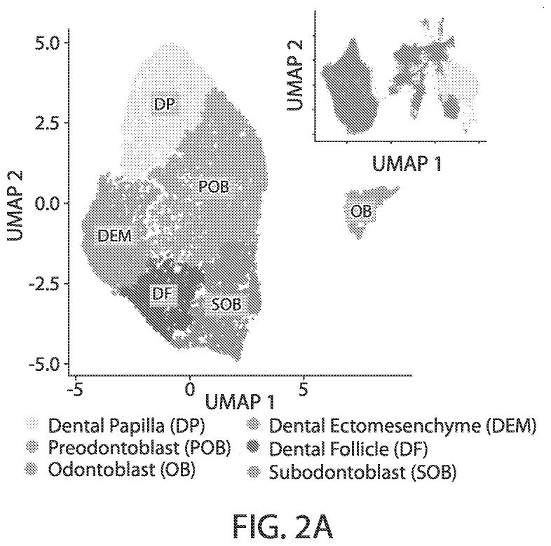

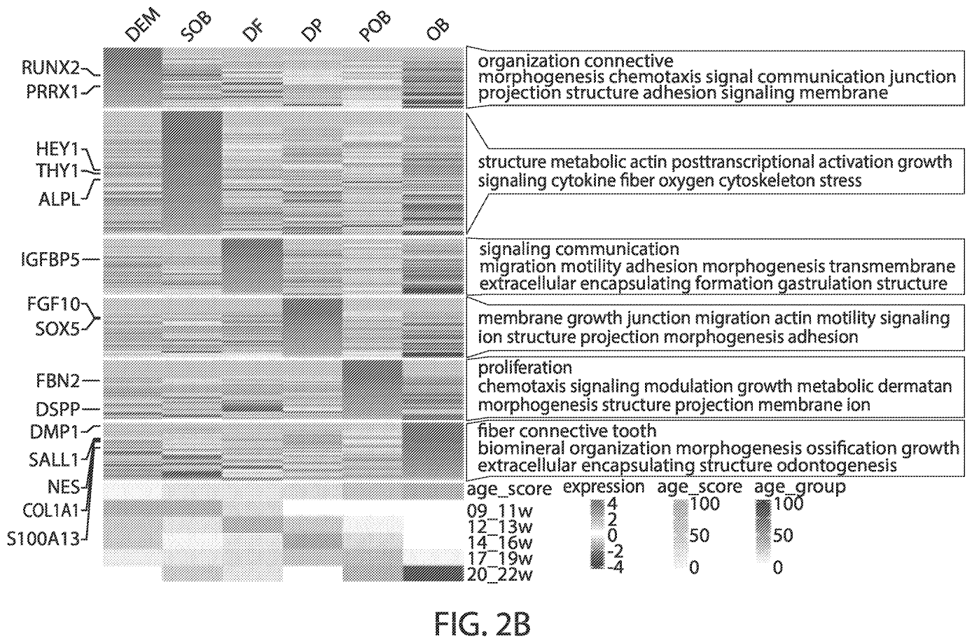

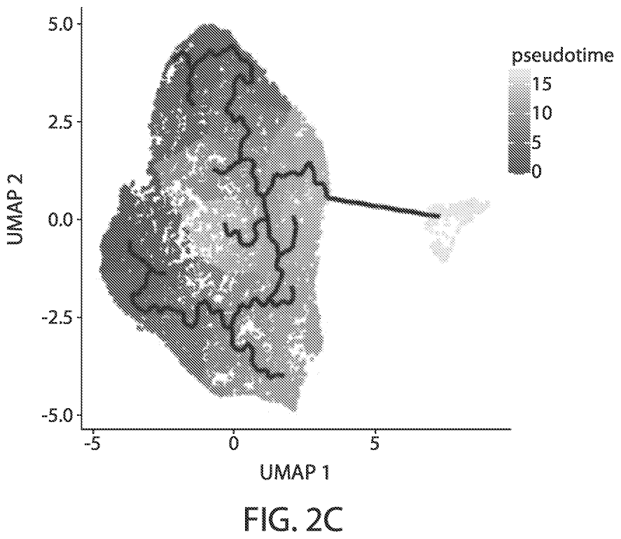

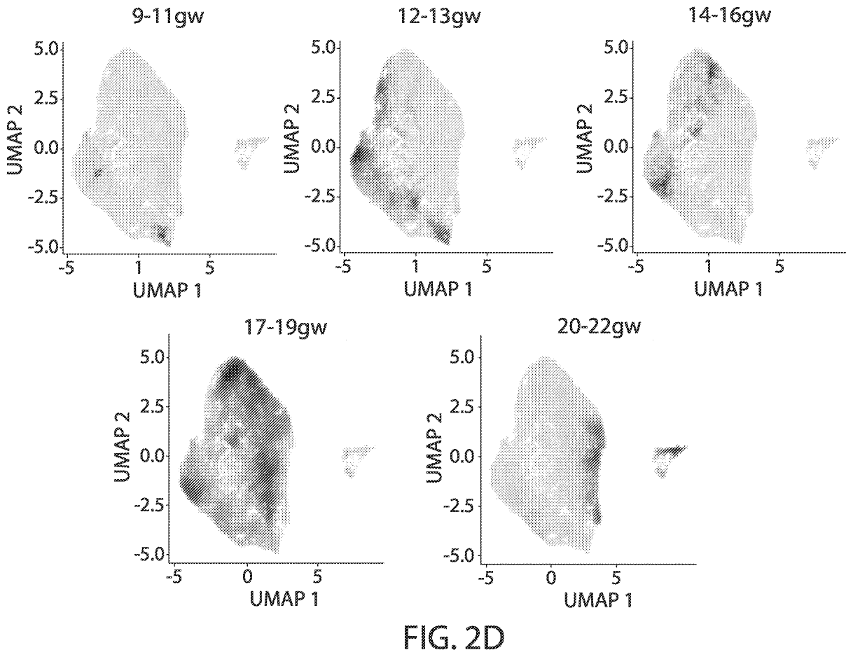

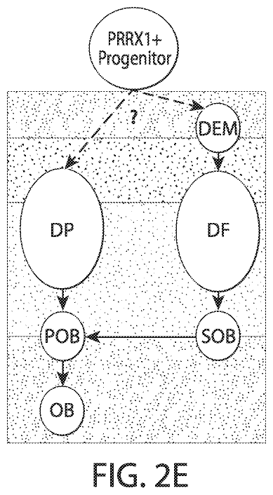

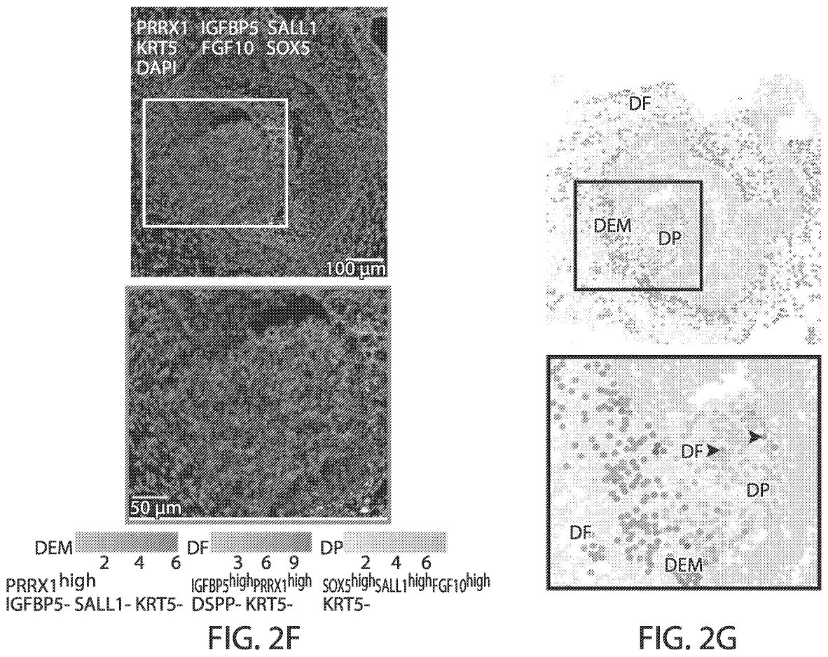

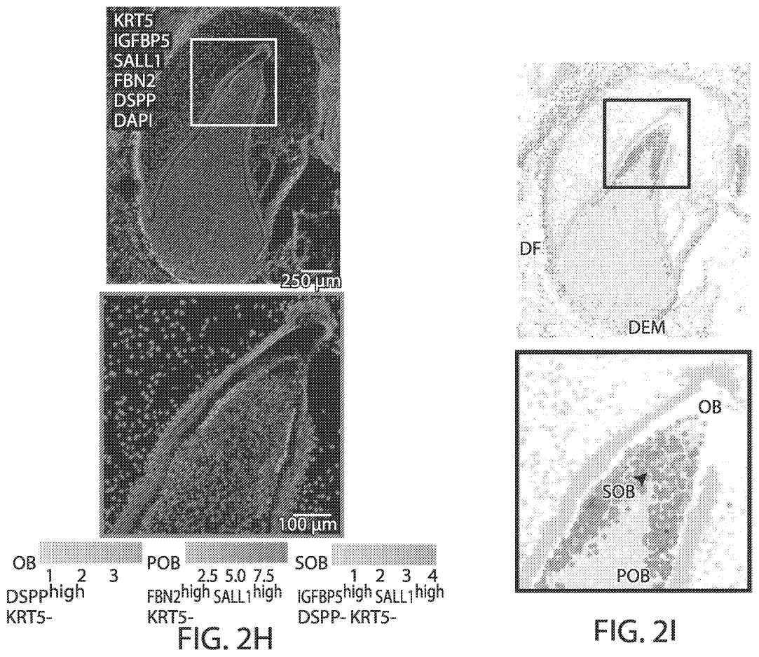

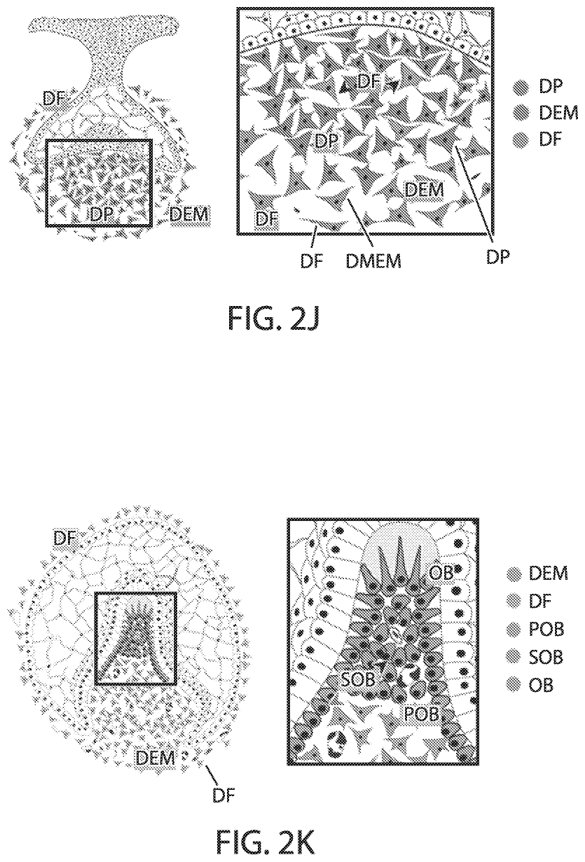

To dissect the cells derived from the developing jaw mesenchyme, dental ectomesenchyme, and odontoblast cells were identified by PRRX1, RUNX2 and DSPP expression, respectively, then subset and embedded in UMAP space (FIGS. 2A and 1B). This analysis identified a heterogeneous cell population of six transcriptionally unique clusters including the dental papilla (DP), preodontoblast (POB), odontoblast (OB), subodontoblast (SOB), dental ectomesenchyme (DEM), and dental follicle (DF) (FIG. 2C) that were identified by putative marker genes (FIG. 1D).

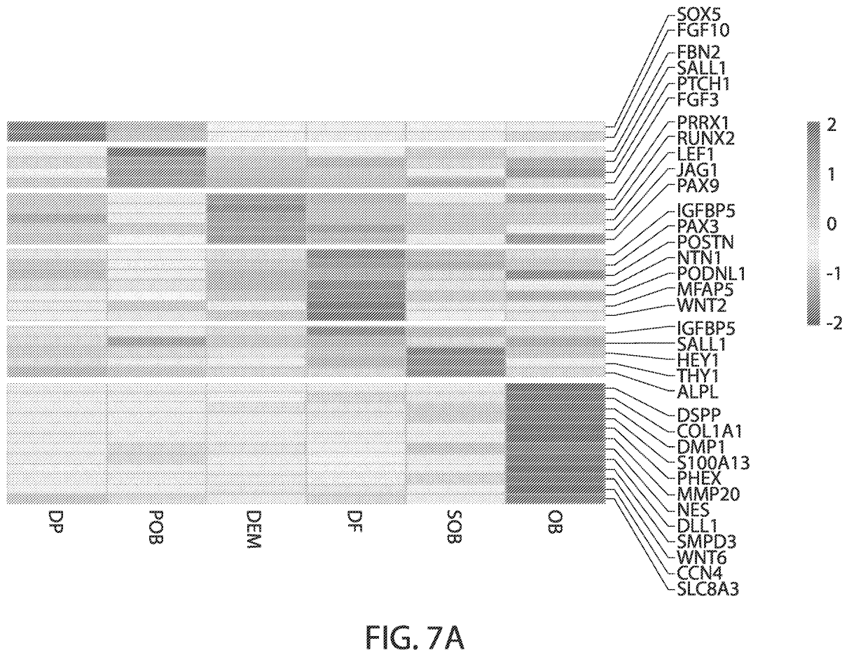

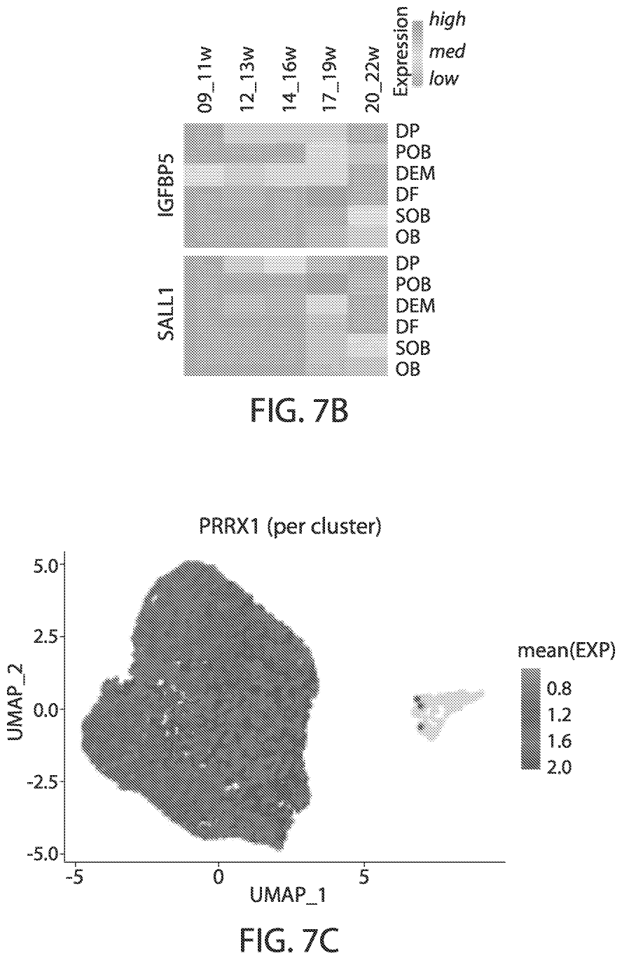

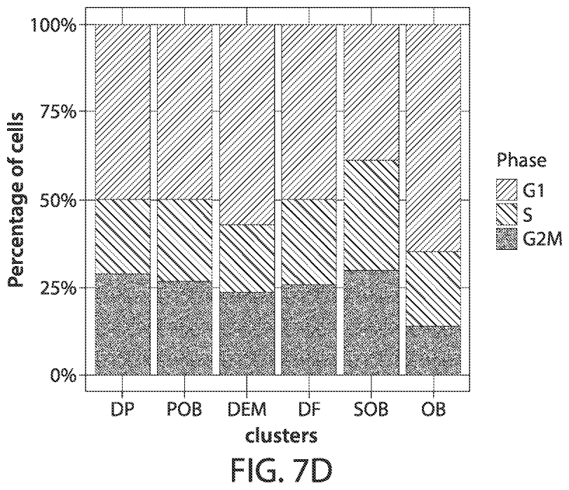

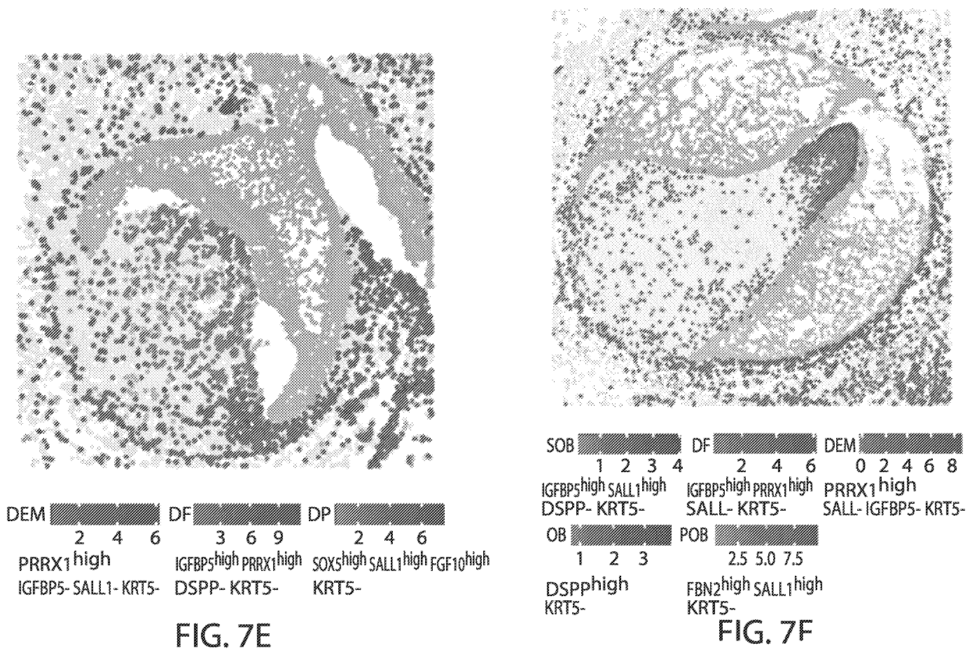

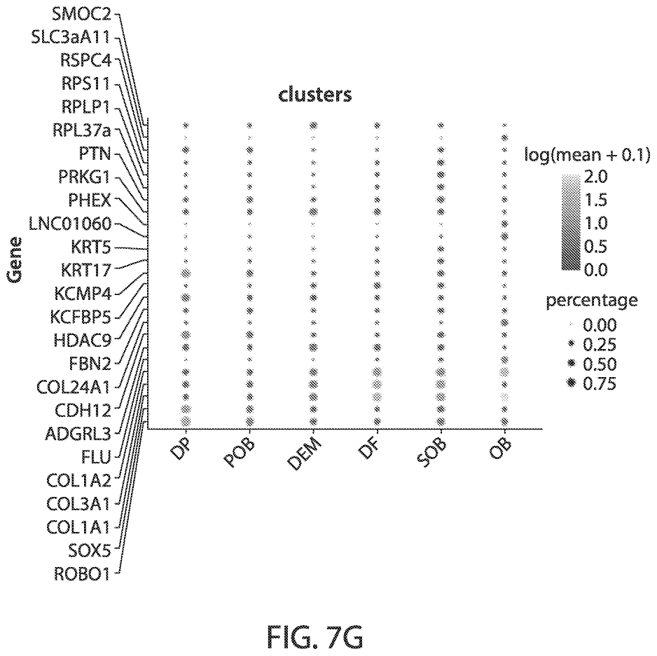

The DEM is identified by increased expression of neural crest derivative marker Paired Related Homeobox 1 (PRRX1)¹⁰⁷ and dental ectomesenchyme marker Runt-Related Transcription Factor 2 (RUNX2)⁷⁵. The DF shows high expression of Insulin Like Growth Factor Binding Protein 5 (IGFBP5)¹⁰⁸, as well as markers recently identified in the adult human dental follicle and periodontal ligament including Periodontal Ligament-Specific Periostin (POSTN), Netrin 1 (NTN1), Podocan Like 1 (PODNL1)^{53,109}, microfibrillar associated protein 5 (MFAP5), Wnt Family Member 2 (WNT2), and Paired Box 3 (PAX3)¹¹⁰. The DP has moderately high expression of PRRX1, an anticipated result as both the DEM and DP are putatively neural crest derived tissues, but is differentiated from the surrounding DEM by elevated expression of Spalt Like Transcription Factor 1 (SALL1)⁵³. DP is further uniquely identified by co-expression of SRY-Box Transcription Factor 5 (SOX5)¹¹¹ and Fibroblast Growth Factor 10 (FGF10)¹¹². POB cells show maintained elevated expression of SALL1, indicative of their heritage from the DP and previously observed in the mouse incisor⁵³, with significantly increased expression of Fibrillin 2 (FBN2)¹¹³. Similar to POB, SOB show moderate levels of SALL1 expression, suggesting a shared functional fate of POB and SOB to give rise to the OB. SOB are specified by high expression of IGFBP5¹⁰⁸ and markers previously observed in the SOB in mouse and rat molar respectively, including transcription factor Hairy And Enhancer Of Split-Related Protein 1 (HEY1)¹¹⁴, Thy-1 Cell Surface Antigen (THY 1)¹¹⁵, and Alkaline Phosphatase, Biomineralization Associated (ALPL)¹¹⁶. Finally, OB are identified by robustly known markers Dentin Sialophosphoprotein (DSPP)¹¹⁷, Dentin Matrix Acidic Phosphoprotein 1 (DMP1)¹¹⁶, and Collagen Type I Alpha 1 Chain (COL1A1)¹¹⁹⁻¹²⁰, as well as recently identified S100 Calcium Binding Protein A13 (S100A13)⁵³. (FIG. 7A, FIG. 7G)

To evaluate the function of each cluster, gene ontology analysis was performed using ViSEAGO, which uses data mining to establish semantic links between highly expressed genes in a given cluster. This analysis shows that DP and DEM are characterized by signaling, morphogenesis and adhesion, supporting their role as the precursor populations, while POB are characterized by their projection and proliferation, indicative of their precursor role and alignment to the edge of the dental pulp. SOB indicates activation, growth, and signaling, characteristics of a cell type sensing and influencing its environment, while OB shows GO-terms toward odontogenesis, tooth, and biomineral (FIG. 2B).

To assess progenitor sources and cell's progression towards differentiation, pseudotime trajectory analysis was performed. This analysis indicates two progenitor sources are present within the developing dental ectomesenchyme: the DP that give rise to POB and, subsequently, OB; and the DF that gives rise to SOB, which transition through a POB phase before giving rise to OB (FIG. 2C). Pseudotime analysis is supported by real-time density plots that show reduced progenitor type cell population density as the tooth germ develops, indicating fate commitment to OB lineage begins after 13 gw in human fetal development and is largely complete by 20 gw (FIG. 2D). Broad expression of dental ectomesenchyme marker PRRX1 is observed in both the DEM and DP (FIG. 7C), supporting previous findings³ that a shared cranial neural crest progenitor gives rise to both DP and DF. Thus, a simplified trajectory is proposed of both the odontoblast and dental follicle lineages FIG. 2E), with a shared PRRX1+ progenitor giving rise to both DEM and DP.

### III.III Developmental Trajectory Suggests SOB as a Novel Odontoblast Progenitor in Human Tooth Development.

Pseudotime trajectory indicates that two progenitor sources are present within the developing dental ectomesenchyme: the DP that gives rise to POB followed by OB; and the DEM that gives rise to the DF followed by SOB. SOB appear to transition through a POB state before giving rise to OB (FIG. 2C). While previous studies have shown that SOB, a spatial subgroup of regenerative mesenchymal cells, can give rise to odontoblast-like cells upon death of the primary odontoblasts⁵⁻⁶, their developmental and transcriptional identity remains uncharacterized. These results suggest that SOB lineage can differentiate to OB, not only to replace lost OB following injury as previously observed in animal models, but also during normal tooth development. Pseudotime trajectories are supported by real-time density plots (FIG. 2D), showing that OB progenitor DP population density decreases as the tooth germ develops, indicating fate commitment to OB lineage occurs prior to gw19 in human fetal development (FIG. 2J, 2K).

Cell cycle scoring provides important information on progenitor sources and developmental scoring. The analysis of the dental ectomesenchyme derived tissue supports DP and DEM as progenitor populations, as roughly 50% of cells are in G2M/S phase (FIG. 7D). It also suggests SOB as a progenitor source of OB during normal tooth development, as this cell type has the highest proportion of cells in G2M/S phase. As anticipated, OB shows low levels of G2M/S phase, indicating its role as a mature cell type that has terminally differentiated.

### III.IV Cell Types Identified by Sci-RNA-Seq are Present at Specific Spatio-Temporal Stages of Tooth Development In Vivo.

To validate the observed sci-RNA-seq DEG and novel biomarkers, RNAScope in situ multiplex hybridization⁴ was performed on human incisor and molar tooth germs in early (gw13) and late (gw19) stages of development (FIGS. 2F and 2H). This technique has proved incredibly useful in identifying cells that co-express multiple transcriptional markers.

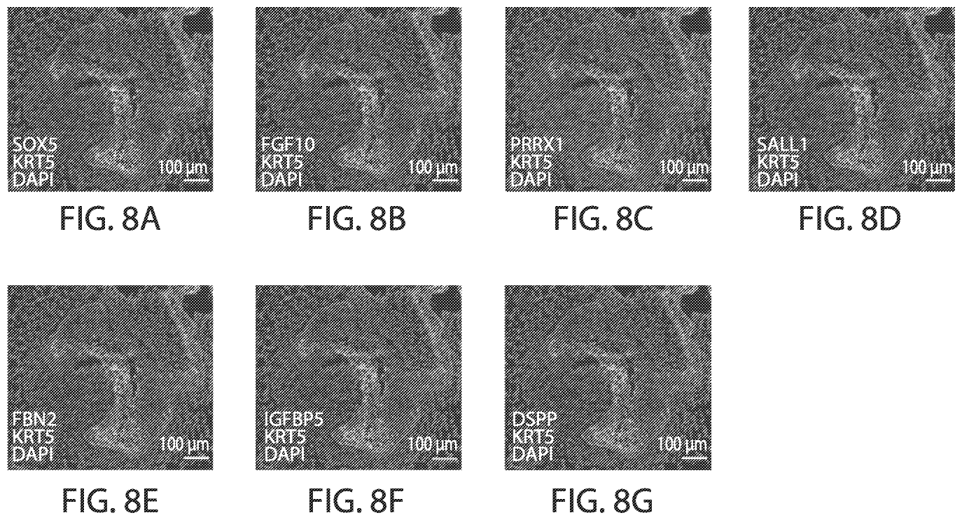

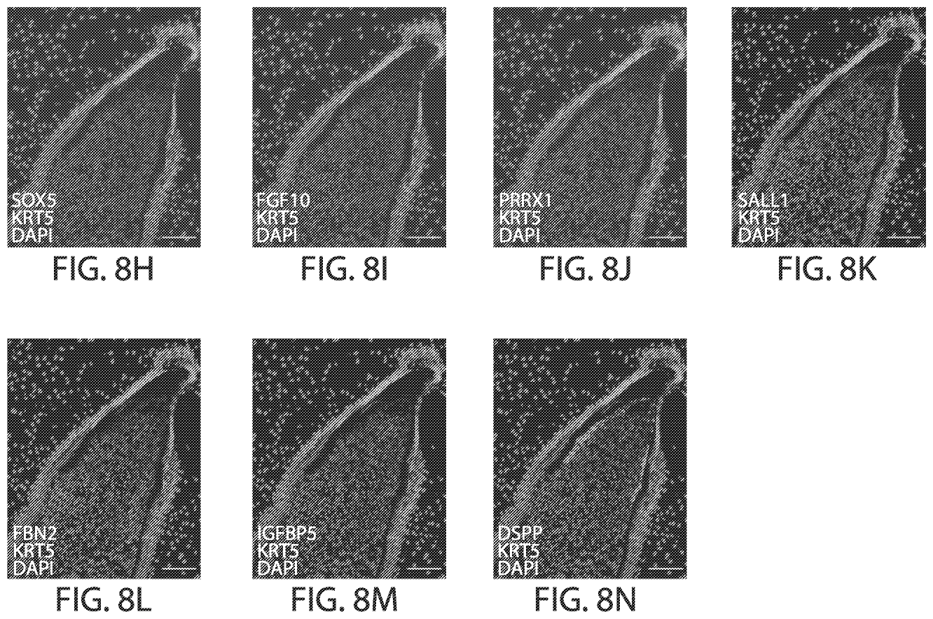

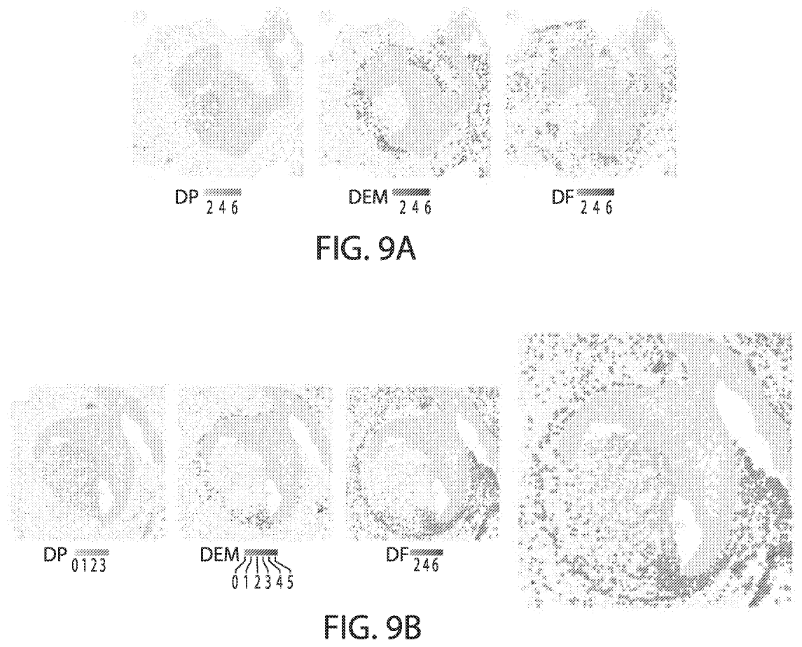

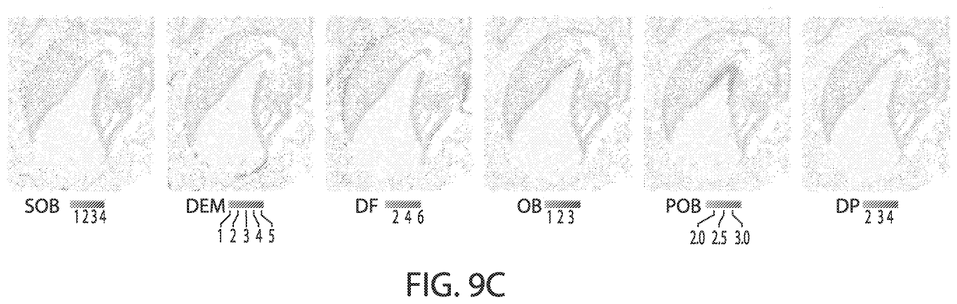

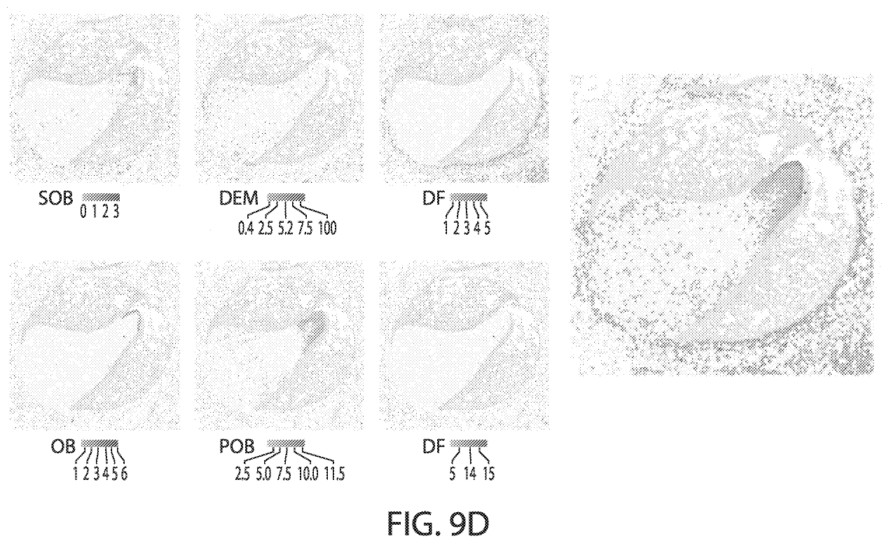

As predicted by computational analysis (FIG. 2A,2B), dental ectomesenchyme derived cell types display spatiotemporally specific expression patterns. At early tooth development (gw13), the dental pulp consists of SOX5/FGF10/SALL1+ dental papilla (DP) with PRRX1+ dental ectomesenchyme (DEM) localized to the apical portion (FIG. 2F, 2G, 2I; FIG. 7E; FIG. 8A-8G; FIG. 9A,9B). The entire tooth germ is surrounded by dental follicle (DF) cells, a pattern which persists to late tooth development (gw19). Interestingly, during early tooth development a small number of DF cells appear within the dental pulp (FIG. 2F,2G, 2J). By late tooth development (gw19) the dental pulp shows an organized hierarchy of cells at the incisal tip, with DSPP+ odontoblasts (OB) present at the incisal edge adjacent to FBN2+SALL1+ preodontoblasts (POB) aligned to the periphery of the dental pulp (FIG. 2H, 2I, 2K; FIG. 7F; FIG. 8H-8N; FIG. 9C, 9D). IGFBP5+SALL1+ subodontoblasts (SOB) are localized directly beneath the OB and, interestingly, intermingled with the POB at the pulpal periphery, suggesting SOB may transition through a POB-like state. The remaining dental pulp is a mixed population of DP, SOB, and POB with DEM localized to the apical region (FIG. 2K).

This analysis identifies a novel biomarker, IGFBP5, for the developing dental follicle (DF) (FIG. 7A,7B; FIG. 9A-9D). Further, novel human SOB biomarkers SALL1 and IGFBP5 were identified (FIG. 7A,7B; FIG. 8K,8M; FIG. 9C,9D), whose co-expression is spatiotemporally limited to define this cell type at gw19. This analysis reveals for the first time the presence of subodontoblast cells in the developing human tooth. Further, the maintained expression of IGFBP5 from early to late tooth germ development supports the hypothesis that SOB are DF derived cells, and that SOB fate commitment occurs prior to gw13.

Additionally, to assess expression of odontoblast markers at the protein level, immunohistochemical analysis was performed on human incisor tooth germs in early (gw15) and late (gw20) bell stage of development. As predicted by sci-RNA-seq, specific, spatio-temporal expression patterns were observed that differ markedly from early to late tooth development. Expression of OB marker DSPP and ameloblast (AM) markers AMBN and AMELX begins at gw20. Mirrored expression patterns of DSPP, AMBN, & AMELX between the OB & AM, were observed, supporting previous findings⁷² (FIG. 1F-1K; FIG. 3A-3Y).

To summarize, at gw13 the dental pulp consists of majority DP with DEM localized to the apical area, with the entire tooth germ surrounded by the DF. Interestingly, a small number of DF are present within the dental pulp, suggesting these cells have already committed to the SOB lineage at this early developmental stage. By gw19 the incisor pulp contains POB, SOB and OB with a smaller contribution of DEM at the apical foramen (FIG. 2J,2K). This suggests OBs are mainly derived from POB, while SOB serves as a reserve, with the capacity to differentiate to OB through a POB transitional state (FIG. 2C,2E). Further experiments will dissect how SOBs may play an inductive role in OB differentiation during tooth development.

### III.V Early FGF and BMP Activation, Followed by HH Signaling, are Critical to Human Odontoblast Development.

Analysis of the most active signaling pathways during human odontoblast differentiation predict that FGF, BMP, and HH signaling pathways are critical to directing this developmental trajectory. FGF and BMP (22%) are most active during DP to POB transition, with BMP activity dropping by roughly half (12%) during POB to OB transition and the majority of signaling derived from HH (22%) (FIG. 4A). During the transition from DP to POB, the dental epithelium derived cells are the major source of FGF and BMP signaling ligands including FGF1 and FGF23, which bind to FGFR1 possessing DP cells; and BMP10 that binds to BMPR2 and ACVR2A possessing DP cells, respectively. The dental ectomesenchyme was observed to be a smaller source of BMP ligands, including INHBC and GDF5, with a lesser contribution of autocrine signaling to the dental papilla (FIG. 4B,4D).

During the transition from POB to OB, the main source of BMP signaling ligand is predicted to be from the dental epithelium derived pre-ameloblast (PA) that secretes BMP10 and GDF9, which binds to BMPR2 and ACVR2A on the surface of POB. Interestingly, the subodontoblast (SOB) is the major source of BMP4 ligand, which binds to BMPR2 on the POB (FIG. 4C). HH signaling, the most active pathway in the transition, shows significantly increased DHH ligand expression in the PA, which is received by PTCH1 and HHIP possessing POB. FGF ligands are received from both dental epithelium and dental ectomesenchyme derived cells. NOTCH signaling, the third contributing pathway in the POB to OB transition, is largely activated by DLL4 secretion by the PA, received by NOTCH1, 2, and 4 possessing POB; and DLL1 secretion by the OB received by NOTCH1 on POB.

### III.VI Early FGF and BMP Activation, Followed by HH Activation, Leads to More Mature Odontoblast Development In Vitro.

To fully capture the developmental trajectory of the human odontoblast, human induced pluripotent stem cells (HiPSC) were first differentiated to a neural crest fate as described previously (FIG. 5A). Successful differentiation was confirmed by magnetic cell sorting for neural crest marker p75. 98% of differentiated cells positively expressed p75 (FIG. 5B). Differentiation was further validated by immunohistochemical analysis, which showed induced neural crest cells (iNC) express p75 and neural crest marker transcription factor AP-2α (FIG. 5C). Lastly, expression levels of neural crest markers PAX3 and SOX10¹²¹ were assessed at the transcriptional level via QPCR, which indicates significantly increased expression of both markers in iNC (FIG. 5D).

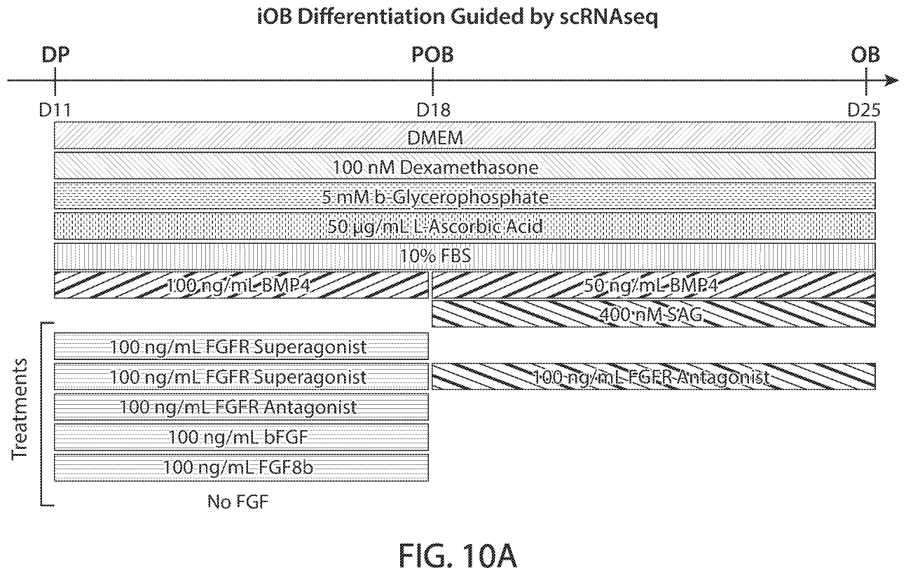

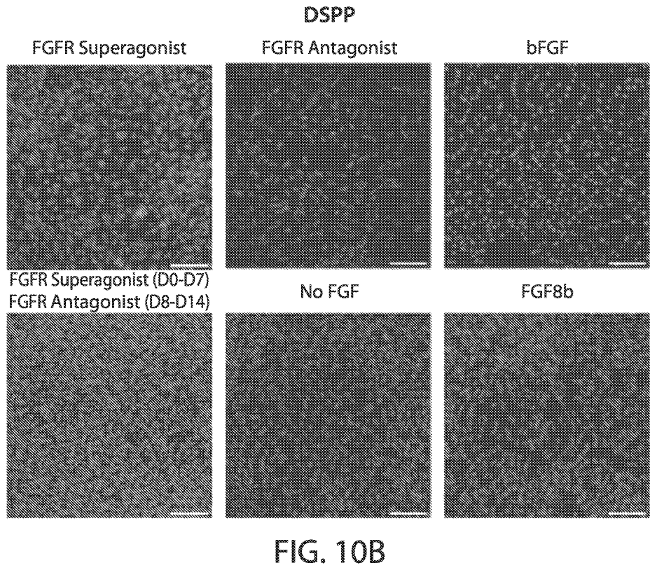

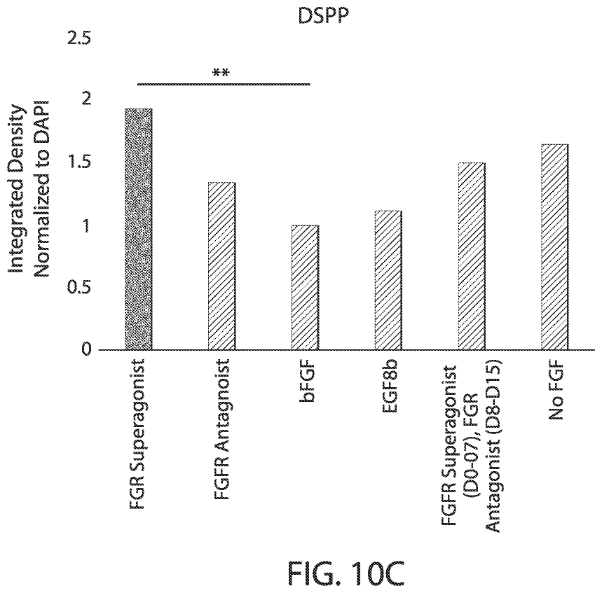

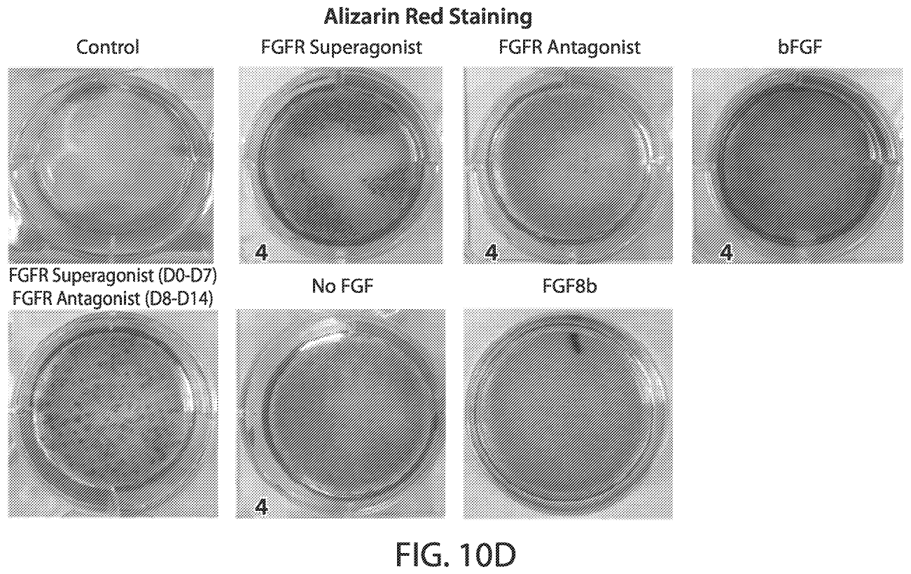

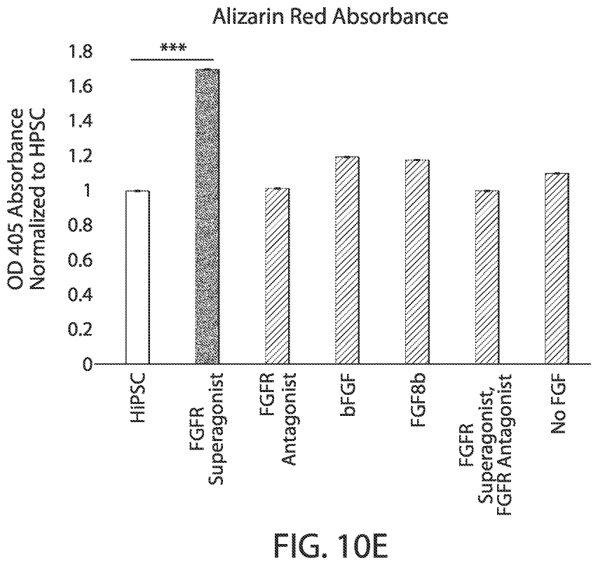

Next, iNC were biased to an odontoblast fate by activating the FGF, BMP and HH signaling pathways identified by computational analysis of sci-RNA-seq data (FIG. 4A and FIG. 5E) via supplementation with AI-designed FGFR minibinder superagonist, BMP ligand BMP4, and HH pathway agonist SAG, respectively (FIG. 10A). iNC derived odontoblast cells (iOB) have significantly decreased expression of neural crest markers SOX10 and PAX3 (FIG. 5F), and increased expression of odontoblast markers MSX1, DSPP and S100A13 compared to undifferentiated HiPSC (FIG. 5G). Further, iOB show increased DSPP expression at the protein level (FIG. 5H; FIG. 10B,10C) and enhanced mineralization capacity as assessed by Alizarin Red Staining (FIG. 5I; FIG. 10D,10E), indicating iOB have reached a mature state.

### III.VII Loss of DLX3 Arrests Odontoblast Maturity at the Preodontoblast Stage In Vitro.

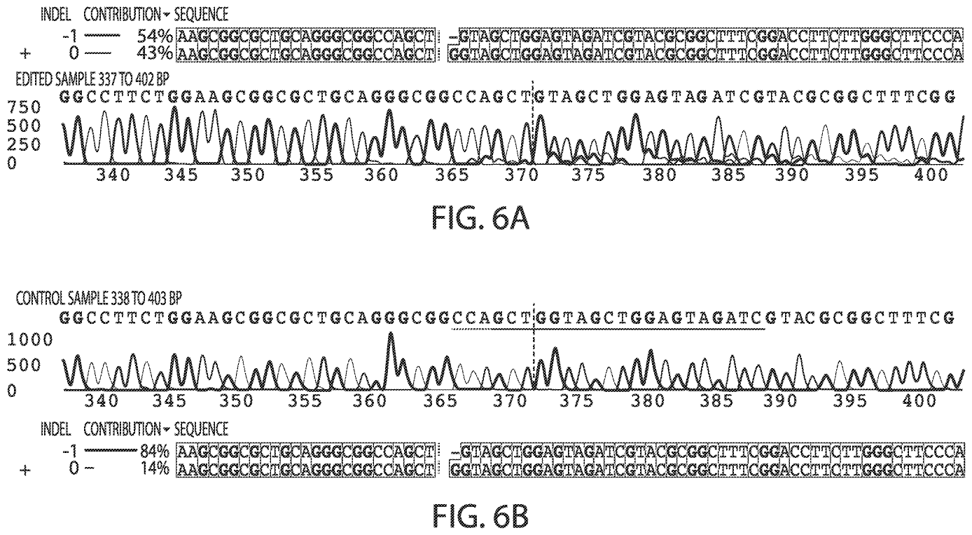

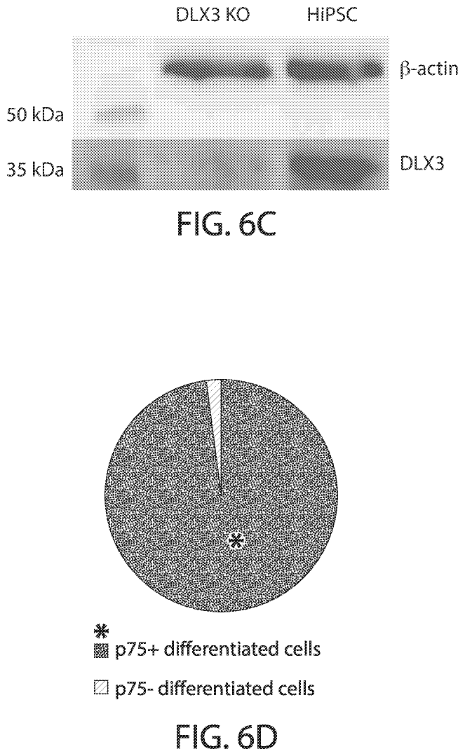

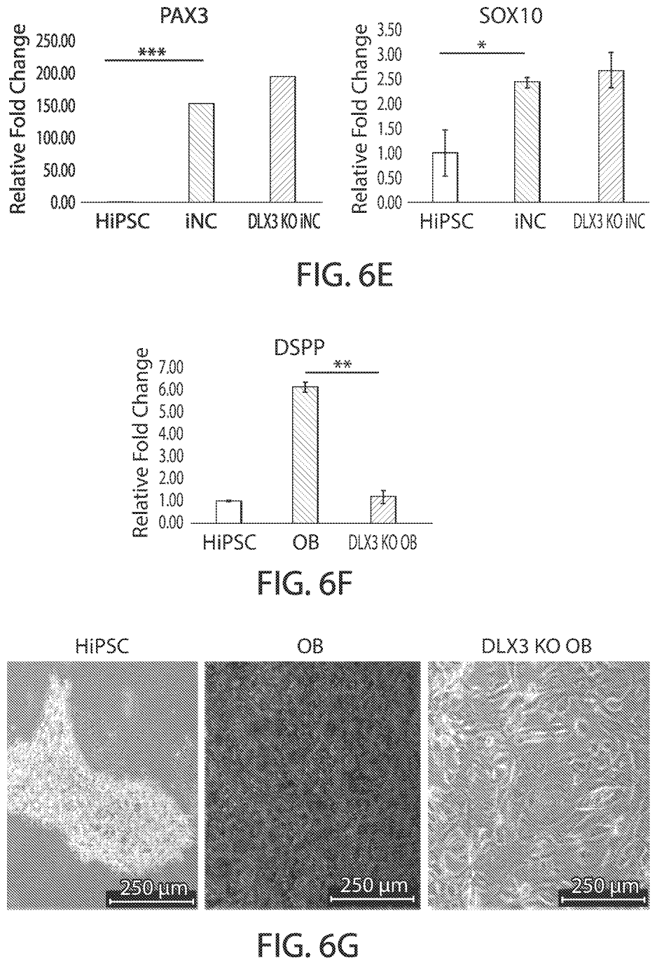

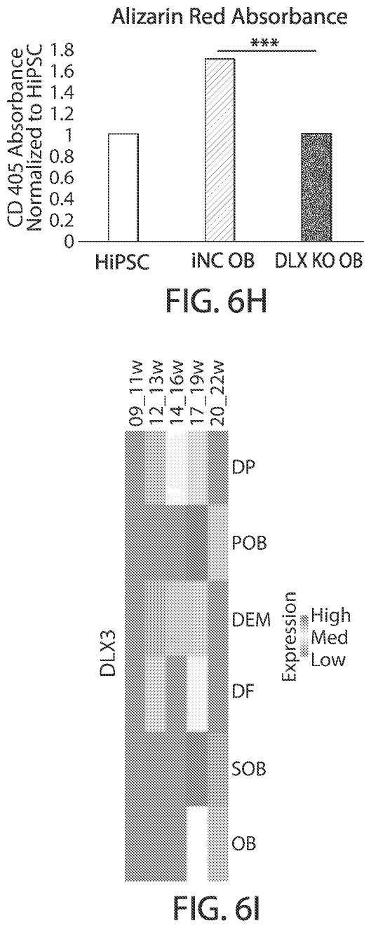

In order to assess the precise developmental stage at which DLX3 plays a role in human odontoblast development, a DLX3 knock-out line derived from iPSC was generated and differentiated it towards neural crest followed by odontoblast fate as described above. Presence of deletion mutation within exon 2 of the DLX3 gene was confirmed by Sanger sequencing via Genewiz, illustrating that 54% of cells possess a single base pair deletion (indel mutation) at site 374 with removal of a single glycine nucleotide (FIG. 6A). It appears that the DLX3 mutant is dominant in a mixed population, as sequencing of DLX3 KO following iNC differentiation shows a population shift to 84% of cells possessing the indel mutation (FIG. 6B). Loss of DLX3 protein was confirmed by Western blot analysis, which showed no band formation validating loss of DLX3 protein (FIG. 6C).

Differentiation of DLX3 mutants to neural crest fate appears unaffected by the loss of DLX3, illustrated by no significant changes in percent of p75+ cells produced via magnetic cell sorting (FIG. 6D) or neural crest markers PAX3 or SOX10 expression (FIG. 6E). Mutants were then further differentiated toward odontoblast fate as described previously. Interestingly, loss of DLX3 results in significantly decreased DSPP expression (FIG. 6F), indicating DLX3 plays a critical role in DSPP expression. Further, mutant iOB show significantly inhibited mineralization capacity via Alizarin Red Staining (FIG. 6G-6H). In the human dental pulp, DLX3 is highly expressed in the POB (FIG. 6I), indicating a critical role for this transcription factor in OB development and supporting the hypothesis that loss of DLX3 prevents the transition from POB to mature OB through decreased expression of DSPP.

### Discussion & Conclusions

Odontoblasts are required for formation of the tooth's dentin, which composes the majority of the tooth's mineralized tissue. Dentin provides the tooth's toughness, or resistance to crack propagation, and tensile strength, or distribution of biomechanical forces to the surrounding periodontium. While odontoblasts persist throughout life, their number and ability to produce dentin significantly decreases with age. Human dental pulp and follicle cell types were identified that significantly and precisely promote the differentiation of odontoblasts. Importantly, the subodontoblast was identified as a novel odontoblast progenitor in human tooth development. Analyzing the signaling interaction in odontoblast differentiation allowed for predicting the signaling molecules that are needed to recapitulate odontoblast development in vitro. Utilizing these findings, a novel differentiation protocol was developed to drive the differentiation of iPSCs toward odontoblast (iOB) that expresses mature odontoblast markers and secretes mineralized tissue. Finally, this information was used to dissect the role of dentin defect associated DLX3 in odontoblast development, identifying the precise molecular step at which odontoblast differentiation is arrested with loss of this critical transcription factor.

Single cell analysis of the developing dental pulp and follicle identified a group of six transcriptionally unique cell types of dental ectomesenchymal lineage. This analysis identifies novel biomarkers for the dental ectomesenchyme derived cell types in the developing human tooth germ that give rise to mature odontoblasts, characterizing each cell type with a specific transcriptional signature.

The dental papilla (DP) is identified by co-expression of signaling molecule FGF10 and transcription factors SOX5 and SALL1. FGF10 is a growth factor that plays critical roles in cell proliferation, differentiation and migration¹⁰⁴. In the dental pulp, FGF10 is expressed in the mouse molar DP early in tooth development with decreased expression in the odontoblast, suggesting a role in odontoblast maturation¹¹². SOX5 has previously been observed in the mouse molar dental ectomesenchyme at early tooth developmental stages¹¹¹ and is known in other tissues to play regulatory roles in both BMP¹²³ and HH¹²⁴ signaling pathways. Crosstalk between the FGF signaling pathway and SOX transcription factors has been observed to impact osteoblast development¹²⁵, however, their interaction in odontoblast development remains unknown. SALL1 has previously been observed in the mouse incisor preodontoblast (POB)⁵³, indicating synchrony of odontoblast progenitor gene expression patterns between murine and human models. Elevated expression of SALL1 in the DP-expression that is maintained in the human preodontoblast, subodontoblast, and odontoblast cells-supports the hypothesis that odontoblast fate commitment occurs prior to odontoblast orientation at the periphery of the pulp.

The developing human preodontoblast (POB) shows a transcriptional signature of increased FBN2 expression and maintained SALL1 expression. Fibrillins are a major component of microfibrils and elastic fibers, structures critical for cellular mechanical stability and regulating cell development by sequestering TGF-β and BMP signaling molecules¹²⁶. FBN2 has been shown to increase in the periodontal ligament in response to mechanical stress¹²⁷. Increased FBN2 expression in the POB supports the dogma that these precursor cells migrate first towards the pulp periphery, then the incisal edge, before giving rise to OB. Clarifying the role of FBN2 in controlling signaling interactions of the differentiating POB will require further study.

The findings identify the subodontoblast (SOB) in the developing human tooth germ for the first time, and suggest this cell type as a novel odontoblast progenitor. The SOB is capable of giving rise to odontoblasts during injury repair; in addition to this reparative role, the studies suggest SOB can give rise to OB not only following loss of the primary OB, as previously reported, but during normal healthy human tooth development. This hypothesis is supported by computational analysis of sci-RNA-seq data of the developing tooth germ, indicating 1) shared expression of biomarker SALL1 in dental papilla (DP) progenitor that is maintained in both preodontoblast (POB) and SOB cells at later developmental stages; 2) pseudotime analysis (Monocle3) indicating that SOB transition through a POB state before giving rise to OB; and 3) cell cycle scoring shows 60% of SOBs are actively cycling through G2M/S phases, supporting their role as a source of OB progenitor. Further, In situ visualization of SOB finds these cells spatially localized to the subodontoblastic region directly beneath the OB and intermingled with POBs at the periphery of the dental pulp, strengthening the suggestion SOB cells transition through a POB-like state prior to differentiating to OB during normal tooth development.

IGFBPs bind with high affinity to IGF signaling ligands, inhibiting their interactions with IGF receptors¹²⁸. IGFBP5, a highly conserved protein in vertebrate organisms, has previously been shown to regulate cell migration, proliferation, and survival¹²⁹⁻¹³⁰. IGFBP5 has been observed in the dental papilla and odontoblasts of mouse incisor and is proposed to play a role in differentiated odontoblasts cell survival and maintenance¹⁰⁸. The observation of localized IGFBP5 expression in the SOB supports the suggested role of IGFBP5 in maintaining a progenitor population that is actively differentiating towards a more mature OB fate. Further studies using lineage tracing methods are needed to verify this exciting hypothesis, revealing if SOBs encompass an inductive role in OB differentiation during human tooth development.

For the first time in human development, the studies have revealed in extreme detail, the signaling pathways that govern each transition between odontoblast cell identity. Previous studies of hypodontia and tooth agenesis have shown that disruption of WNT, BMP, and FGF signals result in defective tooth development. However, the detail with which the study has revealed the role of these pathways at various points in odontoblast development may more mechanistically explain how defects in these pathways lead to tooth loss or tooth agenesis. This study reveals at a molecular level the specific effectors that control human odontoblast development, indicating that FGF, BMP, and HH are the critical signaling pathways contributing to odontoblast differentiation. This knowledge can be used to develop therapeutic agents to induce dentinogenesis clinically and was applied here to develop an efficient HiPSC-derived odontoblast differentiation protocol (iOB).

The single cell-based differentiation protocol of iPSC to odontoblast (iOB) produced mature odontoblast cells that showed significantly increased expression of odontoblast markers and enhanced mineralization capacity. While odontoblasts persist throughout life and are able to respond to injury by secreting tertiary dentin, their number and ability to produce dentin significantly decreases with age, posing a challenge to regenerative dentistry. If the primary odontoblasts are lost, pulp-derived mesenchymal cells are induced to differentiate into odontoblast-like cells, forming reparative dentin.

DPSC have previously been shown to successfully differentiate towards osteogenic and odontogenic fates²³⁻²⁵ and have been characterized in detail²⁶. Unfortunately, DPSC expansion and regeneration capacity is limited²⁷, showing a dramatic decrease in regenerative capacity with increased age²⁸. Thus, the iOB protocol described here provides a model essential to study genetic diseases affecting dentin formation, interlayer communication involved in odontogenesis, as well as regeneration of tooth dentin structure.

Odontoblasts are believed to develop through reciprocal, repeated signaling interactions with the dental epithelium derived ameloblasts. The signaling pathway analysis indicates that the majority of signaling ligands critical for odontoblast development are produced by the dental epithelium derived inner enamel epithelium and pre-ameloblast at early and late tooth development, respectively. Interestingly, the bulk of BMP signaling ligands received by the POB as it transitions to OB are secreted by the SOB, indicating a supportive role for this novel cell type in human OB development. While previous studies have focused on the role of a single signaling pathway, many others have highlighted the importance of crosstalk between pathways in tooth development and maintenance¹³¹⁻¹³³. The predictive pathway analysis highlights not only the primary pathway responsible for each stage, but ranks the other pathways involved, meaning that the study will facilitate the investigation into both previously identified and yet undescribed crosstalks in driving forward development. The detailed analysis provided in this study will facilitate more detailed and informed studies on degenerative dental diseases and can lead to the development of more effective ways to mitigate or reverse tooth loss. Furthermore, the work with AI-designed, de novo receptor mini-binders that specifically bind and either activate or inhibit target receptor signaling^{134,135} now reveals a novel, highly simplified method to identify the exact stage-specific signaling pathway required in the differentiation process. The method described in this study using the de novo FGFR-minibinder to unravel the FGFR pathway requirement in odontoblast maturation will be generally applicable and specific to any signaling pathway analyzed in differentiation of normal and disease organoids.

Studies focused on co-culture of the iOB described here with the lab's HiPSC-derived ameloblasts (iAM)¹³⁶ will provide an unprecedented tool for studying the signaling patterns exchanged between these tissue types during tooth development, likely resulting in further maturation of both cell types.

Though DLX3 has been shown to be expressed in murine neural crest cells, the study illustrates this transcription factor is not required for differentiation of human induced pluripotent stem cells to neural crest fate. The findings support previous murine models indicating loss of DLX3 results in downregulated expression of odontoblast marker DSPP. Further, it has been shown that DLX3 mutants have inhibited mineralization capacity, indicating odontoblast maturity is arrested by loss of this transcription factor. Importantly, the findings indicate that loss of DLX3 impacts odontoblast development at the POB stage, deepening the understanding of DLX3's role in human odontoblast development and taking the field one step closer to developing therapies for Tricho-Dento-Osseous Syndrome.

The overall significance of this study is threefold: 1) providing unprecedented insight at the single cell level into cell types of the developing tooth dental ectomesenchyme; 2) applying the revealed molecular signaling that controls human odontoblast cell lineage commitment during differentiation to generate a HiPSC-derived odontoblast differentiation method (iOB); 3) utilizing the iOB tool to study the molecular mechanism of human odontoblast differentiation in states of health and disease in order to design appropriate therapies.

Incorporated by reference, with variations and pseudogenes:

NCBI Gene Expression Omnibus under accession number (GSE184749). The mouse incisor dataset used for comparison can be downloaded from the accession code GSE146123.

### Example 2

Single Cell RNA Sequencing Reveals Human Tooth Type Identity and Guides In Vitro hiPSC Derived Odontoblast (iOB).

Over 90% of the U.S. adult population suffers from tooth structure loss due to caries. Most of the mineralized tooth structure is composed of dentin, a material produced and mineralized by ectomesenchyme derived cells known as odontoblasts. Clinicians, scientists, and the general public share the desire to regenerate this missing tooth structure. To bioengineer missing dentin, increased understanding of human tooth development is required. It was interrogated at the single cell level, the signaling interactions that guide human odontoblast and ameloblast development and which determine incisor or molar tooth germ type identity. During human odontoblast development, computational analysis predicts that early FGF and BMP activation followed by later HH signaling is crucial. Application of this sci-RNA-seq analysis generates a differentiation protocol to produce mature hiPSC derived odontoblasts in vitro (iOB). Further, the critical role of FGF signaling was elucidated in odontoblast maturation and biomineralization capacity using the de novo designed FGFR1/2c isoform mini binder scaffold C6. Using computational tools, it was shown on a molecular level how human molar development is delayed compared to incisors. It was revealed that enamel knot development is guided by FGF and WNT in incisors and BMP and ROBO in the molars, and that incisor and molar ameloblast development is guided by FGF, EGF and BMP signaling, with tooth type specific intensity of signaling interactions. Dental ectomesenchyme derived cells are the primary source of signaling ligands responsible for both enamel knot and ameloblast development.

### Introduction

Untreated dental caries is the most prevalent disease globally, with the Center for Disease Control finding that 90% of adults in the United States (U.S.) suffer from dental caries (1). Further, dental pulp disease was the primary diagnosis for over 400,000 emergency department visits in the U.S. (2), highlighting the need for significant resources to restore both the dental pulp and the mineralized dentin tooth structure it produces. The current method to return form and function to the lost tooth structure with artificial prosthesis such as fillings and crowns can initiate a continuous cycle of restoration replacement, each replacement leading to increased tooth structure loss due to preparation requirements, recurrent caries, or fracture (3). This process, known clinically as the “tooth cycle of death”, can ultimately lead to tooth removal and replacement with a dental implant, currently one of the best tooth alternatives. Importantly, after 9 years, 45% of dental implants develop peri-implantitis (4), an inflammatory process that can lead to loss of the implant and surrounding bone. At this stage the patient often suffers from insufficient bone levels to support a new dental implant, leaving both the patient and clinician in a treatment quandary. Regenerative dentistry seeks to produce stem cell tools to regenerate missing tooth structure. The need for a tooth organoid is paramount.

Reciprocal and continual signaling interactions between the cells of the dental ectomesenchyme and dental epithelium are required for tooth formation (5), disruption of which arrests tooth development (6). Multiple signaling pathways are active throughout tooth development including fibroblast growth factor (FGF), bone morphogenic protein (BMP), hedgehog (HH), and wingless/integrated (WNT) (7,8). Recently, single cell analysis of the developing human oral cavity revealed that transforming growth factor beta (TGF-β), neurotrophin (NT), HH, BMP, and WNT play critical roles in ameloblast development (9). However, the specific cells of the dental pulp involved in these signaling interactions and their impact on ameloblast development remains unknown.

The majority of mineralized tooth structure is composed of dentin, a vital tissue produced and mineralized by odontoblasts (OB), overlaid by a coat of enamel synthesized by ameloblasts. Murine OB development has been characterized in detail (10). However, mice constantly replenish missing tooth structure through several stem cell niches absent in human teeth, posing translational challenges between the species. Human OB differentiation and maturation remains largely unknown due to the rarity of fetal tissue samples. Importantly, the recent single cell sequencing of the developing human OB lineage (9) now licenses a deeper understanding of OB differentiation towards regenerative dentistry.

Beyond guiding cellular lineage commitment and differentiation, intercellular signaling also shapes the type of tooth that is formed (e.g. incisor or molar). At the early bud stage, odontogenic potential shifts from the overlying dental epithelium to the neural crest derived dental ectomesenchyme (11). In mice, it has been shown that determination of tooth identity is regulated by the dental ectomesenchyme derived cells (6). The dental epithelium derived enamel knot acts as a signaling center that triggers cell proliferation and cytodifferentiation of the dental papilla during tooth morphogenesis (12). Importantly, the lab recently identified FGF4 as a biomarker for the human enamel knot (9). Yet in humans the dental cell types and the intercellular signaling patterns that shape crown morphology, and therefore tooth type, remains unknown.

It was previously revealed a single cell combinatorial indexing RNA sequencing (sci-RNA-seq) atlas of developing human tooth germ, identifying the dental ectomesenchyme and dental epithelium derived cell types comprehensively (9). The signaling interactions were interrogated at the single cell level that guide human odontoblast and ameloblast development and that determine incisor or molar tooth germ type identity. The sci-RNA-seq predicted signaling pathways were applied to generate a hiPSC derived odontoblast differentiation method (iOB) using de novo designed FGFR1/2c isoform mini binders to produce a tool for regenerative dentistry therapeutics and disease modeling goals.

### Materials and Methods

### Single Cell RNA Sequencing of Human Fetal Tooth Germs.

### Single Cell Combinatorial Indexing RNA Sequencing.

As described previously (9), 312 human fetal toothgerm samples (201 incisors and 111 molars) and 22 fetal jaw segments (10 anterior and 12 posterior) were collected from five gestational week (gw) groups representing the following developmental stages for deciduous tooth differentiation: the bud stage (9-11 gw), cap stage (12-13 gw), early bell stage (14-16 gw), and late bell stage (17-22 gw) (7,13). Due to the limited number of samples obtained for 14-16 gw, molar and incisor tissues were combined prior to single cell RNA sequencing. Single cell combinatorial indexing RNA sequencing (sci-RNA-seq) was performed in collaboration with the Brotman Baty Institute (14). Low quality reads were removed by filtering UMI reads higher than 1500 and lower than 100, followed by removal of all mitochondrial reads. Utilizing the Monocle 3 workflow (15,16) data underwent normalization by size factor, preprocessing, dimension reduction into Uniform Manifold Approximation and Projection (UMAP) space (17), and unsupervised clustering producing grouping of the cells into clusters based on similarity of gene expression (14). Cell cycle effect was regressed out (18) by simple linear regression using Seurat (19). Cell lineage trajectories and pseudotime were produced using Monocle 3. Pseudotime is calculated from dynamic changes in differentially expressed genes (DEG) and defines a cell's progress along a developmental trajectory (15).

### Identification of Critical Signaling Pathways Via the Top Pathway Analysis.

The lab has developed a comprehensive analysis pipeline to evaluate signaling pathway activity based on ligand-receptor interactions and downstream activity (9). Prior to analysis, a differentiation trajectory with known progenitors, maturely differentiated target cells, and neighboring support cells present at the same developmental stage must be defined. This pipeline utilizes the talklr package (20) to identify and rank incoming ligand signals to the progenitor cell of interest, filtering for ligand-receptor interactions associated with major signaling pathways of interest. Each interaction is assigned a normalized interaction score, which is calculated by dividing the sum of interaction scores across all pairwise cell-cell interactions. The DEsingle package (21) was utilized to produce the DEG between the progenitor and maturely differentiated target cells (False Discovery Rate <0.1 and Fold-Change >2). The scMLnet package (22) was used to generate a multilayer network modeling the upstream ligand-receptor pairs from talklr, downstream transcription factors (TF), and their target genes from DESingle. Connectivity of each layer of the model was scored to predict which pathway is the most active. Scores were calculated by determining target gene fold-change; mean TF-target genes associated with a given TF; sum of TFs associated with a given receptor; sum of receptors associated with a given ligand; and finally sum of ligands that are associated with a given signaling pathway. Score normalization is performed at each layer. Finally, the pipeline ranks signaling pathways by activity score, indicating the most active pathways including the key drivers of differentiation between progenitor and target maturely differentiated cells.

### Code Availability.

The custom R codes used in this manuscript are available in github.com/Ruohola-Baker-lab/Tooth_sciRNAseq.

Human Induced Pluripotent Stem Cell Derived Odontoblast Differentiation Guided by Sci-RNA-Seq (iOB).

### Human Induced Pluripotent Stem Cell Culture.

Human induced pluripotent stem cell (hiPSC) line WTC-11 (Coriell GM25256) (23,24) were seeded on 6-well plates and cultured in mTeSR stem cell medium (StemCell Technologies 85850) with daily media changes until cells reach ˜70% confluency (25, 26). Cells were passaged using Accutase (Sigma-Aldrich A6964). ROCK inhibitor (ROCKi) (Stemcell Technologies) is added to mTeSR for initial 24-hours. To maintain hiPSCs and prevent fusing or premature differentiation of colonies, regular observation under low-power microscopy were performed with colony passaging as necessary.

hiPSC Derived Neural Crest Differentiation (iNC).

This project applies the protocol previously described to produce iPSC derived neural crest (iNC) through dual SMAD inhibition and early WNT activation (27-29). hiPSC are seeded at 32,000 cells per well on 6-well matrigel coated plates and maintained in mTeSR until 70% confluent. Differentiation is induced with addition of Basal Neural Maintenance Media (BNMM), which consists of 250 mL DMEM/F12+glutamine (Gibco 11320-033) and 250 mL neurobasal media (Gibco 21103-049) supplemented with 2.5 mL N2 (Gibco 17502-048), 5 mL B27 (Gibco 17504-044), 2.5 mL GlutaMax (Gibco 35050-061), 2.5 mL ITS-A (Gibco 51300-044), 400 μL 2-Mercaptoethanol (Thermo Fisher Scientific 21985023), and 2.5 mL NEAA (Thermo Fisher Scientific 11140050). On Day 0, BNMM is supplemented with 10 μM SB 431542 (Biogems BG6675SKU301) and 1 μM LDN 193189 (Biogems BG5537SKU106) for dual SMAD inhibition; inhibition is maintained until Day 4 and Day 3 respectively. On Day 2, WNT is activated via supplementation with 3 μM CHIR 99021 (Tocris Bioscience 4423), which is maintained until Day 11. Media change occurred daily.

Magnetic Cell Sorting For p75+ iNC.

On Day 11, cells were lifted via Accutase (Sigma-Aldrich A6964) and resuspended in an IMAG buffer consisting of 0.5% bovine serum albumin and 2 mM EDTA (Invitrogen 15575-038). iNC were isolated with addition of PE-conjugated p75 (also known as Nerve Growth Factor Receptor, NGFR, and CD271) antibody (Thermo Fisher Scientific 12-9400-42) and Anti-R-PE magnetic beads (BioSciences 557899). p75+ cells were eluted, resuspended in media, and plated on 24-well matrigel coated plates at a density of 250,000 cells per well.

### De Novo Designed FGFR1/2c Isoform Mini Binder Expression.

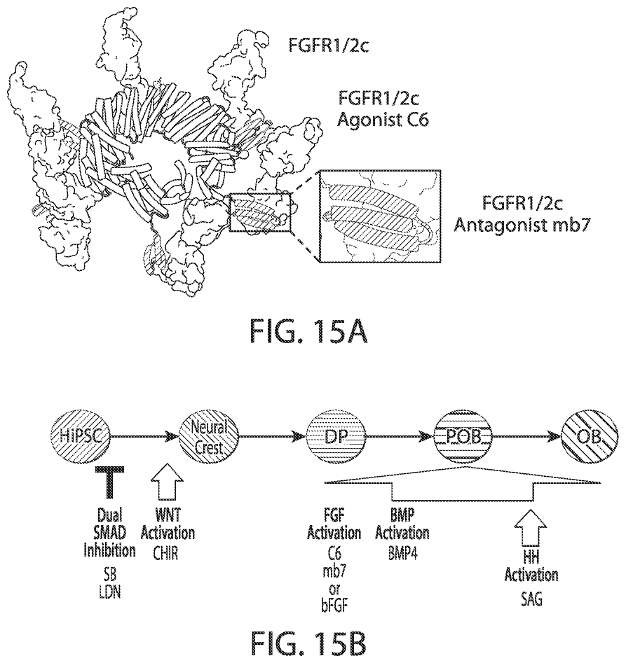

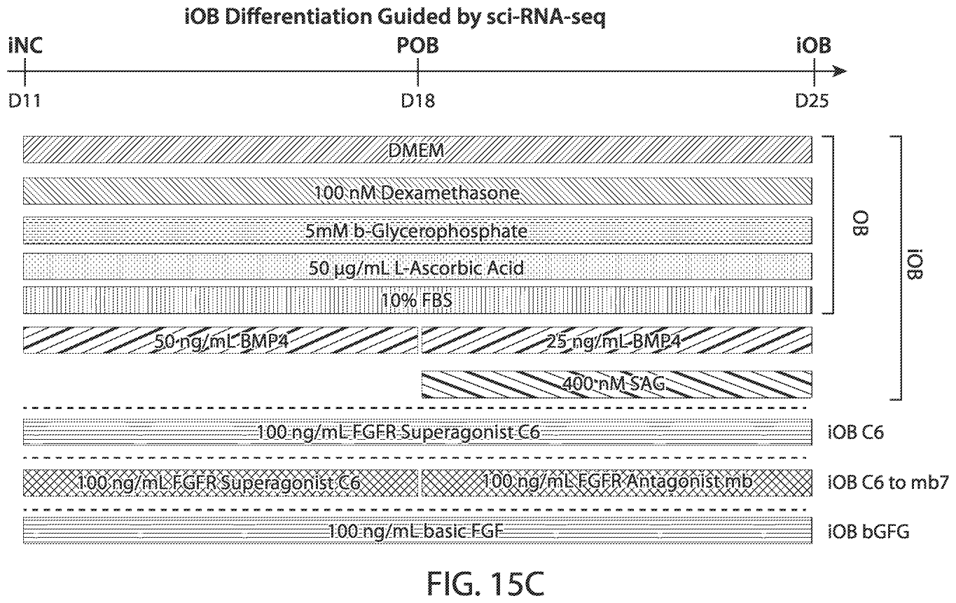

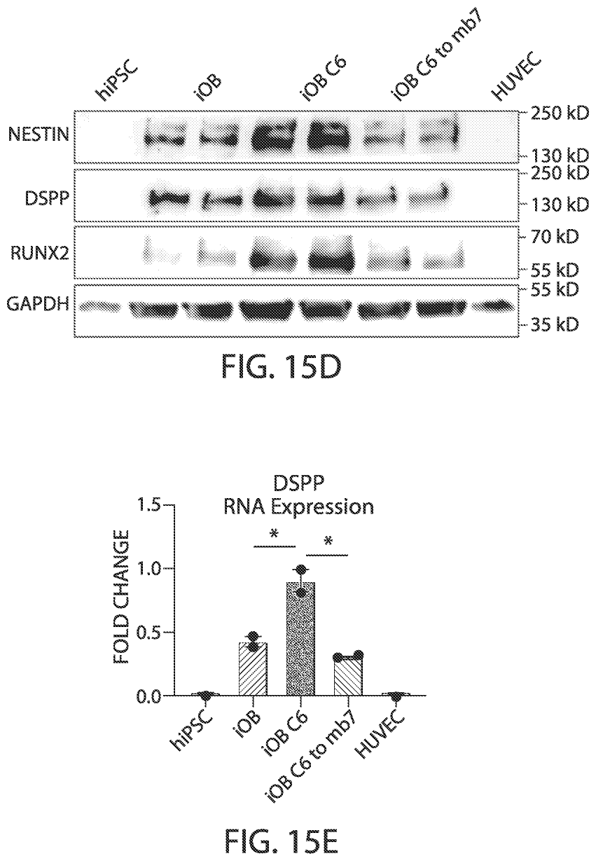

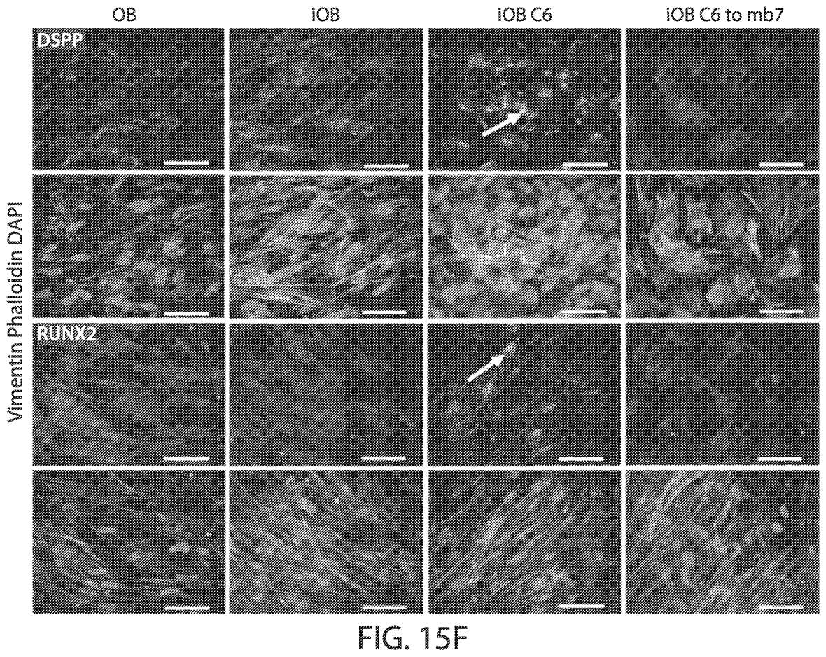

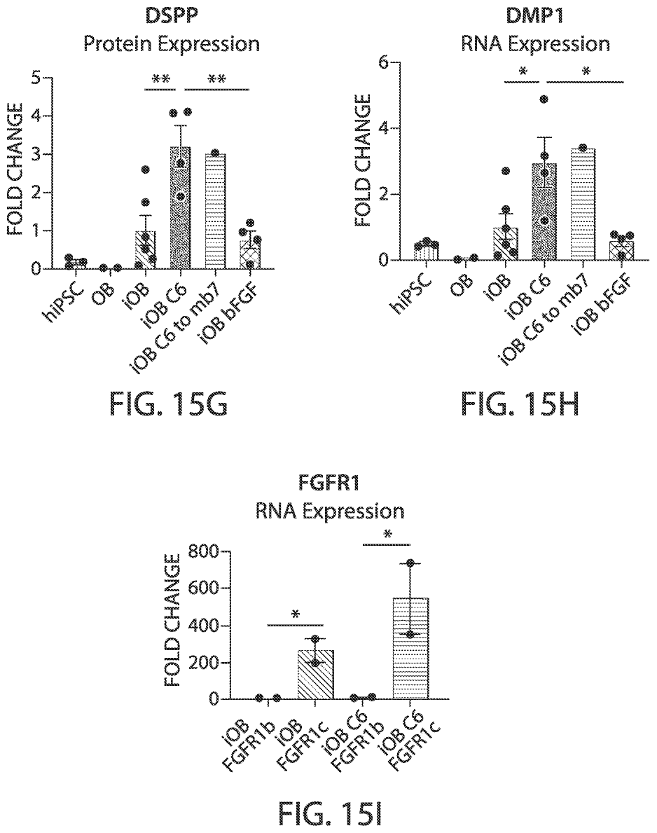

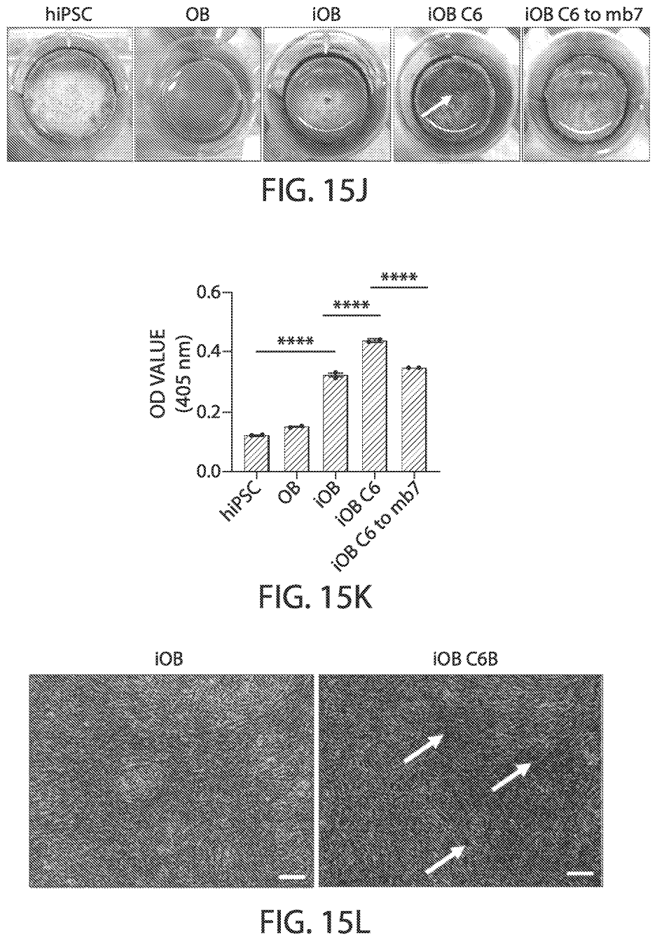

The sequences encoding the de novo designed fibroblast growth factor receptor-c (FGFR1/2c) isoform mini binder alone (hereby referred to as mb7) or fused to a hexameric scaffold (hereby referred to as C6) (FIG. 15A) were synthesized and cloned into modified pET-29b(+) E. coli plasmid expression vectors (GenScript, N-terminal 8-His tag followed by a TEV cleavage site). The sequence of the N-terminal tag is MSHHHHHHHHSENLYFQSGGG (SEQ ID NO: 8), which is followed immediately by the sequence of the designed protein (31,32). Plasmids were transformed into chemically competent E. coli Lemo21 cells (NEB). The protein expression was performed using Studier autoinduction medium supplemented with antibiotic, and cultures were grown overnight. Then, IPTG was added to a final concentration of 500 mM and the cells were grown overnight at 22° C. for expression. The cells were collected by spinning at 4,000 g for 10 min and then resuspended in lysis buffer (300 mM NaCl, 30 mM Tris-HCL (pH 8.0), with 0.25% CHAPS for cell assay samples) with DNase and protease inhibitor tablets. The cells were lysed with a sonicator (Qsonica Sonicators) for 4 min in total (2 min each time, 10 s on, 10 s off) with an amplitude of 80%. The soluble fraction was clarified by centrifugation at 20,000 g for 30 min. The soluble fraction was purified by immobilized metal affinity chromatography (Qiagen) followed by FPLC SEC (Superdex 75 10/300 GL, GE Healthcare). The protein samples were characterized by SDS-PAGE, and purity was greater than 95%. Protein concentrations were determined by absorbance at 280 nm measured with a NanoDrop spectrophotometer (Thermo Fisher Scientific) using predicted extinction coefficients.

### iNC Derived Odontoblast Differentiation.

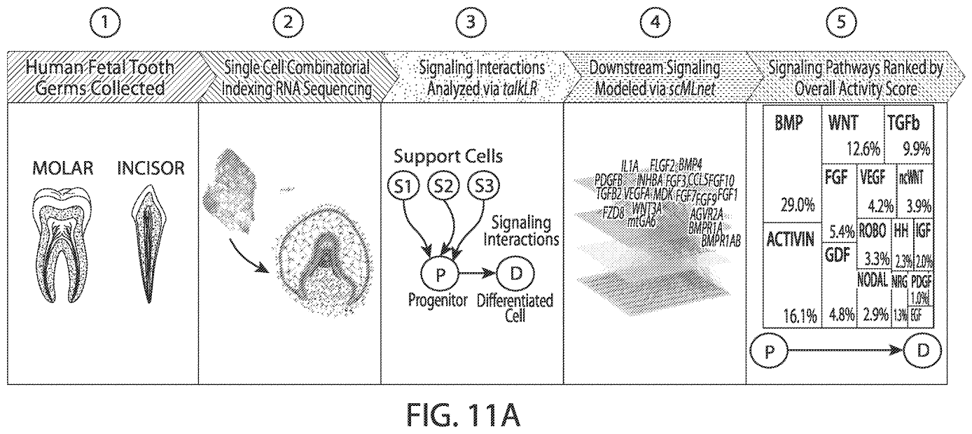

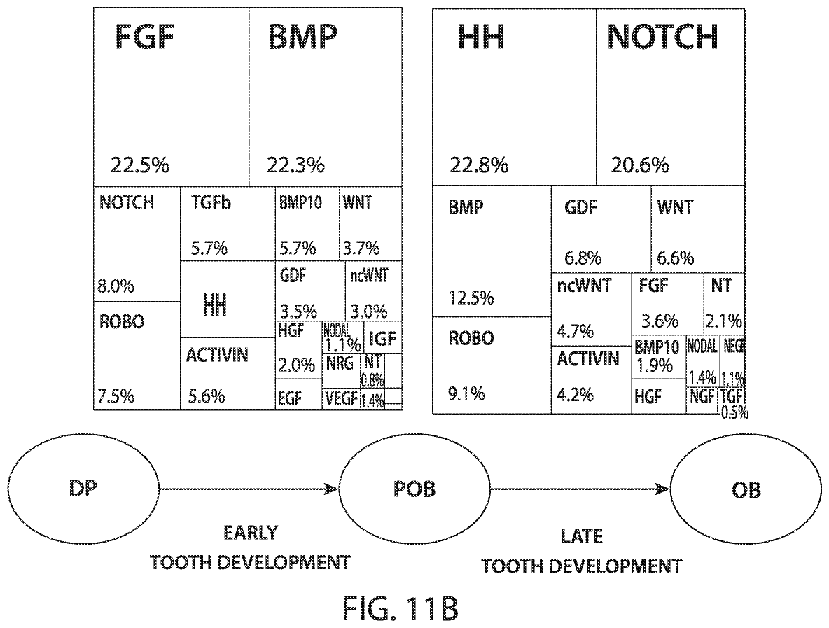

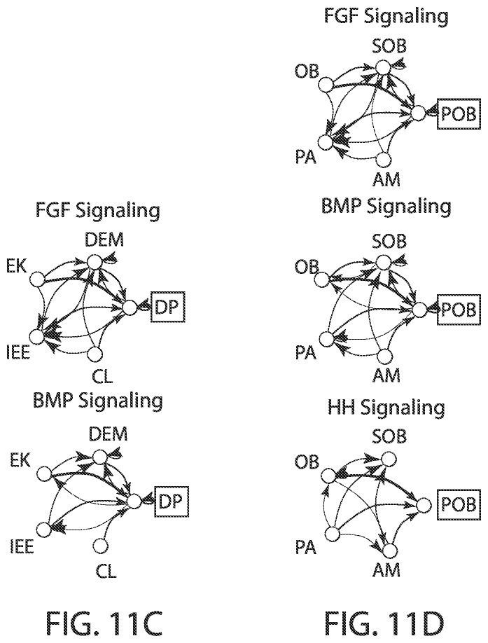

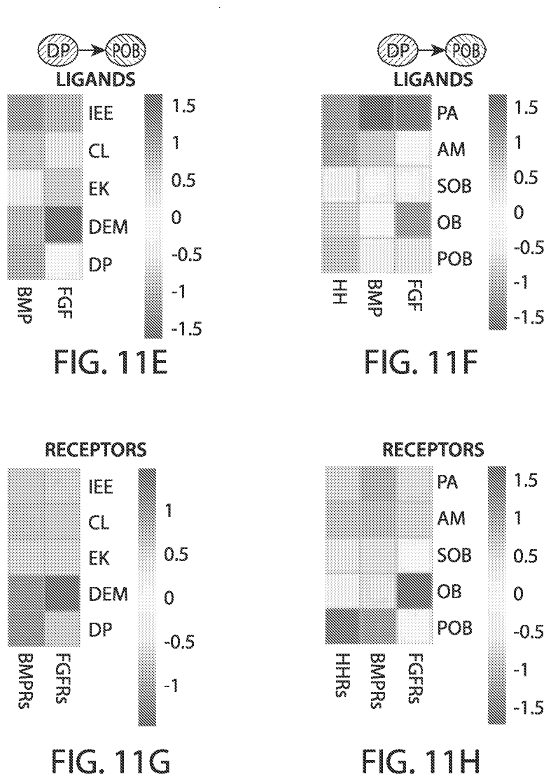

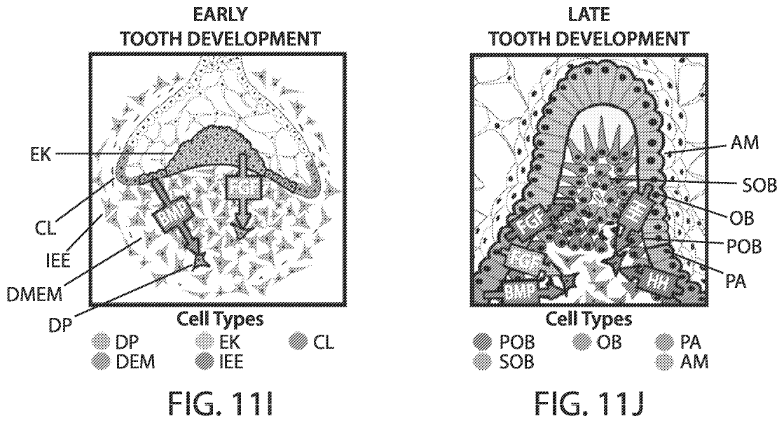

This project applies the DPSC derived to odontoblast protocol previously described by the lab (30), modified for hiPSC and to reflect the full signaling pathway activities as detected by the sci-RNA-seq analysis (FIG. 11A; FIGS. 15B and 15C). In order to fully elucidate the role of FGF signaling in odontoblast development, the de novo designed FGFR1/2c isoform mini binders mb7 were used, which functions as a FGF antagonist, and C6, which acts as a FGF agonist (FIG. 15A) (31. 32). p75+ iNC were cultured in Odontogenic Medium, which consists of DMEM+Glutamax (Gibco 10566016), 100 nM dexamethasone (Sigma-Aldrich D4902), 10% fetal bovine serum, 5 mM b-glycerophosphate (Sigma-Aldrich G9422), and 50 μg/mL L-ascorbic acid (Sigma-Aldrich A4544) for 14 days (OB). Odontogenic medium was supplemented with 50 ng/mL BMP4 (Stemcell Technologies 78211) for 7 days followed by 25 ng/mL BMP4 (Stemcell Technologies 78211) and 400 nM SAG (Stemcell Technologies 73412) for 7 days (iOB); 100 ng/mL C6 (1,2) for 14 days (iOB C6); 100 ng/mL C6 for 7 days followed by 100 ng/mL mb7 (31,32) for 7 days (iOB C6 to mb7); or 100 ng/mL recombinant basic FGF (Gibco 13256-029) for 14 days (iOB bFGF). All cultures were performed on Matrigel coated plates at a 1:30 dilution and incubated at 37° C. and 5% CO₂ concentration. Each differentiation was performed in triplicate with undifferentiated hiPSC as the negative control.

### Protein Isolation.

Media was aspirated from cell culture plates and the cells were gently rinsed with 1×PBS. Cells were lysed from 35 mm plates with 131 μl of lysis buffer containing 20 mM Tris-HCl (Sigma-Aldrich 1185-53-1) (pH 7.5), 150 mM NaCl, 15% glycerol (Sigma-Aldrich G5516), 1% triton (Sigma-Aldrich 9002-93-1), 3% SDS (Sigma-Aldrich 151-21-3), 25 mM β-glycerophosphate (Sigma-Aldrich 50020-100G), 50 mM NaF (Sigma-Aldrich 7681-49-4), 10 mM sodium pyrophosphate (Sigma-Aldrich 13472-36-1), 0.5% orthovanadate (Sigma-Aldrich 13721-39-6), 1% PMSF (Roche Life Sciences 329-98-6), 25 U benzonase nuclease (EMD 70664-10KUN), protease inhibitor cocktail (Pierce™ Protease Inhibitor Mini Tablets, Thermo Fisher Scientific A32963), and phosphatase inhibitor cocktail 2 (Sigma-Aldrich P5726), respectively, in a tube. Cell lysate was collected in a fresh Eppendorf tube. 43.33 μl of 4× Laemmle Sample buffer (Bio-Rad 1610747) containing 10% beta-mercaptoethanol (Sigma-Aldrich M7522-100) was added to the cell lysate and then heated at 95° C. for 10 min. The boiled samples were either used for Western blot analysis or stored at −80° C.

### Western Blot Assay.

Protein samples were thawed via heat block at 95° C. for 10 min. 30 μl of protein sample per well was loaded and separated on a 4-10% SDS-PAGE gel for 30 minutes at 250 Volt. The proteins were then transferred on a nitrocellulose membrane for 12 minutes using the semi-dry turbo transfer Western blot apparatus (Bio-Rad). Post-transfer, the membrane was blocked in 5% bovine serum albumin (BSA) for 1 hour. After 1 hour, the membrane was probed with the primary antibodies Nestin (Santa Cruz SC-23927), DSPP (Santa Cruz 7363-2), RUNX2 (Abcam Ab76956) and GAPDH (Cell Signaling Technology 5174S), overnight on a rocker at 4° C. The following day, membranes were washed with 1×TBST 3×5-minute washes. The respective HRP-conjugated secondary antibodies (Bio-Rad) were added at 1:10,000 dilution and incubated at room temperature for 1 hour. Membranes were then washed with 1×TBST 3×5-minute washes. Membranes were then developed using Chemiluminescence and imaged using Bio-Rad ChemiDoc Imager.

### Immunofluorescence Staining and Confocal Imaging.

Cultured cells were fixed in 4% paraformaldehyde (PFA) then immersed in 1× phosphate-buffered saline (PBS) for 3×5-minute washes. Slides were blocked for 60 minutes at room temperature in a humidified chamber with a blocking buffer consisting of 0.1% Triton X-100 and 5% Bovine Serum Albumin (BSA) (VWR). The primary antibodies DSPP (1:50, Santa Cruz 7363-2), RUNX2 (1:50, Abcam Ab76956), Phalloidin (1:100, Thermo Fisher Scientific A12379) and Vimentin (1:100, Cell Signaling Technology 5741) were incubated overnight at 4° C. in a humidified chamber. After 3×5-minute washes in PBS in a coplin jar, the slides were transferred to a humidified chamber with secondary antibodies. Secondary antibody goat anti-mouse 488 (1:250, Molecular Probes) was applied for 60 minutes at room temperature in the same blocking agent. Slides were then rinsed with PBS 4×10-minute washes in a coplin jar. DAPI (1:50, Molecular Probes) was applied for 10 minutes at room temperature in PBS. Slides were then rinsed with PBS for 5 minutes in a coplin jar. Slides were then mounted with Vectashield (Vector Labs) and stored at 4° C. until used for imaging. Confocal Imaging was done on a Leica TCS-SPE Confocal microscope using a 40× objective and Leica Software. Images were processed with Fiji software distribution of ImageJ v1.52i (33). Negative controls were performed substituting PBS.

Real-Time Quantitative Reverse Transcription-Polymerase Chain Reaction (qPCR).

To analyze gene expression, cells were dissociated and lysed with Trizol (Life Technology) with cell pellets stored at −80° C. RNA purification is performed via TURBO DNA-Free™ Kit (Invitrogen) or Aurum™ Total RNA Mini Kit (Bio-Rad), purity and concentration quantification via Nanodrop ND-1000 (Thermo Fisher Scientific). cDNA synthesis via iScript™ cDNA Synthesis Kit (Bio-Rad) or Applied Biosystems™ High-Capacity cDNA Reverse Transcription Kit (Thermo Fisher Scientific), and qPCR performed using oligonucleotide primers for neural crest and odontoblast markers (Table 3), SYBR Green reporter (Applied Biosystems) and 7300 Real-Time PCR System (Applied Biosystems). All qPCR reactions were performed in triplicate, normalized to R-actin and hiPSC, and assessed using the comparative change in threshold cycle (ΔCₜ) method.

### Statistical Analysis.

ΔΔCₜ values of gene expression of differentiated samples are calculated by normalizing to hiPSC derived odontoblast samples (iOB) and analyzed for significance using Student's t test via GraphPad QuickCalcs (graphpad.com) for comparisons of two samples or with One-way Anova with Bonferroi's multiple comparison tests for comparison of more than two samples in Prism, GraphPad.

### Alizarin Red Stain Mineralization Capacity Assay.

Alizarin red staining (ARS) (Sigma-Aldrich TMS-008) is performed to assess extracellular calcium deposition. Culture medium was aspirated from each well and cells washed with PBS 3×. Cells were fixed in 4% PFA for 15 minutes at room temperature. PFA was removed and cells washed 3× with diH₂O. diH₂O is aspirated off and 1 mL 2% ARS was added per well. Plates were covered in aluminum foil and incubated at room temperature for 45 minutes with gentle shaking. ARS was removed and cells washed 5× with diH₂O. Staining was visualized under phase contrast microscopy (Olympus IX70 microscope, Japan). Stain was then released with 10% acetic acid and neutralized with 0.1 μM ammonium hydroxide and quantification for OD405 performed via Wallac EnVision system.

### Results

The intercellular signaling was investigated on a single cell level between human odontoblast and ameloblast lineages based on recent fetal tooth germ sci-RNA-seq analysis (9). First, the signaling pathways involved in human odontoblast development were analyzed. Second, the signaling pathways were dissected, distinguishing two different human tooth types, incisors and molars, in enamel knot and ameloblast development. Third, the information on the critical signaling pathways involved in human odontoblast differentiation was utilized to develop a hiPSC derived odontoblast differentiation protocol (iOB) using de novo designed FGFR1/2c isoform mini binders (3132).

Single Cell Sequencing Predicts Early FGF and BMP Signaling Followed by Late HH Activation As Crucial To Human Odontoblast Development.

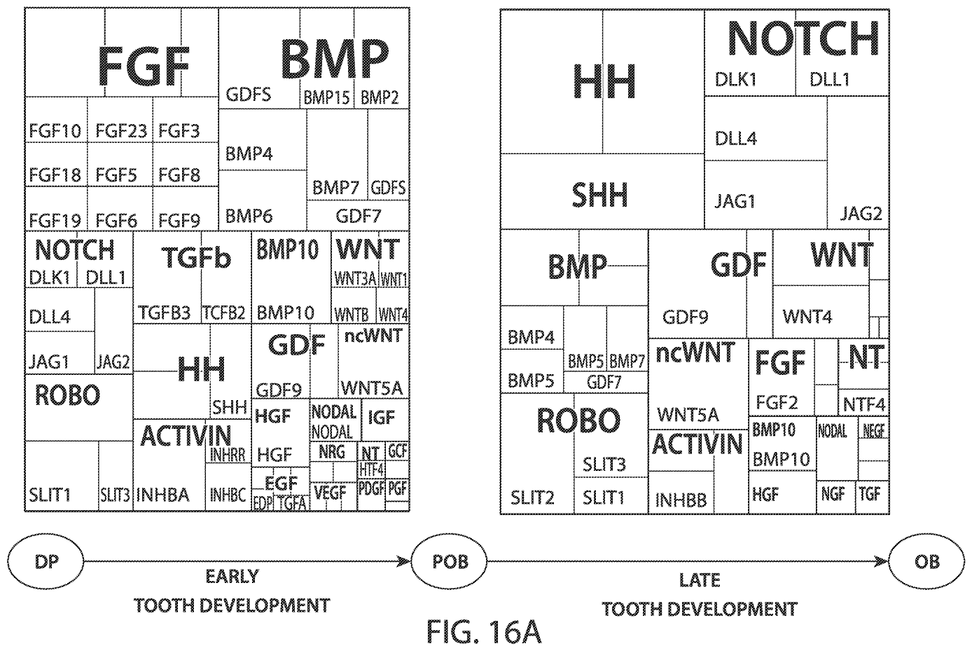

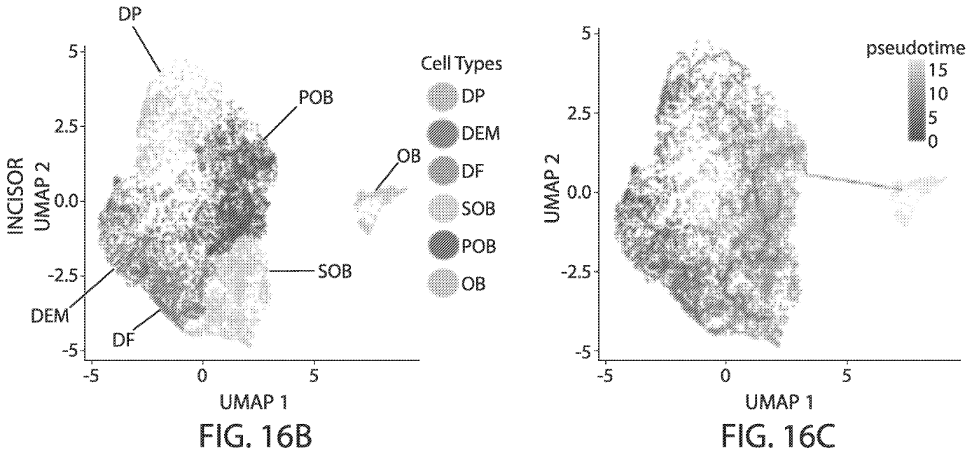

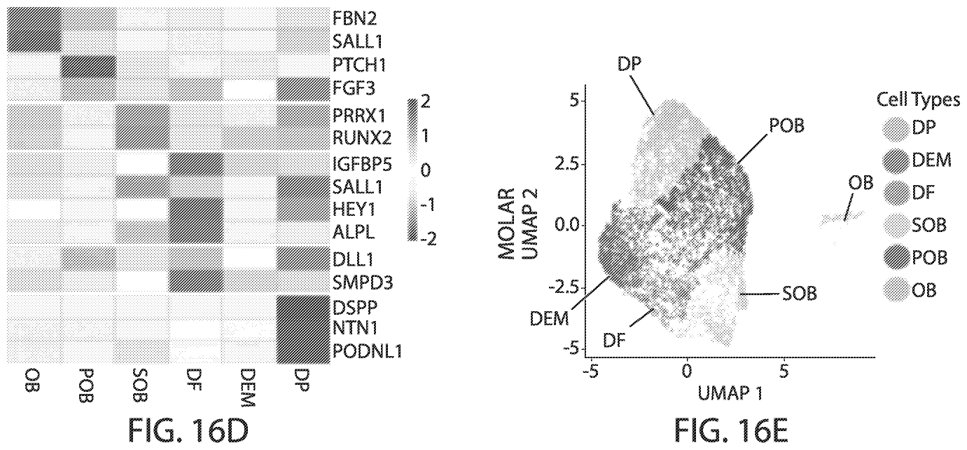

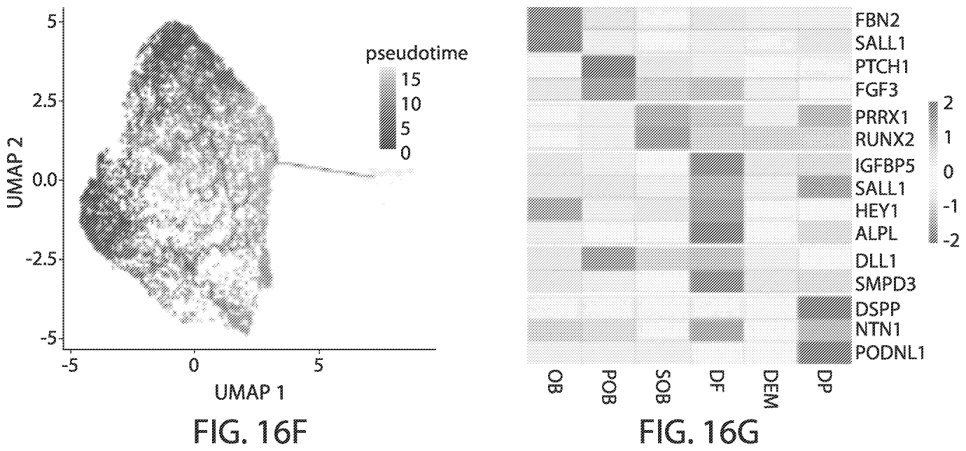

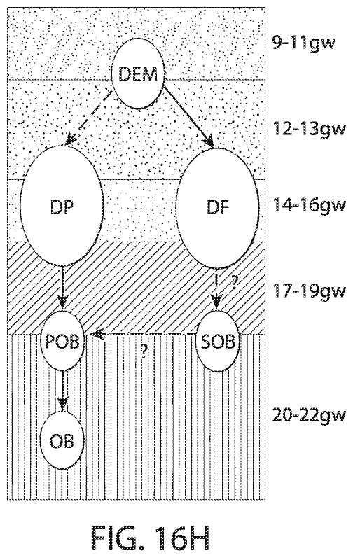

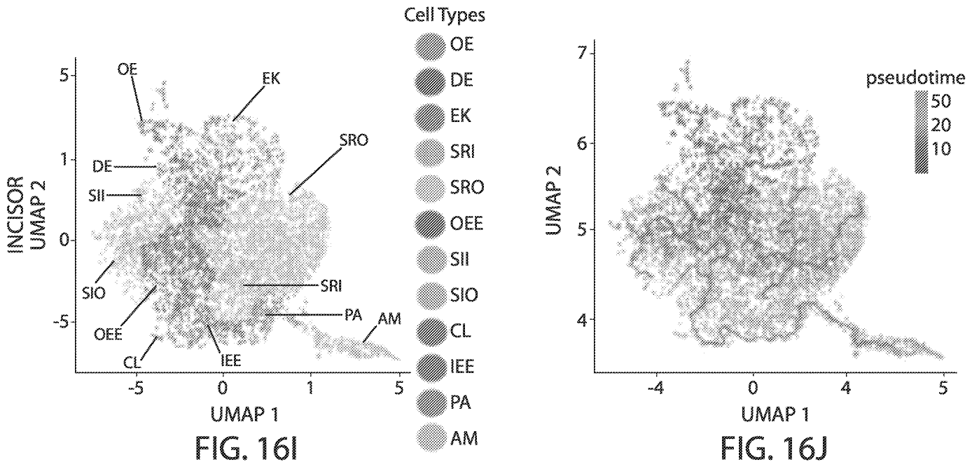

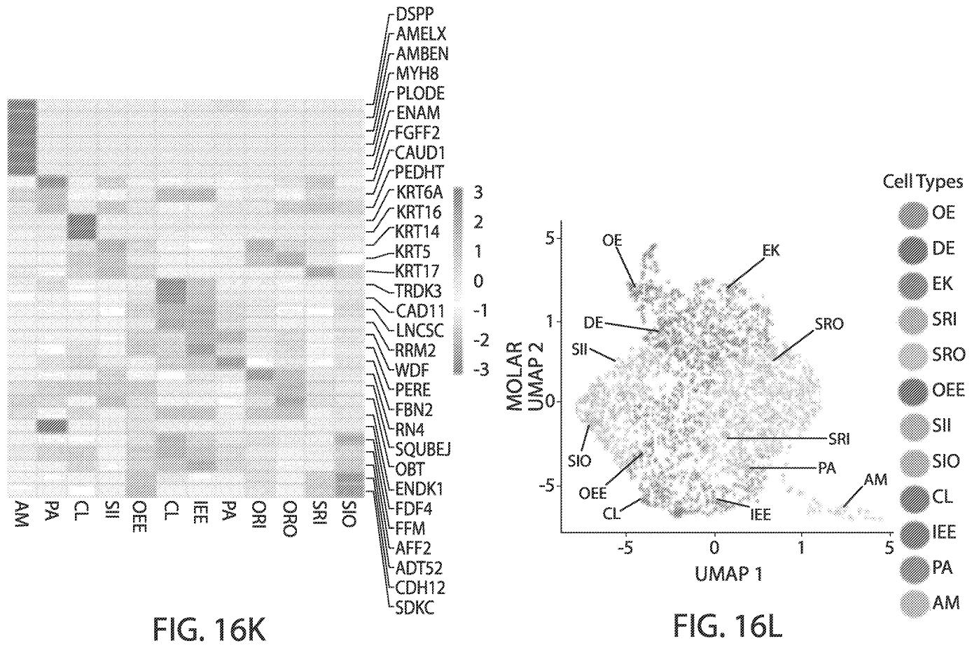

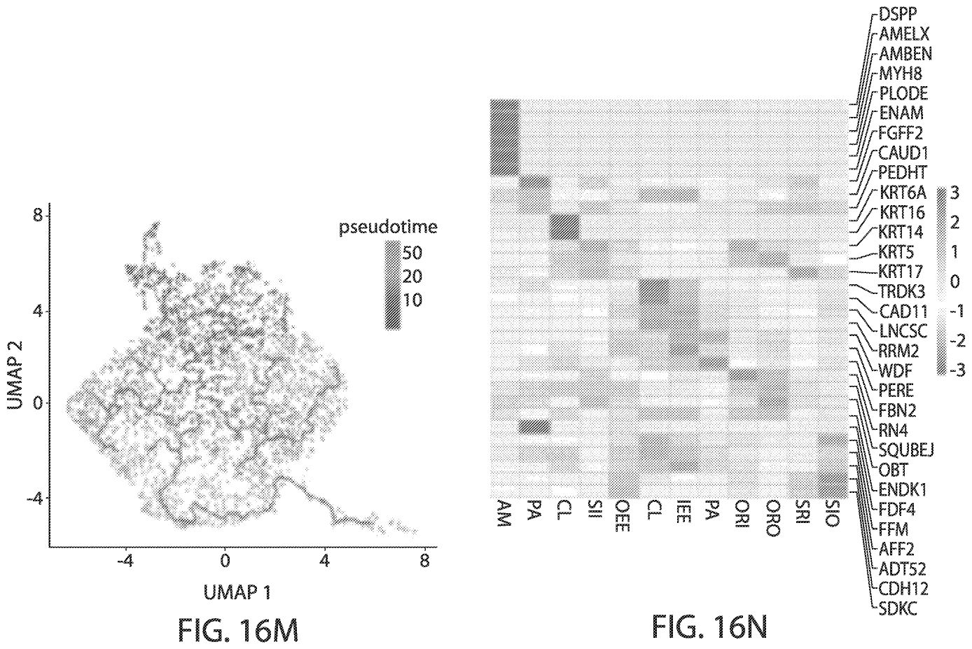

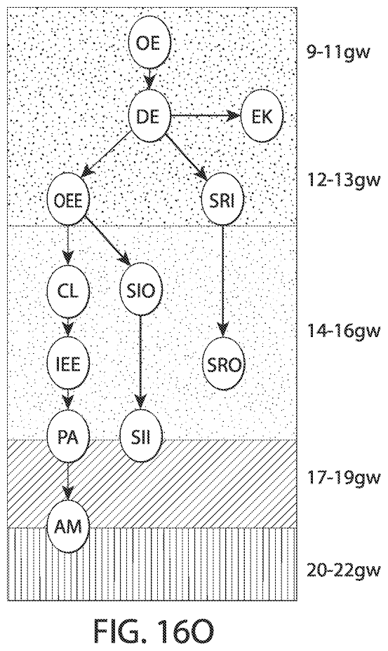

Bioinformatic analysis of the most active signaling pathways during human odontoblast differentiation predicts that fibroblast growth factor (FGF), bone morphogenic protein (BMP), and hedgehog (HH) signaling pathways are critical to directing this developmental trajectory. FGF and BMP are most active during the transition from dental papilla (DP) to preodontoblast (POB) (12-19 gw; FIG. 11A; FIGS. 16A and 16H; Table 4). BMP activity is reduced by roughly half during the transition from POB to odontoblast (OB) with the majority of signaling derived from HH and Notch pathways (17-22 gw; FIG. 11A; FIGS. 16A and 16H; Table 6). During the transition from DP to POB, the dental epithelium derived inner enamel epithelium (IEE) is the major source of BMP signaling ligands (FIGS. 11B and 11D; Table 5), which bind to receptors on the DP (FIG. 11F). FGF signaling ligands are most robustly produced by the enamel knot (EK) (FIGS. 11B and 11D; Table 5) and bind to receptors present on the DP (FIG. 11F). Overall, the dental epithelium derived IEE and EK appear to play critical roles in early human odontoblast development, secreting FGF and BMP ligands and inducing DP differentiation to POB in the tooth germ (FIG. 1H).

During the transition from POB to OB, the main source of BMP, FGF and HH signaling ligands is predicted to be the dental epithelium derived pre-ameloblast (PA) (FIGS. 11C and 11E; Table 7). The receptors for these ligands are highly expressed in the POB (FIG. 1G). HH signaling, the most active pathway in the transition, shows significantly increased ligand expression in both the PA and ameloblast (AM) (FIGS. 11C and 11E; Table 7), which is received by receptors on the POB (FIG. 1G). During late odontoblast development it appears that the dental epithelium derived PA is largely responsible for BMP, FGF and HH ligands secretion, which bind to receptors present on the POB, inducing the transition of human POB to OB (FIG. 1I).

### Human Molar Tooth Development Is Delayed Compared To Incisors.

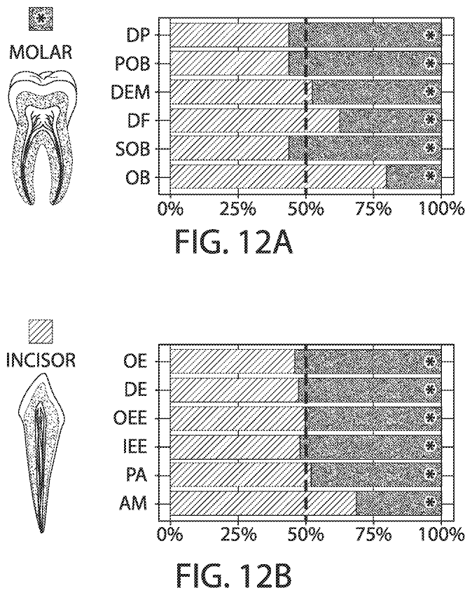

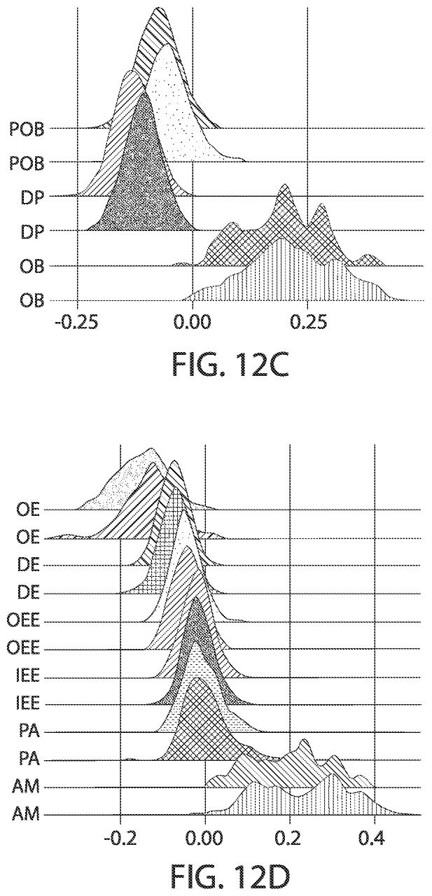

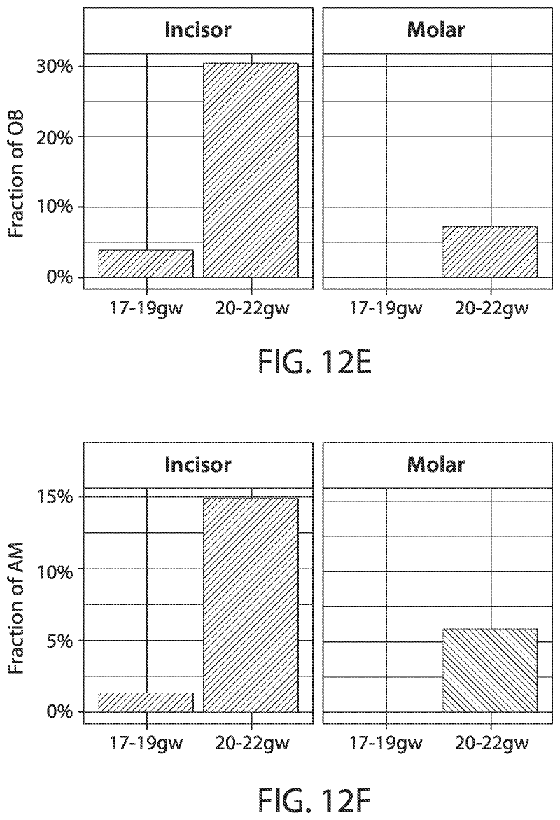

Previous studies have proposed that molars develop more slowly than incisors due to the molar's later eruption date in vivo (34). To investigate this, comparative analysis was performed independently on the developing human incisor and molar tooth germ types. The dataset of single cell RNA sequencing of the oral cavity (9) was subset for the dental ectomesenchyme (FIGS. 16B and 16E) and epithelium lineages (FIGS. 161 and 16L) by incisor and molar tooth germ type. In the dental ectomesenchyme, incisor and molar subsets showed maintained presence of 6 transcriptionally unique cell types (FIGS. 16B, 16E, 16D, and 16G) and 12 transcriptionally unique cell types in the dental epithelium (FIGS. 16I, 16L, 16K, and 16N). Pseudotime trajectories were also consistent between incisor and molar tooth germ types in both dental ectomesenchyme (FIGS. 16C and 16F) and dental epithelium derived tissues (FIGS. 16J and 16M). Simplified differentiation trajectories illustrate in the dental ectomesenchyme, a common dental ectomesenchyme (DEM) progenitor gives rise to both the DP and the dental follicle (DF). In the odontoblast lineage, DP gives rise to POB, followed by OB. In the dental follicle lineage, the DF gives rise to the subodontoblast (SOB), with a suggested transition of SOB through POB-like state before giving rise to OB (FIG. 16H). In the dental epithelium, the ameloblast lineage consists of the dental epithelium (DE), which gives rise to the outer enamel epithelium (OEE) followed by the cervical loop (CL), IEE, PA and finally AM. In this study of ameloblast development, CL and IEE cell types were excluded from analysis as molar and incisor tooth germ tissues had been combined prior to single cell RNA sequencing. The enamel knot lineage shares the same progenitor as the ameloblast lineage, with DE giving rise to the enamel knot (EK) (FIG. 16O).

The proportion of each cell type present in the developing tooth germ at various developmental stages can define the maturation kinetics. Therefore, molar and incisor tooth germ type development was further inspected on the molecular level by analyzing the proportion of each cell type present in the developing human dental epithelium and dental ectomesenchyme derived tooth tissues. In the dental ectomesenchyme derived tissues, progenitor populations including DP, POB and SOB were richer in the molar while mature DF and OB populations were greater in the incisor. DEM populations were roughly equal. (FIG. 12A). Similarly, in the dental epithelium derived tissues, progenitor populations OE, DE and IEE were greater in the molar while mature AM populations were denser in the incisor. OEE and PA populations were roughly equal. (FIG. 12B).

The differentiation state of each cell type was compared by assigning developmental scores. Scores were calculated by selecting marker genes to determine the maturation (e.g. OB and AM) and progenitor state (e.g. DP and IEE) of each cell type, respectively. The difference between these two sets of scores determines the overall developmental score of each cell type. The results indicate no notable developmental delays between cell types regardless of tissue or tooth germ type (FIGS. 12C and 12D). The gestational week (gw) at which OB and AM populations first become present is delayed in molars. While OB and AM was observed at 17-19 gw in incisors, they do not appear in molars before 20-22 gw (FIGS. 12E and 12F). Together these findings show on a transcriptional level that odontoblasts and ameloblasts develop more rapidly in the incisors compared to the molars, with the molars developing overall at a delayed rate compared to the incisors.

Human Enamel Knot Development Is Guided by FGF and WNT In The Incisor and BMP and ROBO In The Molar, With The Dental Ectomesenchyme As The Mutual Primary Source of Signaling Ligands.

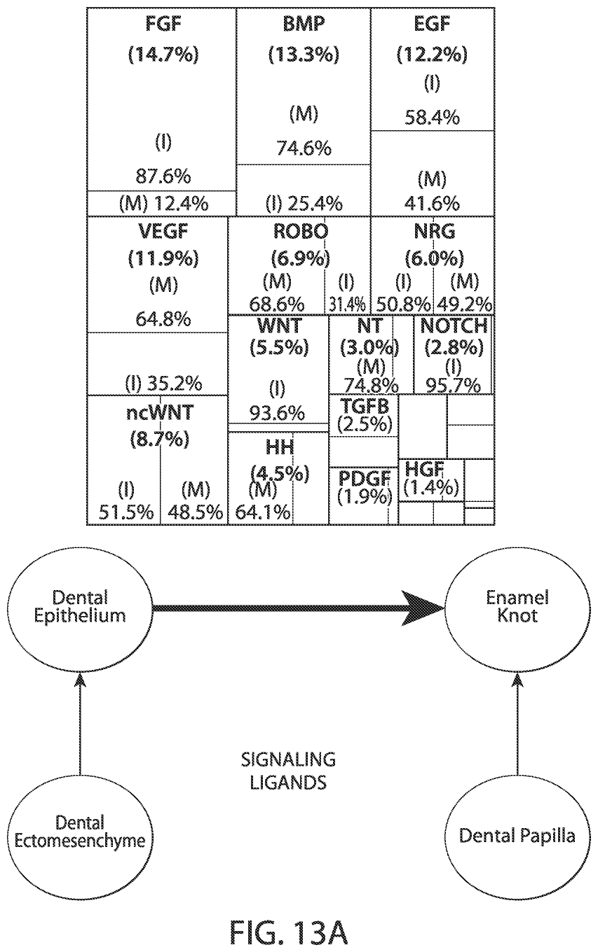

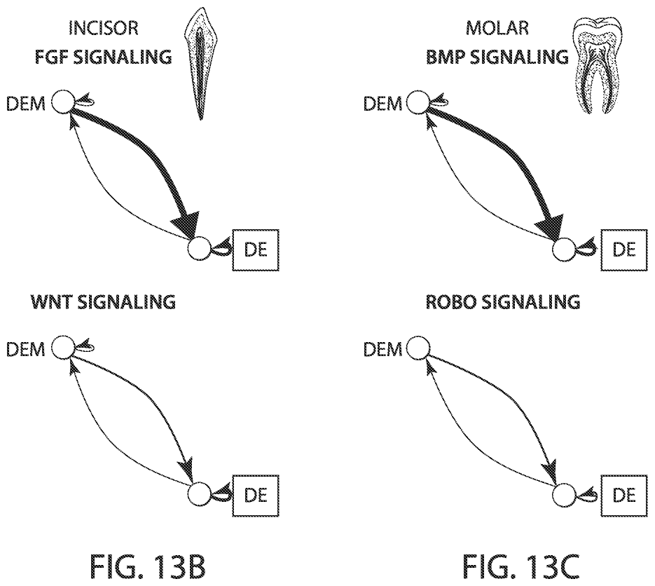

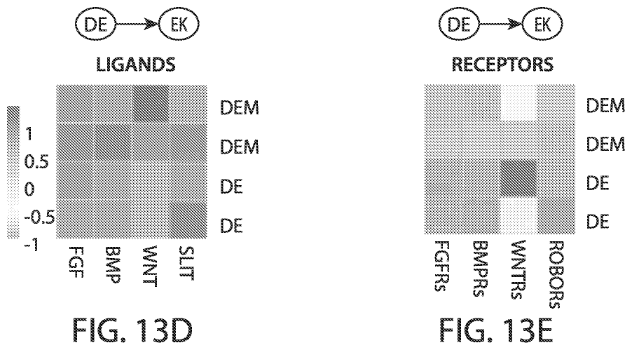

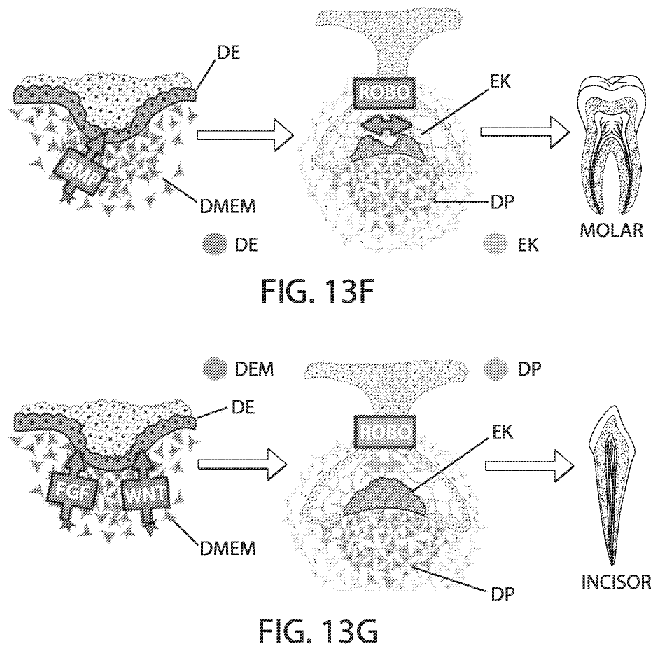

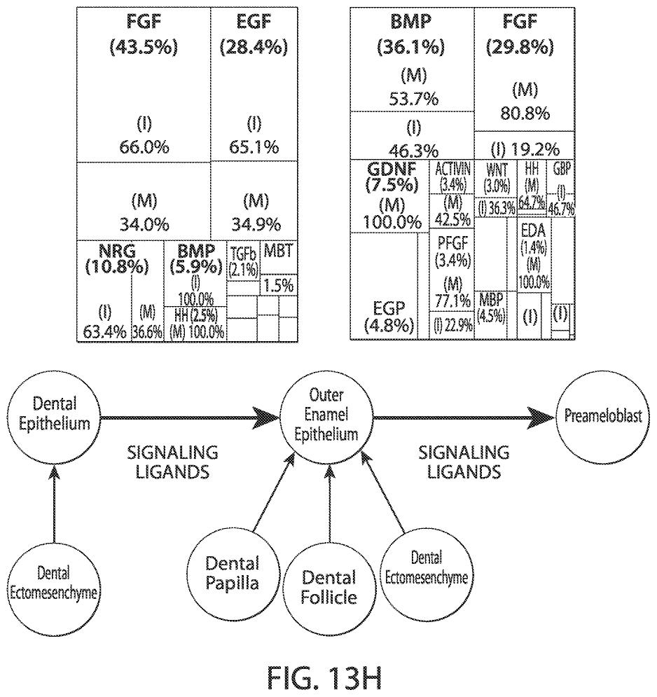

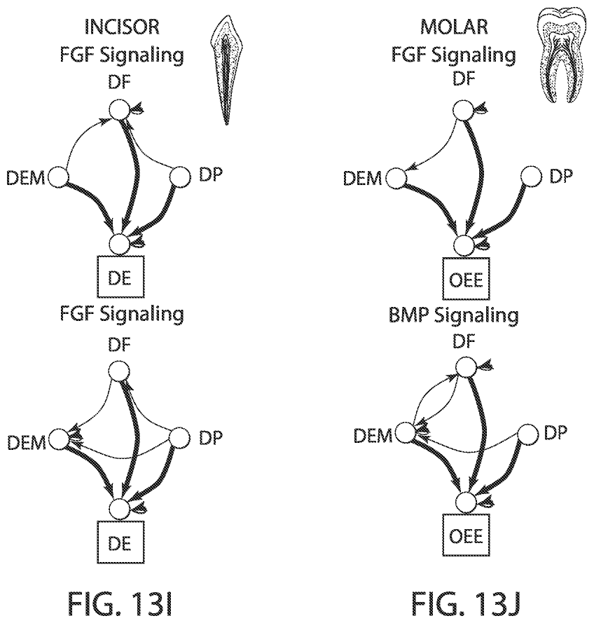

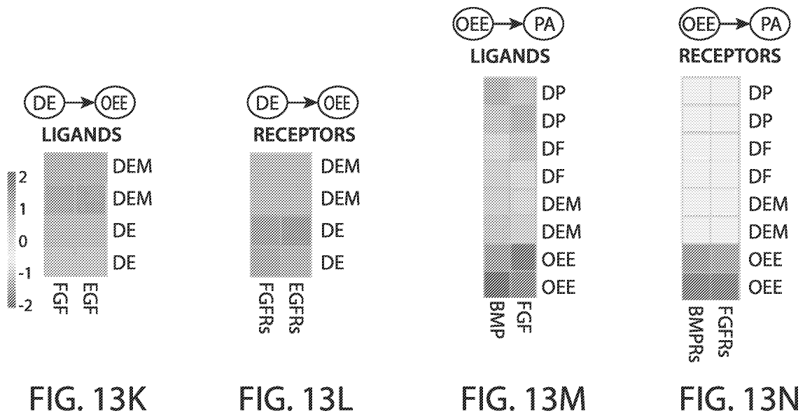

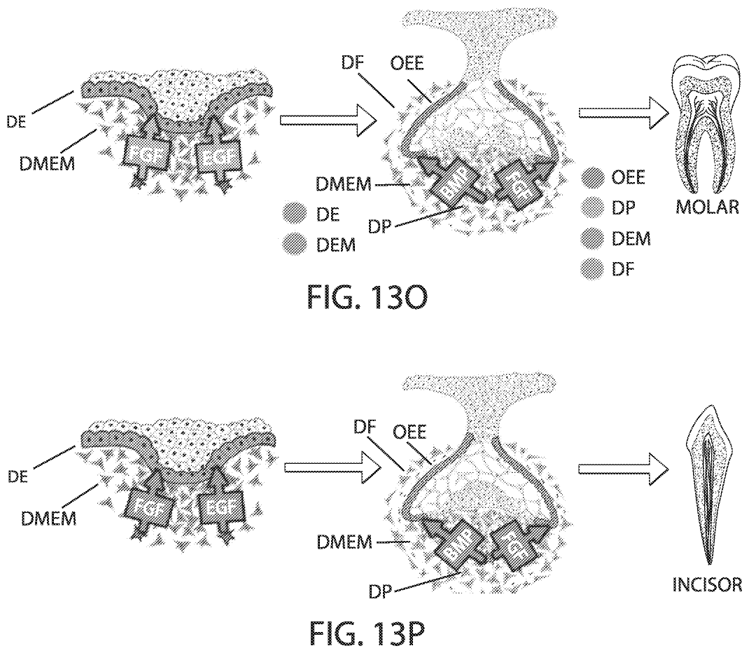

Comparative sci-RNA-seq computational analysis of the signaling interactions that guide human enamel knot (EK) development from the dental epithelium (DE) (FIG. 13A; FIG. 16O) predicts several important differences in between incisor and molar tooth germ types. Incisor enamel knot development requires approximately seven times greater FGF signaling and 14 times more WNT signaling than molars. In contrast, molar enamel knot development requires roughly three times greater BMP signaling and two times more ROBO signaling than incisors (FIG. 13A; Table 8). During the transition from DE to EK, in both incisor and molar tooth germ types, the enamel knot is vastly activated by ligands produced by the dental ectomesenchyme (DEM) (FIGS. 13B-13D; Table 9). A notable exception is the ROBO ligand SLIT, which is highly expressed in the DE (FIGS. 3C-3D; Table 9). Elevated FGF and WNT ligand production by the DEM (FIGS. 13B and 13D; Table 9) and FGF receptors (FGFRs) and WNTRs in the DE are observed in the incisor (FIGS. 13E and 13G). Comparatively, elevated BMP ligand production by the DEM is observed in the molar (FIGS. 13C-13D; Table 9), with increased BMPRs on the DE (FIGS. 13E-13F). Increased SLIT ligand production is seen in the molar DE (FIGS. 13C-13D; Table 9), with receptor ROBO expression isolated to the DE (FIGS. 13E-13F), indicating autocrine ROBO signaling within the developing molar enamel knot. These bioinformatics-based predictions suggest BMP and ROBO signaling pathway crosstalk with ROBO ligand SLIT acting as a BMP target. As ROBO signaling is well recognized for guiding axon migration through repulsive action (35), higher levels of autocrine ROBO signaling within the molar enamel knot plays a similar role, providing repellent patterning for the formation of secondary enamel knots distanced from the site of the primary enamel knot.

Incisor and Molar Ameloblast Development Is Guided By FGF, EGF and BMP Activation With Tooth Type Specific Intensity of Signaling Interactions. Dental Ectomesenchyme Derived Cells Act As The Shared Primary Source of Signaling Ligands.

Comparative analysis of the signaling interactions that guide human ameloblast development from dental epithelium (DE) to outer enamel epithelium (OEE) followed by preameloblast (PA) (FIGS. 13H; 16O) predicts several important differences between incisor and molar tooth types.

Incisors require approximately two times greater FGF and EGF signaling during the transition from DE to OEE compared to molars (Table 10). Molars require elevated BMP and four times greater FGF signaling during the maturation of OEE to PA (FIG. 13H; Table 12). During the transition from DE to OEE, the incisor DE is vastly activated by EGF ligands produced by the dental ectomesenchyme derived dental follicle (DF) and FGF ligands produced by the dental papilla (DP) (FIGS. 13I, 13K, and 13P; Table 11), which bind to receptors on the DE (FIGS. 13L and 13P). During the transition from OEE to PA, the molar OEE is robustly activated by BMP and FGF ligands produced by the DP (FIGS. 13J, 13MM, and 13O; Table 13), with receptors located on the OEE (FIGS. 13N-13O). These bioinformatics-based predictions suggest that FGF and EGF signaling are critical for early ameloblast development in the incisor (FIG. 13P), while increased FGF and BMP activation are required for ameloblast maturation in the molar (FIG. 13O). Dental ectomesenchyme cells are largely responsible for secretion of the signaling ligands which activate these pathways (FIGS. 13O-13P).

Early FGF and BMP Activation with Late FGF Agonism Using the De Novo Designed FGFR1/2c Isoform Mini Binder C6 and HH Activation Leads To More Mature hiPSC Derived Odontoblast Differentiation In Vitro (iOB).

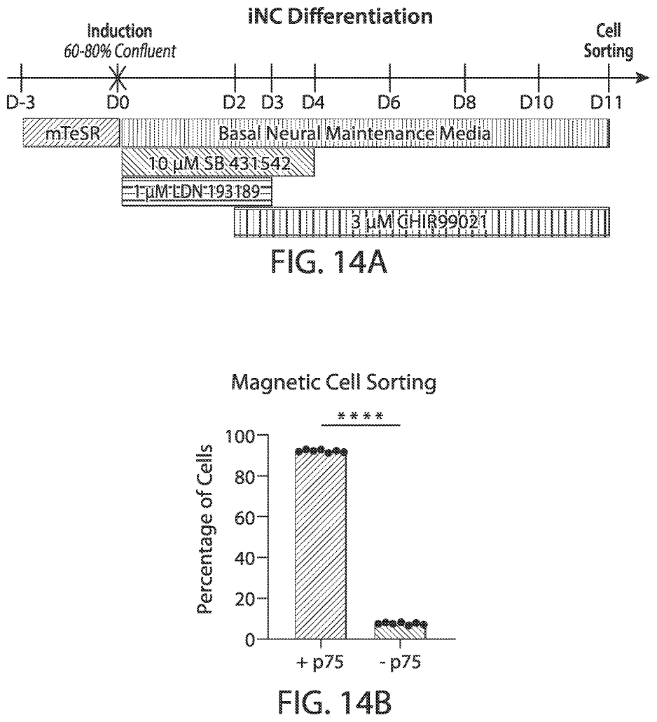

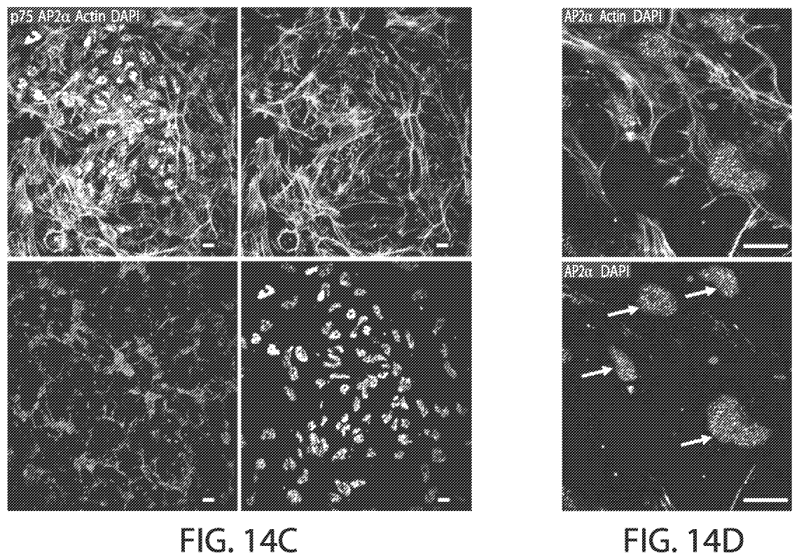

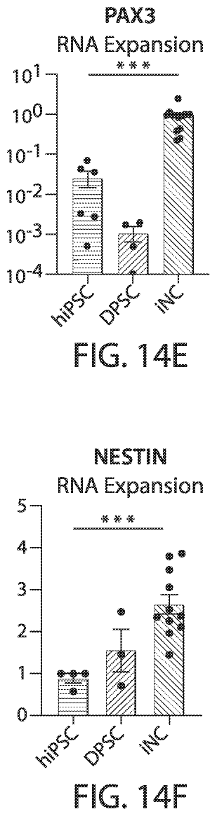

Using the pathways predicted by single cell sequencing analysis of the developing human tooth germ are truly critical for odontoblast development (FIG. 11A; 16A), a human induced pluripotent stem cell (hiPSC) derived odontoblast differentiation protocol was designed by activating FGF, BMP, and HH signaling at appropriate developmental stages. To fully capture the developmental trajectory of the human odontoblast hiPSC were first differentiated hiPSC to a neural crest fate (FIG. 14A) as described previously (27-29). Successful differentiation was confirmed by magnetic cell sorting for neural crest marker p75, with 90% of cells sorted positively expressing p75 (FIG. 14B). Differentiation was further validated by immunofluorescence analysis, which shows induced neural crest cells (iNC) express neural crest markers p75 and transcription factor AP-2α (FIGS. 14C-14D). Expression of neural crest markers NESTIN (36) and PAX3 (37) were next assessed at the transcriptional level via qPCR. A significant 4- and almost 100-fold increase in expression of NESTIN and PAX3 was observed, respectively, in iNC compared to undifferentiated hiPSC (FIGS. 14E-14F).

Next, iNC were biased to an odontoblast fate by culture in odontogenic media as observed in conventional odontoblast differentiation protocols (OB) (30). To activate the BMP and HH signaling pathways identified by computational analysis of sci-RNA-seq data (FIGS. 11A; 16A), odontogenic media was supplemented with BMP ligand BMP4 and HH pathway agonist SAG (iOB). In order to elucidate the role of FGF signaling in odontoblast differentiation, as predicted by sci-RNA-seq analysis, odontogenic medium was additionally supplemented with the de novo designed FGFR1/2c mini binder agonist C6 (iOB C6) (31,32); C6 followed by the de novo designed FGFR1/2c mini binder antagonist mb7 (iOB C6 to mb7) (3132) (FIG. 15A); or basic FGF (iOB bFGF) (FIGS. 15B-15C).

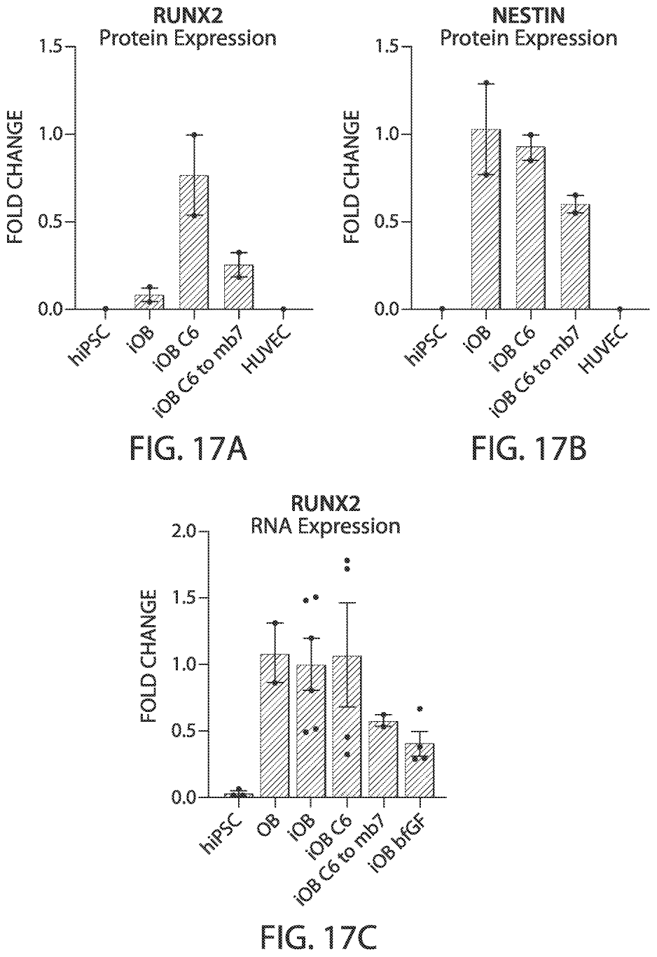

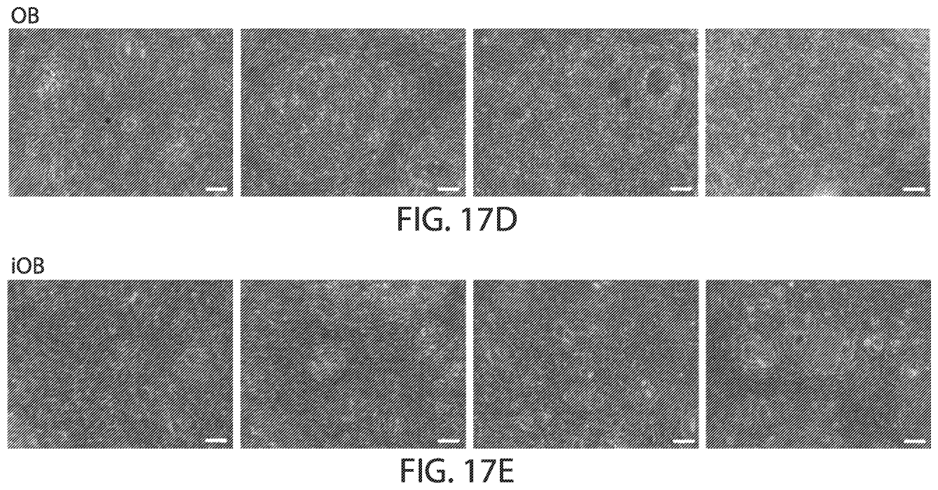

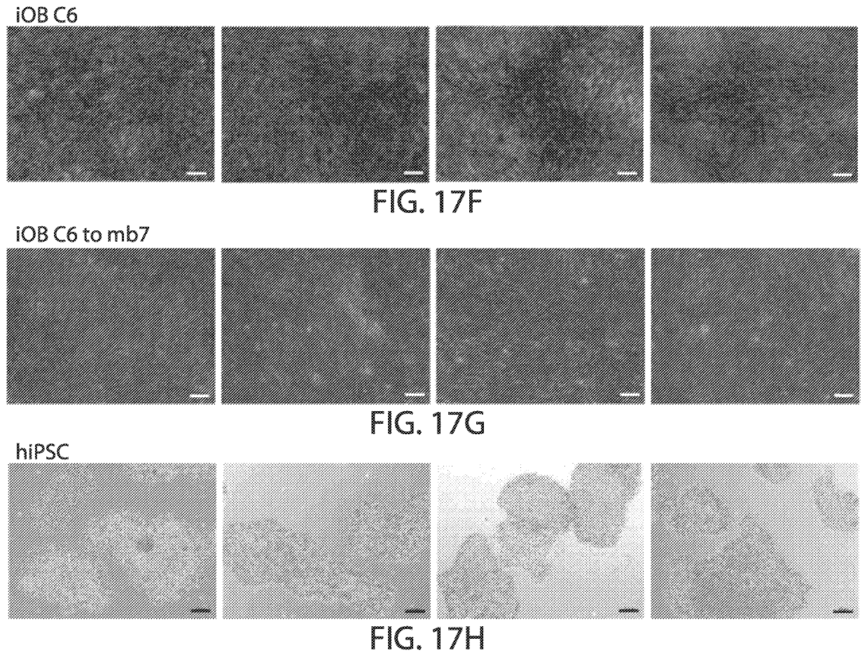

hiPSC derived odontoblast cells (iOB) have increased expression of mature odontoblast markers DSPP (10) and RUNX2 (38) at the protein level as assessed by Western Blot (FIG. 15D). Further, iOB treated with C6 (iOB C6) show a significant two-fold increase in DSPP expression compared to both iOB and iOB C6 to mb7 cells (FIGS. 15D-15E). No significant change in RUNX2 or NESTIN was observed at the protein level (FIGS. 15D; 17A-17B). Successful differentiation of iNC to an odontoblast fate was further validated by immunofluorescence analysis, which shows iOB C6 cells most strongly express odontoblast markers DSPP and RUNX2 compared to OB, iOB, and iOB C6 to mb7 (FIG. 15F). Expression of odontoblast markers RUNX2, DSPP, and DMP1 were next assessed at the transcriptional level via qPCR, which indicates significantly greater expression of both mature odontoblast markers DSPP and DMP1 in iOB C6. Compared to iOB and iOB bFGF, iOB C6 cells show 3- and 5-fold increases in expression of DSPP and DMP1, respectively (FIG. 15G-15H). No significant changes in RUNX2 expression were observed (FIG. 17C). The de novo designed mini binders interact exclusively with the FGFR1/2c isoform, allowing differential functional analysis of the FGFR1/2c- and b-isoforms (31,32). There was a significant increase in FGFR1c expression in iOB and iOB C6 treated cells (FIG. 15I). Lastly, biomineralization capacity was assessed via Alizarin Red Staining. iOB C6 shows significantly enhanced deposition of mineralized matrix compared to iOB, as evidenced by increased mineralized nodule formation, while iOB C6 to mb7 show a significant decrease in biomineralization capacity compared to iOB C6 (FIG. 15J-15L; FIGS. 17D-17H).

### DISCUSSION

### Roundabout Signaling Predicted To Shape Molar Enamel Knot Formation.

Tooth development requires continual, reciprocal signaling between the dental epithelium and dental ectomesenchyme derived tissues (5). Isolated tooth epithelium or dental ectomesenchyme do not result in tooth formation (6). Odontogenic potential shifts from dental epithelium to dental ectomesenchyme at the cap stage of tooth development (11). However, whether determination of tooth type (e.g. if an incisor or molar will form) lies with the dental ectomesenchyme or dental epithelium remains unknown. Previous studies show the enamel knot is a critical receiving cell for tooth type determination, triggering proliferation of neighboring dental ectomesenchyme cells and epithelium derived cervical loop cells (12). The signaling determinants of enamel knot formation were revealed in the incisor compared to the molar. This analysis indicates WNT and FGF are the most active signaling pathways in incisor enamel knot development, with ligand secretion largely from the dental ectomesenchyme. FGF4 and SLIT1 are currently the best biomarkers for the developing murine enamel knot, as they are the sole genes observed to be expressed in both primary and secondary enamel knots (42,43). It was previously shown localized FGF4 expression in the human incisor enamel knot (9), indicating a shared expression pattern between murine and human enamel knot development. While FGF4 is known to stimulate cusp growth by inducing proliferation of dental epithelium and ectomesenchyme derived cells, the role of SLIT/ROBO signaling in enamel knot development is not fully understood.

Molar enamel knot formation is predicted to be guided by BMP ligand production by the neighboring dental ectomesenchyme, followed by ROBO activation in the dental epithelium. BMP-SLIT crosstalk has been observed in myoblasts and fibroblasts (44). The ROBO ligand SLIT is a BMP target in the dental epithelium, activation of which results in increased ROBO/SLIT activity in molar enamel knot development. SLIT proteins have an evolutionarily conserved role in axon guidance as repulsive ligands for ROBO receptors and are best known for mediating axon migration (35). Increased expression of SLIT ligand and ROBO receptor in molar enamel knot development acts in a similar chemorepulsive fashion, inducing migration of the dental epithelium cells that will give rise to secondary enamel knots, resulting in multiple cusp formation observed in molars. While the cell fate trajectories of primary and secondary enamel knots are not fully understood (45,46), this study illuminates that ROBO/SLIT signaling may play a critical role in molar enamel knot patterning.

### Human Ameloblast Development Relies on Dental Ectomesenchyme Produced Signaling Ligands.

Ameloblast development has been shown to be reliant upon signaling ligands produced by the dental ectomesenchyme derived cells (9). However, the specific cells of the dental pulp and how their signaling interactions with the neighboring dental epithelium derived cells impact ameloblast development remained unknown. Both incisor and molar ameloblasts require FGF, EGF, and BMP signaling during development. FGF and EGF signaling appear critical for early ameloblast development in the incisor, while FGF and BMP are suggested for ameloblast maturation in the molar. The dental ectomesenchyme cells are largely responsible for secretion of the signaling ligands that activate these pathways in both tooth germ types. Crosstalk between BMP and FGF signaling pathways have been shown to influence the site of murine tooth formation, regulating areas of cell proliferation and apoptosis (47). This supports a role for BMP and FGF crosstalk in human ameloblast development, with signaling ligands originating from the dental ectomesenchyme derived cells.

The Need for hiPSC Derived Odontoblasts is Paramount for Regenerative Therapies and Disease Modeling.

Odontoblasts are responsible for the formation of the tooth's dentin, which composes most of the tooth's mineralized tissue. Dentin provides the tooth's toughness, or resistance to crack propagation, and tensile strength, or distribution of biomechanical forces to the surrounding periodontium. While odontoblasts persist throughout life and can respond to injury by secreting tertiary dentin, their number and ability to produce dentin significantly decreases with age, posing a challenge to regenerative dentistry. If the primary odontoblasts are lost, dental pulp stem cells (DPSC) are induced to differentiate into odontoblast-like cells, forming reparative dentin (39). DPSC have previously been shown to successfully differentiate towards osteogenic and odontogenic fates (39-41) and have been characterized by the lab in detail (30). However, DPSC expansion and regeneration capacity is limited (48), showing a dramatic decrease in regenerative capacity with increased age (49).

As the need for odontoblast regeneration is critical, previous studies have explored stem cell derived odontoblast differentiation protocols. In animal models, odontoblast-like cells have been produced from murine iPSC (miPSC) through co-culture with dental epithelium, with the goal of mimicking early tooth development in which the odontoblasts are in proximity with the ameloblasts (50,51); and through gene transfection of miPSC to increase BMP4 and PAX9 expression (52). These methods are not ideal for therapeutic application, as access to developing human oral epithelium is limited and human gene therapy requires further study of off-target effects before being clinically practical. Interestingly, a recent study found supplementation of miPSC derived neural crest-like cells with BMP4, FGF8, and WNT3a increases expression of odontoblast marker genes and odontoblast-like morphology (53), supporting a vital role for these signaling pathways in odontoblast maturity. In humans, BMP4 supplementation has been found to produce more mature iPSC-derived odontoblast-like cells (54). However, this method did not transition through a neural crest state prior to odontoblast differentiation, leaving a gap missing a crucial stage in odontoblast formation and preventing full analysis of odontoblast development needed for regenerative therapies and disease modeling.

### A First-Of-Its-Kind Insight Into The Fate Drivers of Human Odontoblasts.

The studies have revealed, for the first time at the single cell level, the signaling pathways that govern each transition between odontoblast cell lineage identities. Previous studies of hypodontia and tooth agenesis have shown that disruption of fibroblast growth factor (FGF), bone morphogenic protein (BMP), and hedgehog (HH) signals result in defective tooth development. However, the detail with which the study has revealed the role of these pathways at various points in odontoblast development may more mechanistically explain how defects in these pathways lead to tooth loss or tooth agenesis.

The computational analysis identified FGF, bone BMP, and HH signaling to play critical roles in human odontoblast development, with the majority of signaling ligands secreted by neighboring dental epithelium tissues. Odontoblasts are believed to develop through reciprocal, repeated signaling interactions with the dental epithelium derived ameloblasts. The signaling pathway analysis indicates that the majority of signaling ligands critical for odontoblast development are produced by the dental epithelium derived inner enamel epithelium and pre-ameloblast at early and late tooth development, respectively. Interestingly, as the POB transitions to OB, the bulk of BMP signaling ligands received are secreted by the SOB, indicating a supportive role for this novel cell type in human OB development. While previous studies have focused on the role of a single signaling pathway, many others have highlighted the importance of crosstalk between pathways in tooth development and maintenance (55-57). The predictive pathway analysis highlights not only the primary pathway responsible for each stage, but ranks the other pathways involved, meaning that the study will facilitate the investigation into both previously identified and yet undescribed crosstalk in driving forward development. This analysis will facilitate more detailed and informed studies on degenerative dental diseases and can lead to the development of more effective ways to mitigate or reverse tooth loss. This knowledge can be used to develop therapeutic agents to induce dentinogenesis clinically and was applied here to develop an efficient hiPSC derived odontoblast differentiation protocol (iOB).

Single Cell RNA Sequencing Guided Targeting of FGFR1 C-Isoform Using De Novo Mini Binders Produces More Mature hiPSC Derived Odontoblasts In Vitro (iOB).

Analyzing the signaling interactions that guide human odontoblast development allowed to predict the signaling molecules needed to recapitulate odontoblast differentiation in vitro. Importantly, single cell analysis of the odontoblast lineage indicated that BMP, HH and FGF signaling are critical to human odontoblast development. It was found that iNC cultured in odontogenic medium will differentiate towards an odontoblast fate (OB). Activation of sci-RNA-seq detected signaling pathways BMP, HH, and FGF via supplementation with BMP4, SAG, and without (iOB) or with bFGF (iOB bFGF) produces more mature odontoblast cells illustrating increased expression of odontoblast markers DSPP and DMP1 and increased mineralization capacity. Agonism of FGFR1/2c isoform using the de novo designed mini binder C6 produced the most advanced odontoblasts with significantly increased expression of mature odontoblast markers DSPP and DMP1 at both the RNA and protein levels, with significantly enhanced mineralization capacity (iOB C6). Initial FGF agonism followed by antagonism (iOB C6 to mb7) produced cells with similar DSPP and DMP1 expression as iOB C6, but with significantly impaired deposition of mineralized matrix. These findings indicate that while sci-RNA-seq identified BMP and HH signaling play critical roles in early human odontoblast development, it is the agonism of FGF signaling using the de novo designed FGFR1/2c mini binder C6 that produces odontoblast with significantly greater maturation and biomineralization capacity, loss of which results in inhibited mineral deposition activity.

A limitation of single cell RNA sequencing is the insensitivity to splice variants of a given signaling pathway, grouping all isoforms of signaling ligands and receptors under the large umbrella of the overall signaling pathway (9). FGFR1 is known to exist as two alternatively spliced variants, the b- and c-isoforms(58), which are thought to play unique roles in development. To further elucidate the role of FGFR1 splice variants in human odontoblast development, the de novo designed mini binder was utilized (referred to as mb7) which binds the FGFR1/2c isoform with high specificity (31,32). Previous studies have shown that clustering FGFR1/2c by directly fusing mb7 to a cyclic, homo-oligomeric, hexameric scaffold (referred to as C6) generates FGF signaling pathway agonism targeting the FGFR1/2c isoform exclusively (31-32). iNC was exposed to odontogenic medium containing equivalent concentrations of bFGF, which indiscriminately activates both FGFR1/2c and FGFR1/2b, and C6. It was found that while iOB bFGF cells show increased DSPP and DMP1 expression compared to cells treated with a conventional odontoblast differentiation method (OB), iOB C6 cells have significantly higher expression of these mature odontoblast markers compared to iOB bFGF, in addition to more robust mineralization capacity indicated by greater mineralized nodule formation. Intriguingly, iOB C6 cells show high expression of FGFR1c compared to FGFRlb, indicating that FGFR1c is the prevalent isoform in odontoblasts and supporting C6's previously reported role as a FGF signaling pathway agonist (31,32). Thus, the findings suggest that FGFR1c is upregulated in functional odontoblasts and specifically plays a crucial role in driving odontoblast maturity (59,60,61) rather than odontoblast progenitor proliferation (62).

### iOB Impact On Disease Modeling.

The hiPSC derived odontoblast differentiation protocol guided by single cell RNA sequencing utilizing the de novo designed FGFR1/2c mini binder C6 now reveals a novel, highly simplified method to identify the precise signaling pathways required during the stages of human odontoblast development. The method described in this study, using the de novo mini binders to unravel the FGF signaling required for odontoblast maturation in humans, will be generally applicable and specific to any signaling pathway analyzed in the differentiation of normal and disease organoids. This finding implies great potential for de novo designed mini binders as therapeutic agents to induce odontoblast differentiation in clinical cases of pulp exposure or deep caries, as well as generation of mature iOB to be used for tooth organoid generation. Beyond bioengineering lost dentin tooth structure, a method of producing functional odontoblasts from hiPSC serves as a model essential to studying genetic diseases affecting dentin formation. This includes Tricho-Dento-Osseous (TDO) syndrome, a rare but highly penetrant autosomal dominant disorder associated with mutations in the homeodomain transcription factor DLX3 (63), producing debilitating dental defects leading to increased incidence of dental caries, tooth fracture, pulpal necrosis, and tooth loss. In order to develop TDO therapies, it is critical to deepen the understanding of DLX3's role in human odontoblast development.

### CONCLUSION

Insights at the single cell level into the signalling interactions guide human odontoblast and ameloblast development, as well as those that determine incisor and molar tooth type identity. A novel role has been shown for ROBO chemorepulsive signaling in molar enamel knot formation and share knowledge of the signaling patterns that guide enamel knot development in the incisor and molar, allowing specification of cusp formation and crown morphology in forthcoming organoid studies. Analysis of the signaling patterns predicted from sci-RNA-seq of the developing human tooth germ generates a hiPSC derived odontoblast differentiation method utilizing the de novo designed FGFR1/2c mini binder (iOB C6). This study marks the first application of artificial intelligence optimized proteins in the field of regenerative dentistry and provides a profound tool to be used for therapeutic and disease modeling goals. The findings support a functional role for FGFR1c isoform in human odontoblast maturation. Co-culture studies of iOB C6 with the previously described hiPSC derived ameloblasts (iAM) (9) will allow further dissection of the signaling patterns exchanged between dental epithelium and dental ectomesenchyme derived tissue types during tooth development, likely driving advanced maturation of both odontoblast and ameloblast cell types.

### Tables

TABLE 3
qPCR Primer Sequences.

| | | Forward | Reverse |
|---|---|---|---|
| | | Sequence | Sequence |
| Cell Type | Primer | (5¹→3¹) | (5¹→3¹) |
| Endogenous | β- | TCCCTGGAGAAGAGC | GTAGTTTCGTGGATG |
| Control | ACTIN | TACG | CCACA |
| | | (SEQ ID NO: 9) | (SEQ ID NO: 10) |
| Neural | NESTIN | GAAACAGCCATAGAG | TGGTTTTCCAGAGTC |
| Crest | | GGCAA | TTCAGTGA |
| | | (SEQ ID NO: 21) | (SEQ ID NO: 22) |
| | PAX3 | TGTTTCCCTTTCCTG | TTATATCGCCTTGGG |
| | | TGTGG | CATTG |
| | | (SEQ ID NO: 13) | (SEQ ID NO: 14) |
| Odontoblast | RUNX2 | CATGGCGGGTAACGA | GTGAAGACGGTTATG |
| | | TGAAA | GTCAAGG |
| | | (SEQ ID NO: 23) | (SEQ ID NO: 24) |
| | DSPP | TGACAGCAATGATGA | CACTGGTTGAGTGGT |
| | | GAGTG | TACTG |
| | | (SEQ ID NO: 17) | (SEQ ID NO: 18) |
| | DMP1 | GACAGACAAGAAGGA | GCTCTCACTGGTGGT |
| | | GGAAAC | ATCT |
| | | (SEQ ID NO: 25) | (SEQ ID NO: 26) |

TABLE 4
Sci-RNA-Seq Based Signaling Pathways Predicted to Guide
Human Dental Papilla to Preodontoblast Transition.

| | Pathway | Percentage of Signaling |
|---|---|---|
| Pathway | Activity | Pathway Contribution to |
| FGF | 0.553875393409193 | 22.5% |
| BMP | 0.550038163958196 | 22.3% |
| NOTCH | 0.196219363745902 | 8.0% |
| ROBO | 0.183949373820091 | 7.5% |
| TGFb | 0.140973592959099 | 5.7% |
| HH | 0.139080487461313 | 5.6% |
| ACTIVIN | 0.138046901967685 | 5.6% |
| BMP10 | 0.0945780656332932 | 3.8% |
| WNT | 0.0918974676295192 | 3.7% |
| GDF | 0.0862911705726348 | 3.5% |
| ncWNT | 0.0734167231137553 | 3.0% |
| HGF | 0.0504931236916422 | 2.0% |
| EGF | 0.0304440629052946 | 1.2% |
| NODAL | 0.0259444591030694 | 1.1% |
| IGF | 0.0244637761693692 | 1.0% |
| NRG | 0.0243552503242357 | 1.0% |
| VEGF | 0.0158145446872169 | 0.6% |
| NT | 0.013599439781608 | 0.6% |
| GDNF | 0.0110040049089926 | 0.4% |
| PDGF | 0.0105747846085393 | 0.4% |
| NGF | 0.00679971989080399 | 0.3% |
| EDA | 0.00208039892802572 | 0.1% |

TABLE 5
Sci-RNA-Seq Based Dental Papilla to Signaling Ligands
Predicted to Guide Human Pre-Odontoblast Transition.

| | | | Percentage |
|---|---|---|---|
| | | Ligand | Contribution to |
| | | Activity | Pathway |
| Ligand | Pathway | Rank | Activity |
| GDF5 | BMP | 0.107936854104948 | 4.4% |
| FGF2 | FGF | 0.0951687697643382 | 3.9% |
| BMP10 | BMP10 | 0.0945780656332932 | 3.8% |
| SLIT2 | ROBO | 0.0919746869100457 | 3.7% |
| INHBA | ACTIVIN | 0.0861579837615462 | 3.5% |
| TGFB3 | TGFb | 0.0824392399327761 | 3.3% |
| WNT5A | ncWNT | 0.0734167231137553 | 3.0% |
| BMP15 | BMP | 0.0706731787268401 | 2.9% |
| BMP2 | BMP | 0.0706731787268401 | 2.9% |
| BMP4 | BMP | 0.0706731787268401 | 2.9% |
| BMP6 | BMP | 0.0706731787268401 | 2.9% |
| BMP7 | BMP | 0.0706731787268401 | 2.9% |
| SLIT1 | ROBO | 0.0637033830817625 | 2.6% |
| GDF9 | GDF | 0.0603467114695654 | 2.4% |
| TGFB2 | TGFb | 0.0585343530263231 | 2.0% |
| HGF | HGF | 0.0504931236916422 | 2.0% |
| GDF6 | BMP | 0.0481681205331745 | 1.9% |
| DHH | HH | 0.0463601624871043 | 1.9% |
| IHH | HH | 0.0463601624871043 | 1.9% |
| SHH | HH | 0.0463601624871043 | 1.8% |
| FGF1 | FGF | 0.044675646072696 | 1.7% |
| FGF4 | FGF | 0.0419522374212554 | 1.7% |
| FGF7 | FGF | 0.0419522374212554 | 1.6% |
| GDF7 | BMP | 0.0405672956858721 | 1.6% |
| DLK1 | NOTCH | 0.0392438727491804 | 1.6% |
| DLL1 | NOTCH | 0.0392438727491804 | 1.6% |
| DLL4 | NOTCH | 0.0392438727491804 | 1.6% |
| JAG1 | NOTCH | 0.0392438727491804 | 1.6% |
| JAG2 | NOTCH | 0.0392438727491804 | 1.5% |
| FGF10 | FGF | 0.0366807225255164 | 1.5% |
| FGF18 | FGF | 0.0366807225255164 | 1.5% |
| FGF19 | FGF | 0.0366807225255164 | 1.5% |
| FGF23 | FGF | 0.0366807225255164 | 1.5% |
| FGF3 | FGF | 0.0366807225255164 | 1.5% |
| FGF5 | FGF | 0.0366807225255164 | 1.5% |
| FGF6 | FGF | 0.0366807225255164 | 1.5% |
| FGF8 | FGF | 0.0366807225255164 | 1.5% |
| FGF9 | FGF | 0.0366807225255164 | 1.4% |
| WNT3A | WNT | 0.0345141279478644 | 1.1% |
| SLIT3 | ROBO | 0.0282713038282832 | 1.1% |
| GDF11 | GDF | 0.0259444591030694 | 1.1% |
| INHBB | ACTIVIN | 0.0259444591030694 | 1.1% |
| INHBC | ACTIVIN | 0.0259444591030694 | 1.1% |
| NODAL | NODAL | 0.0259444591030694 | 1.0% |
| IGF2 | IGF | 0.0244637761693692 | 0.9% |
| WNT1 | WNT | 0.0212302919460077 | 0.9% |
| WNT7B | WNT | 0.0212302919460077 | 0.6% |
| WNT4 | WNT | 0.0149227557896393 | 0.4% |
| PDGFB | PDGF | 0.0105747846085393 | 0.3% |
| NGF | NGF | 0.00679971989080399 | 0.3% |
| NTF3 | NT | 0.00679971989080399 | 0.3% |
| NTF4 | NT | 0.00679971989080399 | 0.2% |
| BTC | EGF | 0.00608881258105893 | 0.2% |
| EGF | EGF | 0.00608881258105893 | 0.2% |
| EREG | EGF | 0.00608881258105893 | 0.2% |
| HBEGF | EGF | 0.00608881258105893 | 0.2% |
| NRG1 | NRG | 0.00608881258105893 | 0.2% |
| NRG2 | NRG | 0.00608881258105893 | 0.2% |
| NRG3 | NRG | 0.00608881258105893 | 0.2% |
| NRG4 | NRG | 0.00608881258105893 | 0.2% |
| TGFA | EGF | 0.00608881258105893 | 0.2% |
| GDNF | GDNF | 0.00550200245449629 | 0.2% |
| NRTN | GDNF | 0.00550200245449629 | 0.2% |
| PGF | VEGF | 0.00527151489573897 | 0.2% |
| VEGFA | VEGF | 0.00527151489573897 | 0.2% |
| VEGFB | VEGF | 0.00527151489573897 | 0.2% |
| EDA | EDA | 0.00208039892802572 | 0.1% |

TABLE 6
Sci-RNA-Seq Based Signaling Pathways Predicted to Guide
Human Preodontoblast to Odontoblast Transition

| | | Percentage of Signaling |
|---|---|---|
| | Pathway | Pathway Contribution to |
| Pathway | Activity | Overall Signaling Activity |
| HH | 6.58091396461215 | 22.8% |
| NOTCH | 5.95770562015242 | 20.6% |
| BMP | 3.60889869037237 | 12.5% |
| ROBO | 2.61501748823247 | 9.1% |
| GDF | 1.97619555193484 | 6.8% |
| WNT | 1.89405110769015 | 6.6% |
| ncWNT | 1.34370262741258 | 4.7% |
| ACTIVIN | 1.21532494524332 | 4.2% |
| FGF | 1.05167605818864 | 3.6% |
| NT | 0.600320685338475 | 2.1% |
| BMP10 | 0.545134512038139 | 1.9% |
| HGF | 0.430411617253077 | 1.5% |
| NODAL | 0.405108315081107 | 1.4% |
| VEGF | 0.309959146896494 | 1.1% |
| NGF | 0.171735189235851 | 0.6% |
| TGFb | 0.140026196957032 | 0.5% |
| EDA | 0.00857715901768102 | 0.0% |

TABLE 7
Sci-RNA-Seq Based Signaling Ligands Predicted to Guide
Human Preodontoblast to Odontoblast Transition

| | | | Percentage |
|---|---|---|---|
| | | Ligand | Contribution to |
| Ligand | Pathway | Activity | Pathway Activity |
| DHH | HH | 2.19363798820405 | 7.6% |
| IHH | HH | 2.19363798820405 | 7.6% |
| SHH | HH | 2.19363798820405 | 7.6% |
| GDF9 | GDF | 1.57108723685373 | 5.4% |
| WNT5A | ncWNT | 1.34370262741258 | 4.7% |
| SLIT2 | ROBO | 1.30750874411624 | 4.5% |
| DLK1 | NOTCH | 1.19154112403048 | 4.1% |
| DLL1 | NOTCH | 1.19154112403048 | 4.1% |
| DLL4 | NOTCH | 1.19154112403048 | 4.1% |
| JAG1 | NOTCH | 1.19154112403048 | 4.1% |
| JAG2 | NOTCH | 1.19154112403048 | 4.1% |
| SLIT3 | ROBO | 0.929926855276543 | 3.2% |
| GDF5 | BMP | 0.908474659235602 | 3.1% |
| WNT3A | WNT | 0.840336283258083 | 2.9% |
| FGF2 | FGF | 0.795237112993616 | 2.8% |
| WNT4 | WNT | 0.734608917944184 | 2.5% |
| BMP10 | BMP10 | 0.545134512038139 | 1.9% |
| HGF | HGF | 0.430411617253077 | 1.5% |
| BMP15 | BMP | 0.405108315081107 | 1.4% |
| BMP2 | BMP | 0.405108315081107 | 1.4% |
| BMP4 | BMP | 0.405108315081107 | 1.4% |
| BMP5 | BMP | 0.405108315081107 | 1.4% |
| BMP6 | BMP | 0.405108315081107 | 1.4% |
| BMP7 | BMP | 0.405108315081107 | 1.4% |
| GDF11 | GDF | 0.405108315081107 | 1.4% |
| INHBA | ACTIVIN | 0.405108315081107 | 1.4% |
| INHBB | ACTIVIN | 0.405108315081107 | 1.4% |
| INHBC | ACTIVIN | 0.405108315081107 | 1.4% |
| NODAL | NODAL | 0.405108315081107 | 1.4% |
| SLIT1 | ROBO | 0.377581888839694 | 1.3% |
| GDF7 | BMP | 0.269774140650125 | 0.9% |
| BDNF | NT | 0.200106895112825 | 0.7% |
| NTF3 | NT | 0.200106895112825 | 0.7% |
| NTF4 | NT | 0.200106895112825 | 0.7% |
| NGF | NGF | 0.171735189235851 | 0.6% |
| WNT1 | WNT | 0.156057160151849 | 0.5% |
| FGF1 | FGF | 0.153119229562863 | 0.5% |
| TGFB3 | TGFb | 0.140026196957032 | 0.5% |
| WNT3 | WNT | 0.105727365313899 | 0.4% |
| FGF7 | FGF | 0.103319715632165 | 0.4% |
| PGF | VEGF | 0.103319715632165 | 0.4% |
| VEGFA | VEGF | 0.103319715632165 | 0.4% |
| VEGFB | VEGF | 0.103319715632165 | 0.4% |
| WNT2 | WNT | 0.028660690511067 | 0.1% |
| WNT7A | WNT | 0.028660690511067 | 0.1% |
| EDA | EDA | 0.00857715901768102 | 0.0% |

For all tables below, the dark line indicates samples from the incisor (above the dark line) and from the molar (below the dark line)

### Example 3

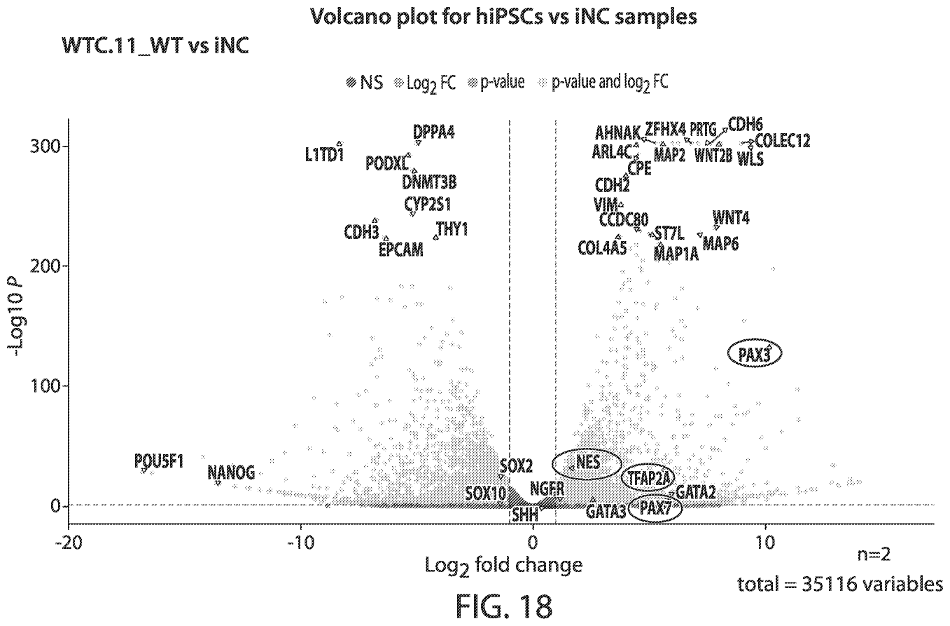

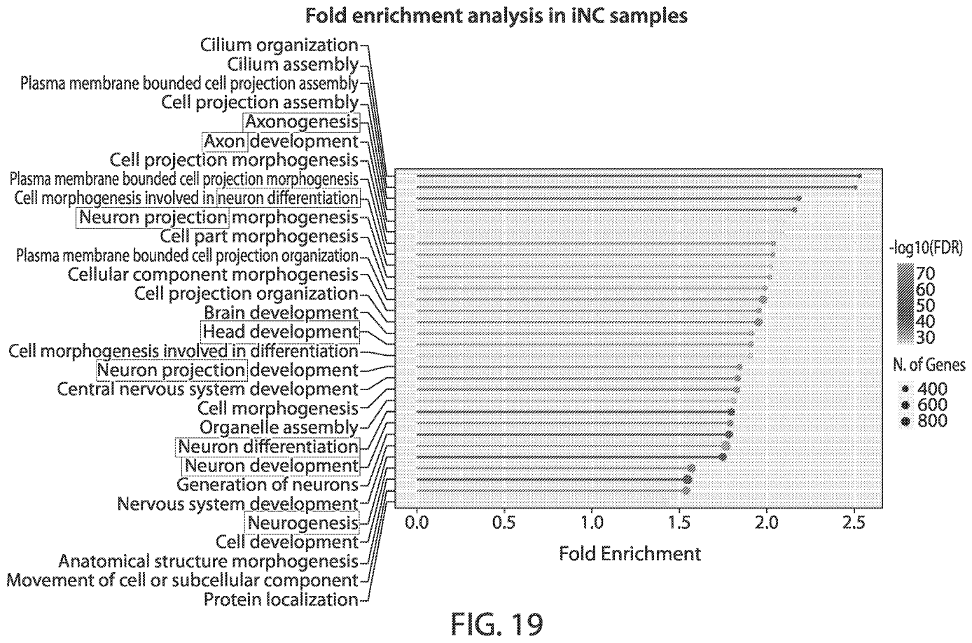

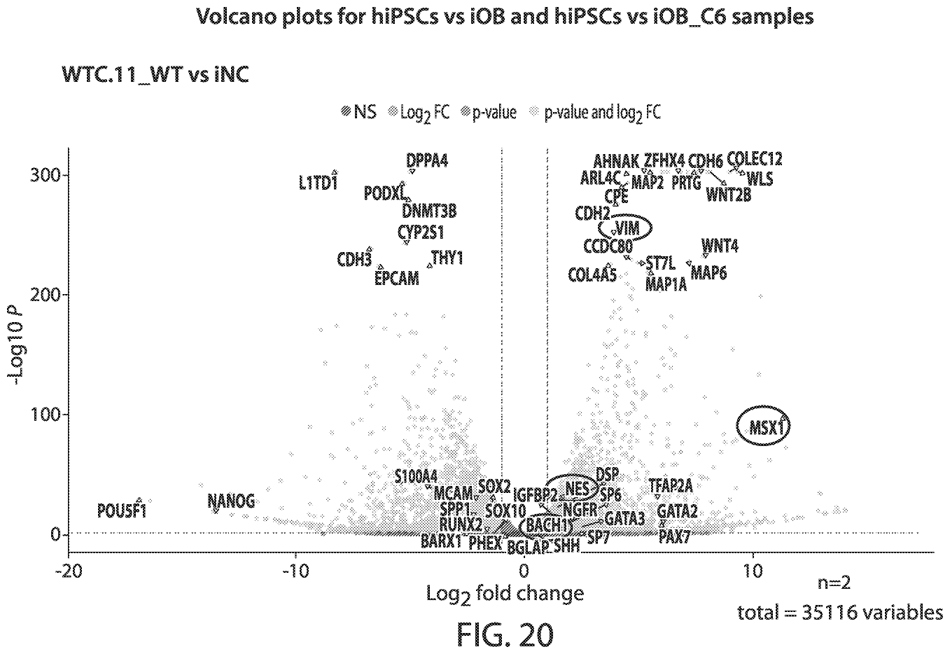

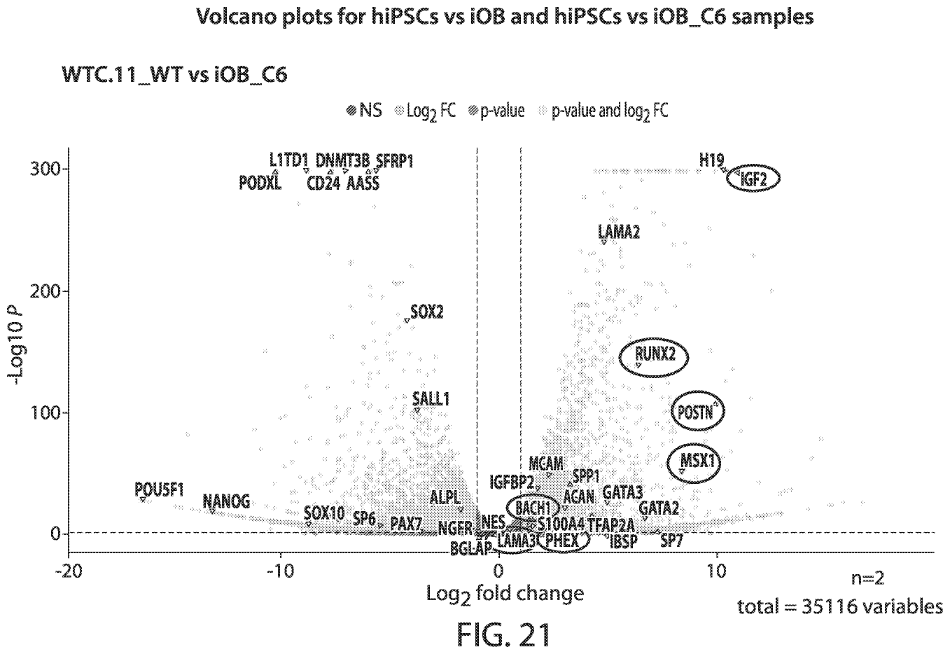

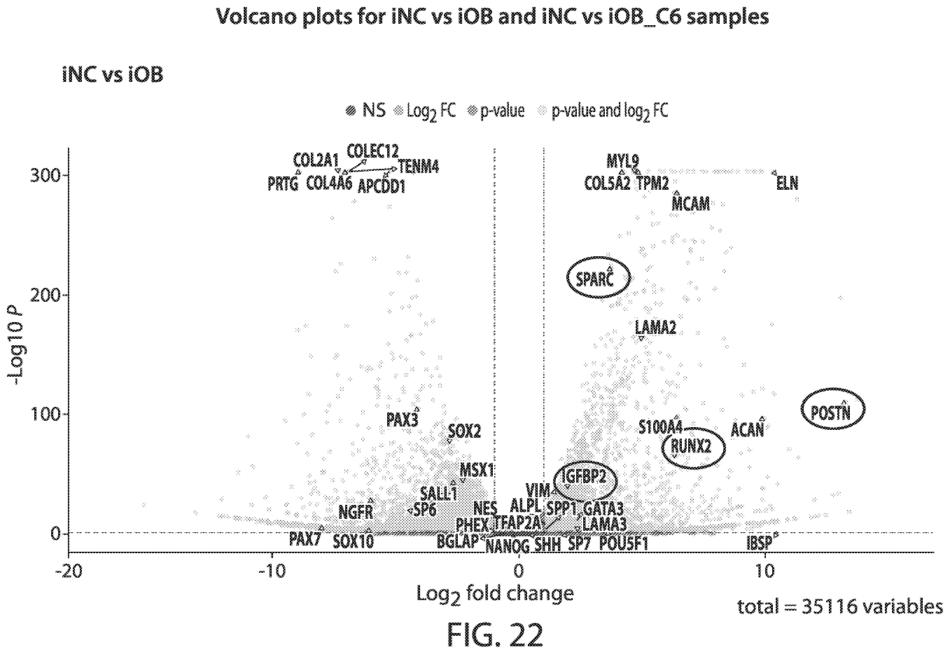

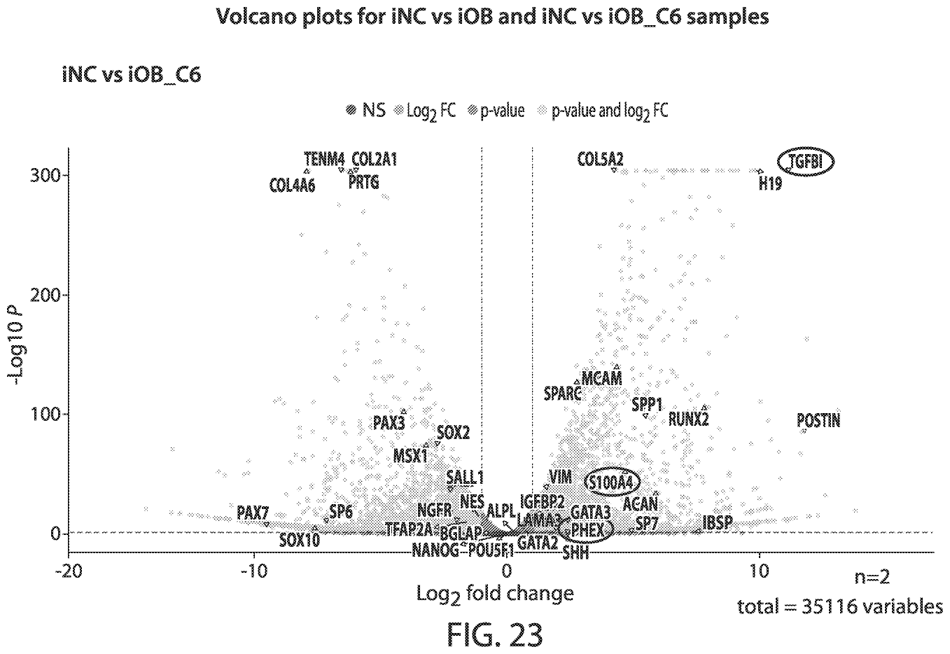

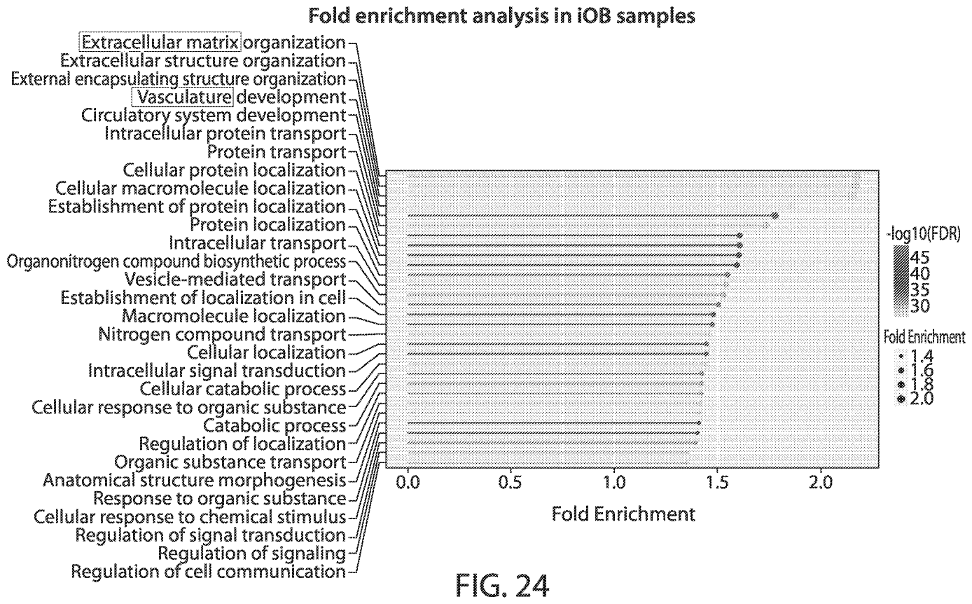

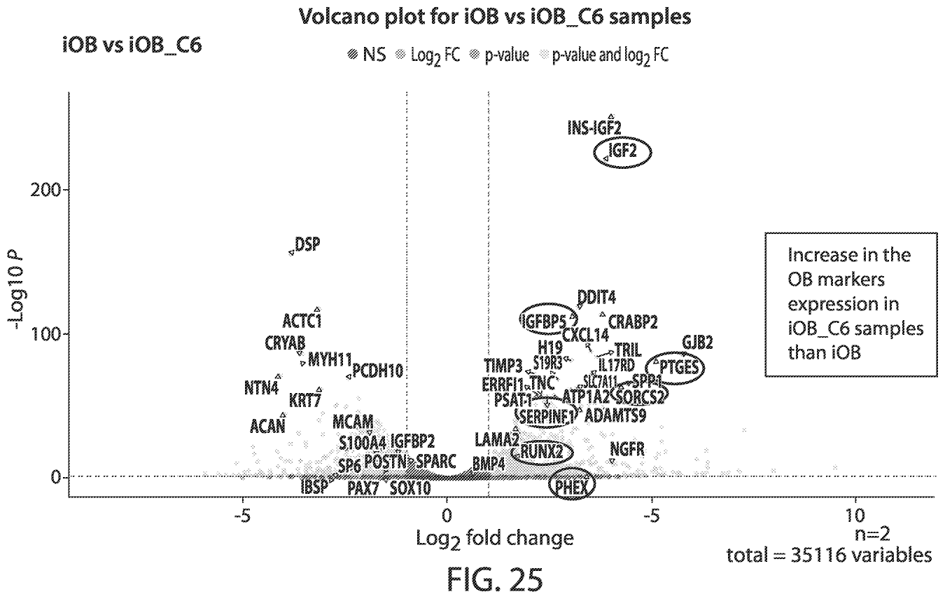

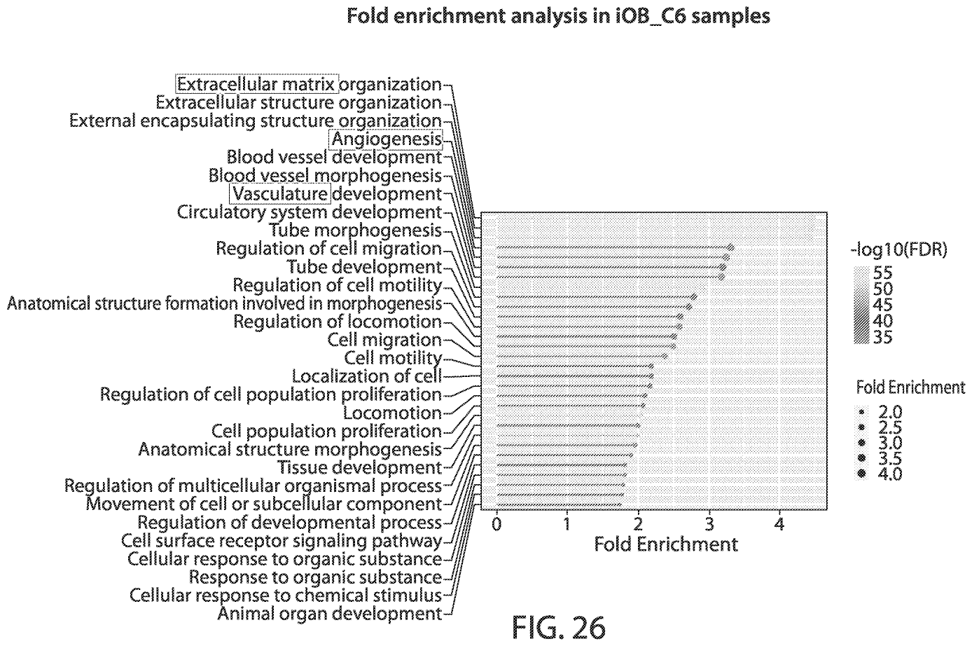

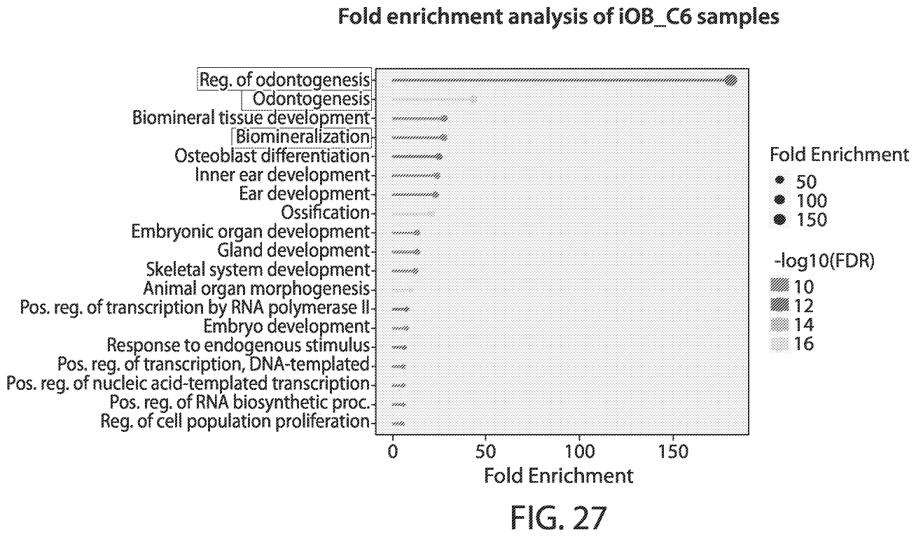

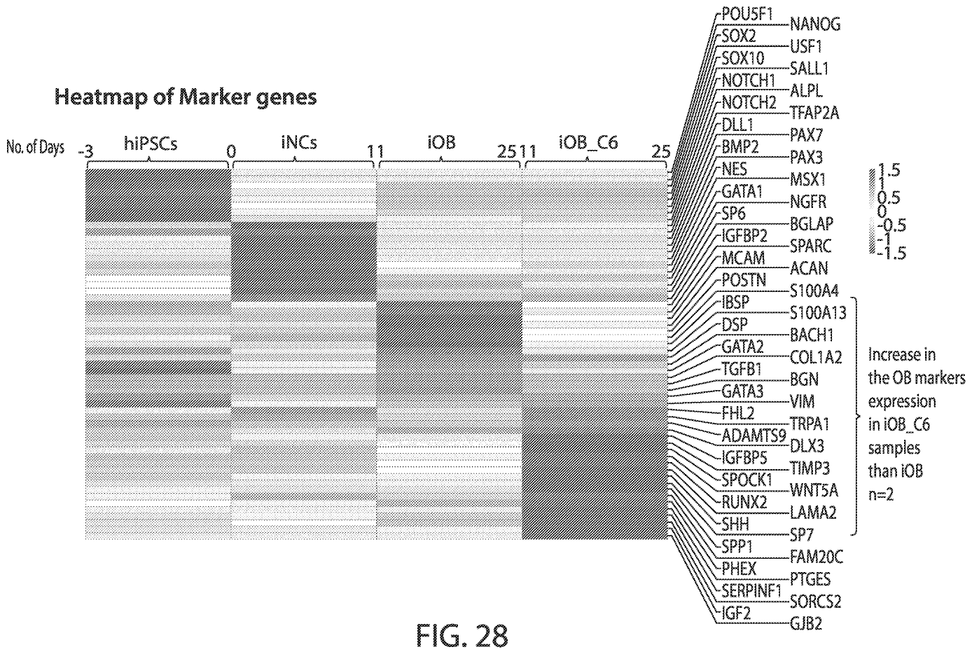

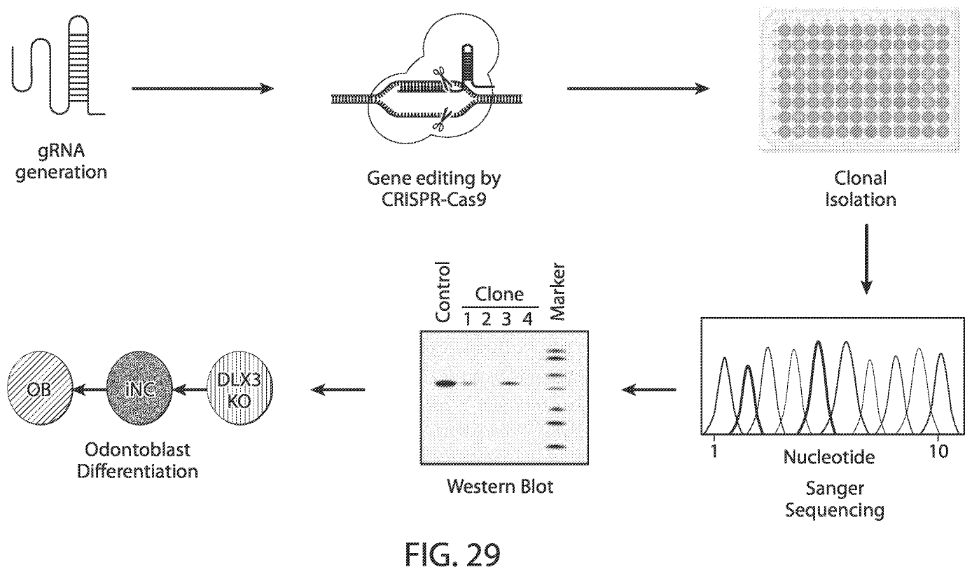

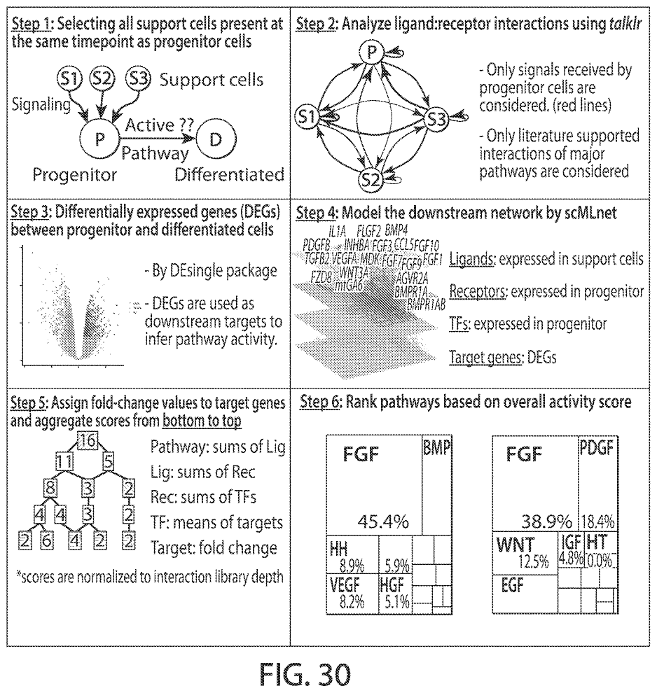

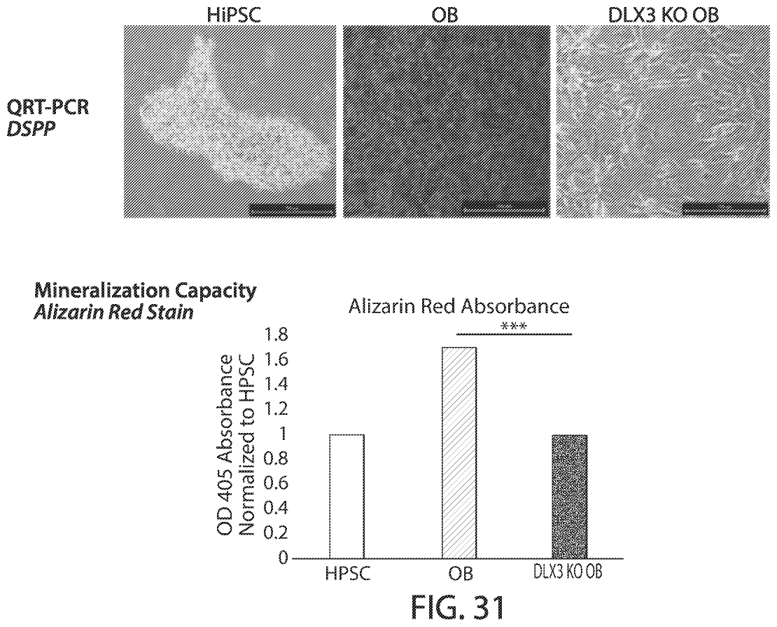

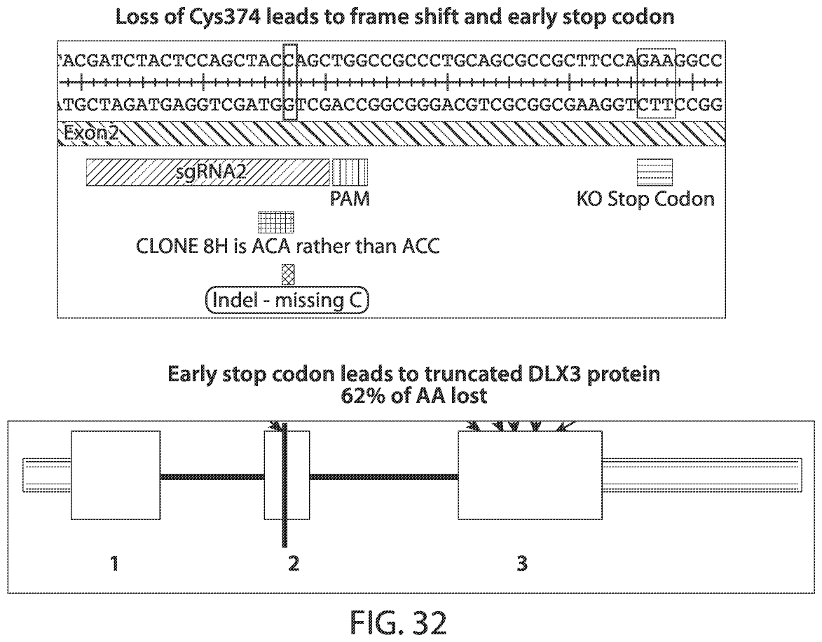

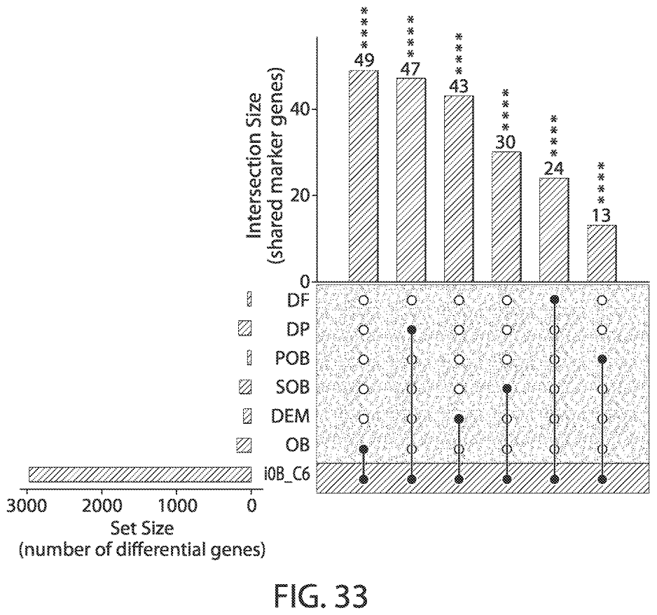

List of Overlapping genes found in each clusters (based on data from FIGS. 18-28, 33):

### OB:

SLC38A11, PHEX, COL1A2, COL1A1, ADAMTSI2, SPARC, PLOD2, CREB3L1, AFAP1, BGN, DOK6, NEAT1, NAV3, SEMA5A, SLCI3A5, DENND2A, ILIR1, ISM1, SDK2, TGFB2, CSDC2, RELN, MALATI, MIR3IHG, FRMD6, TMEM117, LOX, FAM20A, ELL2, PCDH7, PCDH9, APCDDI, P4HA1, CDKNIC, CSGALNACTI, SAMD4A, FKBP10, NKAIN2, SMAD6, FNDC3B, MEF2C, COL5A1, LRP1, COLEC12, TANC2, COL5A2, SGCD, SEC31A, PACS1

### DP:

SOX5, HMGA2, PRSS23, FGF14, DMD, PDE4D, CDH11, LIMCH1, PAM, FRMD4A, RAPIGDSI, HAPLNI, GRIK2, AFF3, LRP1B, CNTN5, ROR2, DPYD, DLC1, GPC6, TENM2, ME3, FBXL7, MNI, ADGRB3, LHFPL6, ARHGAP24, KAZN, EPHA7, INPP4B, HMCN1, KIAA1217, SLIT2, EDIL3, SSBP2, LEF1, PDE3A, ZFPM2, ZFHX4, TSHZ2, ZBTB20, FREM1, MIR99AHG, TRIO, CALD1, BMPR2, EXT1

### DEM:

ADGRL3, SMOC2, GREM2, RUNX2, EPHA3, KIF26B, SLIT3, EYA4, PRRX1, LAMA4, NCAM2, NCAM1, NEGRI, KANK4, PDE7B, EGFR, ZEB2, BICC1, NELL1, PBX3, RARB, SUGCT, BNC2, PRICKLE2, MAML2, TRPS1, SULF1, PALLD, MIR100HG, LSAMP, CHSY3, DNAJCI, ANTXRI, SPATS2L, SETBPI, NAALADL2, FLRT2, ATP2B4, TNS3, ZNF521, LPP, ATXN1, TTC3

### SOB:

KRT17, VIM, ACTG1, MYL6, TUBAIA, TMSB10, MARCKS, TMSB4X, JUN, S100A11, IGFBP4, LGALSI, ITM2B, FLNA, LUM, PCOLCE, CD63, DLK1, MFAP4, COL6A1, DLX3, CD248, MYH9, EMILINI, FSTL1, MSX1, COL4A2, P4HB, CPE, PLD3

### DF:

COL3A1, IGFBP5, CXCL14, COL6A2, SOSTDCI, DCN, MMP14, FBLN1, INHBA, COLIIA1, COLI6A1, SLC8A1, FBN1, SCUBE3, TIMP3, COL6A3, FN1, COL4A1, MXRA8, NFIA, POSTN, LAMB1, LMNA, TPM4

### POB:

FBN2, CDH13, GLIS1, PDGFD, UNC5C, GREBIL, NFIC, PDCD4, FOSB, GRK5, CPED1, TANC1, TNRC18

1. A method of preparing an odontoblast, the method comprising, in order
a) contacting at day zero, in culture, a pluripotent stem cell with a TGF-β/SMAD inhibitor;
b) adding a WNT activator to the culture in (a) at day 2;
c) enriching the culture of step (b) for a population of induced neural crest stem cells;
d) contacting the population of induced neural crest stem cells enriched in (c) with an odontoblast differentiation medium comprising a BMP pathway agonist, an FGF pathway agonist, and a Hedgehog pathway agonist, and incubating to generate a population of odontoblast cells expressing dentin sialophosphoprotein (DSPP).

2. The method of claim 1, wherein the pluripotent stem cell is at least one of:
an induced pluripotent stem cell (iPSC),
a human pluripotent stem cell,
are grown to confluence prior to step (a),
are cultured to confluence in mTeSR1 stem cell medium,
comprises a mutation inactivating expression or activity of DLX3, and
is seeded on tissue culture plates coated with an extracellular matrix composition.

3. The method of claim 1, wherein steps (a) through (c) are performed in a basal neural maintenance medium (BNMM) to which the SMAD inhibitor and WNT activator are successively added or added and removed.

4. The method of claim 3, wherein the BNMM comprises: Dulbecco's Modified Eagle Medium F12+glutamine: neurobasal medium (1:1), wherein the neurobasal medium comprises a N2 supplement, B27, Glutamax, ITS-A, b-mercaptoethanol, and non-essential amino acids (NEAA).

5. The method of claim 3, wherein at least one of:
the SMAD inhibitor comprises SB431542 and LDN193189,
the WNT activator is a GSK-3 inhibitor, and
the WNT activator is CHIR99021.

6. The method of claim 5, wherein the SB431542 is removed at day 4 of culture, and LDN193189 is removed at day 3 of culture, and/or
wherein the WNT activator is added from day 2 to day 11 of the method.

7.-10. (canceled)

11. The method of claim 1, wherein enriching step (c) comprises selection of cells expressing p75^{NTR},
wherein enriching step (c) is performed when a majority of the differentiating cells expresses p75^{NTR}, and/or
wherein the enriching step (c) is performed at day 11.

12.-14. (canceled)

15. The method of claim 1, wherein at least one of:
the BMP pathway agonist comprises BMP4,
the FGF pathway agonist is selected from bFGF, FGF8b and an FGF receptor minibinder, and
the Hedgehog pathway agonist comprises Smoothened agonist (SAG).

16. (canceled)

17. The method of claim 15, wherein the FGF receptor mini binder is selected from mb7 or mb6 receptor mini binder.

18.-24. (canceled)

25. The method of claim 2, wherein the iPSCs are cultured to confluence in mTeSR1 stem cell medium.

26. The method of claim 2, wherein confluent pluripotent stem cells are switched to a basal neural crest maintenance medium at day zero of differentiation.

27. The method of claim 1, wherein the TGF-β/SMAD inhibitors are added for at least 3 days.

28. The method of claim 1, wherein the induced neural crest cell expresses p75, AP-2α, NESTIN, and/or PAX3.

29. (canceled)

30. (canceled)

31. The method of claim 1, wherein the BMP4 pathway agonist is at least one of:
added from Day 11 to Day 17 of differentiation,
added from Day 17 to Day 26 of differentiation,
at a concentration from 25 ng/mL to 100 ng/mL, and
at a concentration of 50 ng/mL.

32.-34. (canceled)

35. The method of claim 15, wherein SAG is added at a concentration of 200 nM to 1 μM, or at a concentration of 400 nm.

36. (canceled)

37. A human odontoblast produced by the method of claim 1, wherein the FGF agonist is an FGF receptor mini binder, and the odontoblast exhibits at least 10% greater mineralization than an odontoblast differentiated without the FGF receptor mini binder.

38. A composition comprising an in vitro-differentiated human odontoblast and a biodegradable scaffold.

39.-45. (canceled)

46. A co-culture comprising an odontoblast produced by the method of claim 1 and an in vitro-differentiated ameloblast.

47.-60. (canceled)

61. A method of treating a dental disease or disorder, the method comprising administering a composition comprising an in vitro-differentiated odontoblast to a subject in need thereof.

62. The method of claim 61, wherein a dental disease or disorder is selected from Tricho-Dento-Osseous (TDO) syndrome, amelogenesis imperfecta, periodontal disease, periodontitis, canes, pericoronitis, pulpitis, enamel hypoplasia, defects of dentition, and tartar.

63.-74. (canceled)
